(19) United States
(12) Patent Application Publication
(10) Pub. No.: US 2024/0016838 A1
(43) Pub. Date: Jan. 18, 2024

(54) ENGINEERED NK CELLS

(71) Applicant: City of Hope, Duarte, CA (US)

(72) Inventors: Srividya Swaminathan, Duarte, CA (US); Anil Kumar, Duarte, CA (US); Sung June Lee, Duarte, CA (US); Adeleh Taghi Khani, Duarte, CA (US); Chun-Wei Chen, Duarte, CA (US)

(21) Appl. No.: 18/033,777

(22) PCT Filed: Oct. 26, 2021
(86) PCT No.: PCT/US21/56714

§ 371 Date: Apr. 25, 2023

Related U.S. Application Data
(60) Provisional application No. 63/105,833, filed on Oct. 26, 2020.
(60) Provisional application No. 63/115,482, filed on Nov. 18, 2020.
(60) Provisional application No. 63/186,039, filed on May 7, 2021.

(51) Int. Cl. A61K 35/17; C12N 5/0783; C12N 15/11; C12N 9/22; C12N 15/86; C12N 15/90; A61P 35/02

(52) U.S. Cl. CPC A61K 35/17; C12N 5/0646; C12N 15/11; C12N 9/22; C12N 15/86; C12N 2830/005; A61P 35/02; C12N 2310/20; C12N 2800/80; C12N 2740/15043; C12N 15/907

(57) ABSTRACT

Provided herein, inter alia, are compositions including engineered NK cells and methods for preparing the same. The engineered NK cells provided herein include integrated nucleic acid sequences encoding Cas9 proteins (e.g. dCas9). The engineered NK cells are contemplated to be effective for treating and/or preventing cancer, particularly leukemia.

### CROSS-REFERENCE TO RELATED APPLICATIONS

This application claims priority to U.S. Provisional Application No. 63/105,833, filed Oct. 26, 2020, U.S. Provisional Application No. 63/115,482, filed Nov. 18, 2020, and U.S. Provisional Application No. 63/186,039, filed May 7, 2021, which are hereby incorporated by reference in their entirety and for all purposes.

### STATEMENT AS TO RIGHTS TO INVENTIONS MADE UNDER FEDERALLY SPONSORED RESEARCH AND DEVELOPMENT

This invention was made with government support under grant no. P50 CA107399-12 awarded by the National Institutes of Health. The government has certain rights in the invention.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048440-783001WO_SequenceListing_ST25.TXT, created on Oct. 26, 2021, 32,768 bytes, machine format IBM-PC, MS Windows operating system, is hereby incorporated by reference.

### BACKGROUND

Natural Killer (NK) cells are a type of cytotoxic lymphocyte critical to the innate immune system. The role of NK cells is analogous to that of cytotoxic T cells in the vertebrate adaptive immune response. NK cells provide rapid responses to virus-infected cells, acting at around 3 days after infection. NK cells further respond to tumor formation. The role of NK cells in both the innate and adaptive immune responses is becoming increasingly important in research using NK cell activity as a potential cancer therapy. Based on our knowledge, no other groups thus far have been successful in constitutively expressing CAS9 and dCAS9 in human NK cell line for CRISPR-based therapeutic applications.

Provided herein, inter alia, are solutions to these and other problems in the art.

### BRIEF SUMMARY

In an aspect is provided a Natural Killer (NK) cell including an integrated nucleic acid sequence encoding a catalytically inactive Cas9 (dCas9) protein.

In another aspect is provided a substantially pure population of Natural Killer (NK) cells, wherein each of the NK cells includes an integrated nucleic acid sequence encoding a Cas9 protein.

In another aspect is provided a substantially pure population of Natural Killer (NK) cells, wherein each of the NK cells includes an integrated nucleic acid sequence encoding a catalytically inactive Cas9 (dCas9) protein.

In another aspect is provided a method of preparing a plurality of Natural Killer (NK) cells each including an integrated nucleic acid sequence encoding a Cas9 protein, the method including: (a) contacting a plurality of NK cells with a vector comprising a nucleic acid encoding a Cas9 protein thereby producing a transduced NK Cell wherein the nucleic acid encoding the Cas9 protein is integrated into the genome of the NK Cell; (b) separating the transduced NK cell from the plurality of NK cells; and (c) expanding the transduced NK cell thereby producing a plurality of natural Killer (NK) cells each including an integrated nucleic acid sequence encoding a Cas9 protein.

In another aspect is provided a plurality of Natural Killer (NK) cells each including an integrated nucleic acid sequence encoding a Cas9 protein provided herein, including embodiments thereof.

In another aspect is provided a method of preparing a plurality of Natural Killer (NK) cells each including an integrated nucleic acid sequence encoding a catalytically inactive Cas9 (dCas9) protein, the method including: (a) contacting a plurality of NK cells with a vector comprising a nucleic acid encoding a dCas9 protein thereby producing a transduced NK Cell wherein the nucleic acid encoding the dCas9 protein is integrated into the genome of the NK Cell; (b) separating the transduced NK cell from the plurality of NK cells; and (c) expanding the transduced NK cell thereby producing a plurality of natural Killer (NK) cells each including an integrated nucleic acid sequence encoding a dCas9 protein.

In another aspect is provided a plurality of Natural Killer (NK) cells each comprising an integrated nucleic acid sequence encoding a dCas9 protein provided herein, including embodiments thereof.

In another aspect is provided a method of increasing expression of a target gene within a subject, the method including administering to the subject a plurality of Natural Killer (NK) cells, wherein each of the plurality NK cell includes an integrated nucleic acid sequence encoding a catalytically inactive Cas9 (dCas9).

In another aspect is provided a method of decreasing the expression of a target gene within a subject, the method including administering to the subject a plurality of NK cells, wherein each of the plurality NK cells includes an integrated nucleic acid sequence encoding a Cas9.

In another aspect is provided method of treating or preventing a disease in a subject in need thereof, the method including administering to the subject a therapeutically effective amount of an NK cell capable of expressing an elevated level of a type I interferon or an elevated level of IL-15 relative to a standard control NK cell.

In an aspect is provided a method of preventing recurrence of leukemia in a subject, the method including administering to the subject an effective amount of an natural killer (NK) cell capable of expressing an elevated level of a type I interferon relative to a standard control NK cell or an elevated level of IL-15 relative to the standard control NK cell.

In an aspect a method of treating or preventing an infectious disease in a subject having leukemia is provided, the method including administering an effective amount of a natural killer (NK) cell capable of expressing an elevated level of a type I interferon relative to a standard control NK cell or an elevated level of IL-15 relative to the standard control NK cell.

### BRIEF DESCRIPTION OF THE DRAWINGS

FIGS. 1A-1C illustrate workflow for generating and validating human NK cells constitutively expressing CAS9 and dCAS9 for therapeutic applications. FIG. 1A, is a workflow for generation and validation of stable Cas9 expressing NK-92 cells. FIG. 1B, is a workflow for generation and validation of stable dCas9-VP64 expressing NK-92 cells. FIG. 1C, is a workflow for generation of stable dCas9-KRAB expressing NK-92 cells.

FIGS. 2A-2B illustrate the generation of stable Cas9-EGFP+ NK-92 cells by lentiviral transduction and constitutive overexpression of Cas9 in NK-92 cells. FIG. 2A presents flow cytometry dot plots showing the percentages of transduced EGFP+ cells on days 2, 11 and 15 post Lenti-CAS9-EGFP transduction. FIG. 2B presents dot plots showing the pre-sort percentages on the day of sort (day 0) and post-sort purity on days 0 and 10.

FIG. 3 illustrates the isolation of monoclonal Cas9-EGFP+ NK-92 cells using limiting dilution technique. FIG. 3 presents flow cytometry dot plots depicting the percentages of polyclonal and monoclonal Cas9-EGFP+ NK-92 cells (clone-1 to clone-9).

FIGS. 4A-4B illustrate the selection and analysis of monoclonal Cas9-EGFP+ NK-92 cell clone with best CRISPR editing efficiency. CAS9 expressing NK-92 single cells clones (5-9) were transduced with control sgRNA-RFP or RFP sgRNA-RFP. FIG. 4A presents flow cytometry dot plots showing the percentages of RFP+ cells on day 2 post transduction. FIG. 4B presents flow cytometry dot plots showing the percentages of RFP+ cells in puromycin selected monoclonal Cas9−EGFP+ NK-92 cells on day 10.

FIGS. 5A-5B illustrate validation of IL2Rγ knockout in Cas9-EGFP+ NK-92 cells using sgRNA-1 against IL2Rγ. Cas9-EGFP+ NK-92 cells (clone-6) were transduced with control sgRNA-RFP (IL2Rγ sgRNA-1-RFP−) on Cas9-EGFP+ NK-92 cells. FIG. 5A presents flow cytometry dot plots showing the percentages of RFP+ cells on different days post sgRNA transduction. FIG. 5B presents histogram overlays showing expression levels of IL-2Rγ chain on sgRNA transduced (RFP+) Cas9-EGFP+ NK-92 cells. The values for the control sgRNA-RFP experiment are shown in the top right corner of each histogram overlay (e.g., “4763” at day 1) and values for the IL2Rγ sgRNA-1-RFP experiment are shown below the values for the control experiment (e.g., “1484” at day 1).

FIGS. 6A-6B illustrate the validation of IL2Rγ knockout in Cas9-EGFP+ NK-92 cells using sgRNA-2 against IL2Rγ. IL-2Rγ chain knock-out in Cas9-EGFP+ NK-92 cells. Cas9-EGFP+ NK-92 cells (clone-6) were transduced with control sgRNA-RFP (IL2Rγ sgRNA-2-RFP−) on Cas9-EGFP+ NK-92 cells. FIG. 6A presents flow cytometry dot plots showing the percentages of RFP+ cells on different days post sgRNA transduction. FIG. 6B presents histogram overlays showing expression levels of IL-2Rγ chain on sgRNA transduced (RFP+) Cas9-EGFP+ NK-92 cells. The values for the control sgRNA-RFP experiment are shown in the top right corner of each histogram overlay (e.g., “4763” at day 1) and values for the IL2Rγ sgRNA-1-RFP experiment are shown below the values for the control experiment (e.g., “2547” at day 1).

FIGS. 7A-7D illustrate the generation and validation of dCas9-VP64-GFP+ NK-92 cell line. NK-92 cells were transduced with concentrated LentidCas9-VP64-GFP virus. FIG. 7A presents flow cytometry dot plots showing the percentages of transduced cells on day 2 post transduction (7A top panel). Cells were expanded and sorted on day 11 using FACS method, dot plots (7A middle and bottom panels) showing the pre-sort percentages and post-sort purity of Lenti-Cas9-NK-92 cells. FIG. 7B presents flow cytometry analysis showing post sort purity on day 10. FIG. 7C presents flow cytometry analysis showing the percentages of control sgRNA and CRISPRa IL-15 sgRNA transduced dCAS9− VP64-GFP+ NK-92 cell day 2 post transduction (FIG. 7C, upper panel) and after puromycin selection (FIG. 7C, lower panel). FIG. 7D presents fold change in IL-15 expression levels between dCAS9-VP64-GFP+ NK-92 cells transduced with either negative control sgRNARFP- or CRISPRa IL-15 sgRNA-RFP.

FIGS. 8A-8B illustrate the generation of dCas9-KRAB-GFP+ NK-92 cell line. NK-92 cells were transduced with concentrated Lenti-dCAS9-KRAB-GFP virus. FIG. 8A presents flow cytometry dot plots showing the percentages of transduced cells on day 2, day 3 and day 8 post transduction. Cells were expanded for 22 days and were sorted using flow cytometry. FIG. 8B presents flow analysis showing the pre-sort and post-sort percentages of dCas9− KRAB-GFP+ NK-92 cells.

FIGS. 9A-9B illustrate the analysis of cytolytic potential of Cas9-, dCas9-VP64- and dCas9-KRAB-transduced NK-92 cells. It also shows constitutive expression of CAS9 or dCAS9 in NK cells which does not impair NK cytotoxicity. FIG. 9A presents flow cytometry showing percentages of 7-AAD+ CFSE-labeled target K562 erythroleukemia cells when co-cultured with unmodified or Cas9/dCas9-modified NK-92 polyclonal effector cells. FIG. 9B presents flow cytometry analysis showing the percentages of 7-AAD+ CFSE-labeled target K562 cells when co-cultured with monoclonal Cas9-EGFP NK-92 cells. Effector:target=10:1.

FIG. 10 illustrates the introduction of CAS9 and dCAS9-VP64 that do not impact NK cell proliferation. Histogram overlays showing changes in CFSE-violet fluorescence in untransduced, Cas9-transduced and dCas9-VP64 transduced NK-92 cells 4 days after CFSE labeling of the cells. Control=CFSE fluorescence in untransduced NK-92 cells immediately after labeling.

FIGS. 11A-11B illustrate the transcriptional activation of IL15 in NK cells by CRISPRa promotes NK cell-mediated lysis of sensitive targets. FIG. 11A presents flow cytometry showing percentages of 7-AAD⁺ CFSE-labeled target K562 erythroleukemia cells when co-cultured with effector dCAS9-VP64⁺ NK-92 cells expressing either control sgRNA or IL-15 sgRNA to transcriptionally activate IL15. FIG. 11B presents specific cytotoxicity of dCas9-VP64⁺ NK-92 cells expressing control sgRNA or IL-15 sgRNA at different effector to target cell ratios.

FIGS. 12A-12B illustrate the transcriptional activation of Type I IFNa2 in dCAS9-VP64+ NK-92 cells. FIG. 12A presents flow cytometry analysis showing the percentages of dCAS9-VP64-GFP+ NK-92 cells transduced with control sgRNA-RFP and IFNα2 sgRNA-RFP to transcriptionally activate IFNα2 on day 2 (upper panel) and day 9 (lower panel) post transduction and puromycin selection. FIG. 12B presents fold change in IFNα2 mRNA expression levels in dCAS9-VP64-GFP⁺ NK-92 cells transduced with either control sgRNA-RFP or three different IFNα2 sgRNA-RFP to transcriptionally activate IFNα2 using quantitative RT-PCR.

FIGS. 13A-13C illustrate the transcriptional activation of Type I IFNα1 and IFNβ1 in dCas9-VP64-GFP⁺ NK-92 cells. FIG. 13A presents flow cytometry analysis showing the percentages of dCAS9-VP64-GFP⁺ NK-92 cells transduced with IFNα1 sgRNA-RFP or IFNβ1 sgRNA-RFP to transcriptionally activate IFNα1 or IFNβ1, respectively, on day 2 (upper panel) and day 9 (lower panel) post transduction and puromycin selection. FIGS. 13B-13C present quantitative RT-PCR depicting fold change in IFNα1 and IFNβ1 mRNA expression in dCAS9-VP64-GFP⁺ NK-92 cells transduced with either control sgRNA-RFP or two different sgRNA-RFP each to transcriptionally activate IFNa2 and IFNb1.

FIG. 14 illustrates the introduction of Cas9, dCas9-VP64 and dCas9-KRAB into NK-92 cells does not block NK cell proliferation. Histogram overlays from three independent experiments showing CFSE-violet fluorescence at day 0 and decrease in CFSE-violet fluorescence at day 4 after cell proliferation. The MFI at day 0 and day 4 values are shown on each histogram.

FIG. 15 illustrates proliferation potential of Cas9, dCas9-VP64 and dCas9-KRAB transduced NK-92 cells. Histogram overlays from three independent experiments showing CFSE-violet fluorescence at day0 and decrease in CFSE-violet fluorescence at day4 after cell proliferation.

FIGS. 16A-16B illustrate validation of IL-2Rγ repression in dCas9-KRAB-GFP⁺ NK-92 cells using sgRNA-1 and -2 against IL2Rg. The figures present dCas9-KRAB-GFP⁺ NK-92 cells that were transduced with control sgRNA-RFP or two different sgRNA targeting IL-2Rγ chain (IL2Rg sgRNA-1-RFP, and IL2Rg sgRNA-2-RFP) on dCas9-KRAB-GFP⁺ NK-92 cells. The results demonstrate successful repression of IL-2Rγ chain expression in dCas9-KRAB-GFP⁺ NK-92 cells. FIG. 16A illustrates flow cytometry dot plots showing the percentages of RFP⁺ cells on different days post sgRNA transduction. FIG. 16B illustrates histogram overlays showing expression levels of IL-2Rγ chain on sgRNA transduced (RFP⁺) dCas9-KRAB-GFP⁺ NK-92 cells.

FIG. 17 illustrates plots showing the fold change in sgRNA-RFP⁺ dCas9-KRAB-GFP⁺ NK-92 cells with respect to day 1 post-transduction.

FIGS. 18A-18B illustrate transcriptional activation of IFNα2 in NK cells by CRISPRa does not affect their cytotoxicity. FIG. 18A presents flow cytometry showing percentages of 7-AAD⁺ CFSE-labeled target K562 erythrocytoleukemia cells when co-cultured with effector dCAS9-VP64⁺ NK-92 cells expressing either control sgRNA or IFNa2 sgRNA to transcriptionally activate IFNa2. FIG. 18B illustrates specific cytotoxicity of dCas9-VP64⁺ NK-92 cells expressing control sgRNA or IFNa2 sgRNA at different effector to target cell ratios.

FIGS. 19A-19E illustrate transcriptional activation of Type I interferon (IFN) and IL15 in dCas9-VP64-GFP⁺ NK-92 cells. FIG. 19A presents flow analysis showing the generation of dCas9-VP64-GFP⁺ NK92 cells followed by their FIG. 19B cytotoxicity analysis as compared to untransduced NK92 cells. FIG. 19C presents a cartoon showing cloning of type I interferons and IL15 sgRNA into single vector using STRING method as described by Breunig et al., Plos one, 2018. FIG. 19D presents flow cytometry analysis showing the percentages of dCAS9-VP64-GFP⁺ NK-92 cells transduced with control sgRNAs-RFP unit or type I interferon and IL15 sgRNAs-RFP unit, on day 2 (upper panel) and day 16 (lower panel) post transduction and puromycin selection. FIG. 19E presents validation of transcriptional activation of type I interferons (IFNα1, IFNα2 and IFNβ1) and IL15 in dCAS9-VP64-GFP⁺ NK-92 cells transduced with either control sgRNAs unit or type I interferon and IL15 sgRNAs unit.

FIG. 20 shows a schematic illustrating the generation of engineered NK cells provided herein and validation of the engineered cells by flow analysis or qPCR.

FIGS. 21A-21H illustrate data showing the generation and validation of the Cas9+NK-92 cell line. FIG. 21A is flow cytometry data showing detection of EGFP at day 2 and day 11 after transduction. FIG. 21B is flow cytometry data showing detection of EGFP at day 0 and day 10 after sorting of Cas9-EGFP+ cells. The data illustrate that the population of cells are substantially pure for Cas9+ cells. FIG. 21C. is flow data illustrating the proliferation of untransduced NK-92 cells and Cas9+ NK-92 cells. FIG. 21D is data showing detection of EGFP for single cell cloning of Cas9+ cells. FIG. 21E. is flow cytometry data illustrating the selection of clones from FIG. 21D with the best Cas9 nuclease activity by detecting reduction of RFP and presence of Cas9 by detection of EGFP. FIG. 21F is a bar graph illustration cytotoxicity of the clone selected in FIG. 21E on leukemia target cells. The ratio of NK effector cells to leukemia target cells was 12.5:1. FIG. 21G is data illustrating detection of EGFP and reduction of RFP for IL2R-gamma sgRNA and control sgRNA transduced Cas9-EGFP+ NK-92 cells. FIG. 21H is a graph showing fold change in sgRNA-RFP+ Cas9-GFP+ cells days after transduction.

FIGS. 22A-22F. illustrate data showing generation and validation of the dCas9-KRAB+ NK-92 cell line. FIG. 22A illustrates data showing detection of GPF for untransducted and dCAS9-KRAB-GFP transduced NK-92 cells from day 2 to day 22 after transduction. FIG. 21B shows data illustrating detection of GFP at day 0 and day 10 after sorting the dCas9-KRAB-GFP+ cells. The data shows that the population of cells are substantially pure for dCas9-KRAB-GFP+ cells FIG. 22C shows proliferation of untransduced and dCas9-KRAB+ NK-92 cells. FIG. 22D. illustrates detection of GPF for dCas9-KRAB-GPF single cell clones. FIG. 22E illustrates cytotoxicity of the untransduced and the sorted dCas9-KRAB+-NK-92 cells from FIG. 22D. The ratio of NK effector to leukemia target cells are 10:1. FIG. 22F illustrates validation of dCas9-DRAB-GFP+NK-92 cells by detecting reduction of RFP at day 13 compared to day 1.

FIGS. 23A-23G. show generation and validation of the dCas9-VP64+ NK-92 cell line. FIG. 23A illustrates detection of GFP in untransduced cells and dCas9-VP64-GFP transduced cells at day 2 and day 11 after transduction. FIG. 23B. illustrates detection of detection of GFP at day 0 and day 10 after sorting the cells. The results illustrate that the population of cells are substantially pure for dCas9-VP64-GFP cells. FIG. 23C. shows the proliferation of NK-92 cells and the dCas9-VP64+ NK-92 cells. FIG. 23D. illustrates single cell cloning of dCas9-VP64-GFP cells. FIG. 23E illustrates the cytotoxicity of the clone selected from FIG. 23D on leukemia cells. The ratio of NK effector cells to leukemia target cells is 10:1. FIG. 23F illustrates validation results of the dCas9-VP64-GFP+ NK-92 cells. The results show detection of RFP at day 2 (sgRNA transduction) and day 15 (puromycin selection). FIG. 23G shows fold change in IL-15 mRNA levels in cells transduced with control sgRNA and IL-15 sgRNA.

### DETAILED DESCRIPTION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

In this disclosure, “comprises,” “comprising,” “containing” and “having” and the like can have the meaning ascribed to them in U.S. patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “integrated nucleic acid sequence” as used herein refers to an exogenous nucleic acid sequence which has been inserted into the host chromosomal DNA. For example, a Natural Killer (NK) cell including an integrated nucleic acid sequence encoding a Cas9 protein (e.g. dCas9) refers to an NK cells wherein the nucleic acid sequence encoding the Cas9 protein is integrated into the chromosome of NK cell.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The term “lentiviral vector” or the like are used in accordance with their plain ordinary meaning. A lentivirus is typically a type of retrovirus that can infect both dividing and non-dividing cells because their pre-integration complex (virus “shell”) can get through the intact membrane of the nucleus of the target cell. They have been used in the art as suitable tools for gene delivery in mammalian cells, with most of the viral genes removed. Lentiviral vectors can be used for introducing libraries of synthetic constructs, plasmids, complementary DNAs, short hairpin RNAs, and cis-regulatory elements into many targets, such as embryonic stem cells. In embodiments, lentivirus vector shave the ability to mediate potent transduction and stable expression into dividing and non-dividing cells both in vitro and in vivo. Lentiviruses may have the ability integrate into host chromosomes, and to infect both dividing and non-dividing cells, for example, with sgRNA, gRNA, shRNA and Cas9 (e.g. dCas9, etc.) inserts.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

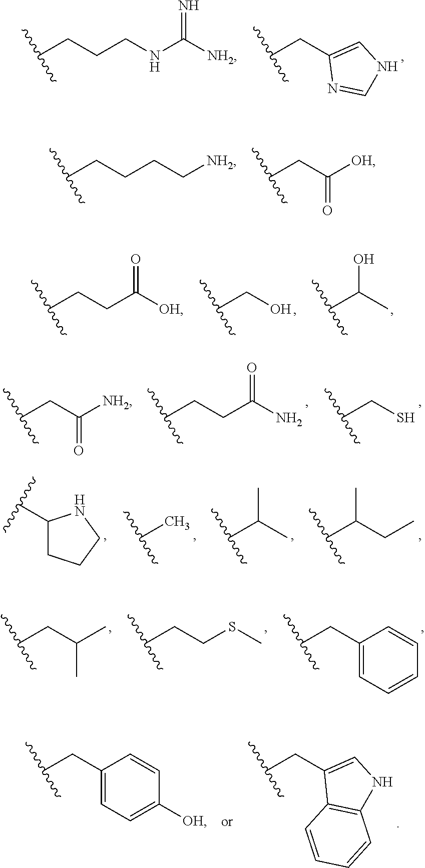

An amino acid residue in a protein “corresponds” to a given residue when it occupies the same essential structural position within the protein as the given residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

For specific proteins described herein, the named protein includes any of the protein's naturally occurring forms, variants or homologs that maintain the protein activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein “Interleukin-15” (“IL-15”) refers to a cytokine that regulates T and NK cell activation and proliferation. This cytokine induces the activation of JAK kinases, as well as the phosphorylation and activation of transcription activators STAT3, STAT5, and STAT6 and may increase the expression of apoptosis inhibitor BCL2L1/BCL-x(L), possibly through the transcription activation activity of STAT6, and thus prevent apoptosis. The terms “IL-15 gene”, “Interleukin-15 gene”, or the like, as used herein refer to the any of the recombinant or naturally-occurring forms of the Interleukin-15 gene or variants or homologs thereof that code for a Interleukin-15 polypeptide capable of maintaining the activity of the Interleukin-15 polypeptide (e.g., within at least 50%, 80%, 90%, 95%, 96%, 97%, 98%, 99% or 100% activity compared to Interleukin-15 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring Interleukin-15e gene. In embodiments, the Interleukin-15 gene is substantially identical to the nucleic acid sequence corresponding to the nucleic acid sequence identified by NCBI Gene ID:3600 or a variant or homolog having substantial identity thereto.

Interferons (IFNs) are a group of signaling proteins made and released by host cells in response to the presence of several viruses. More than twenty distinct IFN genes and proteins have been identified in animals, including humans. They are typically divided among three classes: Type I IFN, Type II IFN, and Type III IFN. IFNs belonging to all three classes are important for fighting viral infections and for the regulation of the immune system. In some aspects, the human interferon gene is a member of the alpha interferon gene cluster on chromosome 9. In some aspects the human IFNα1 gene is NCBI Gene ID: 3439. In some aspects, the human IFNα2 gene is NCBI Gene ID: 3440. In some aspects, the human IFNα2 gene is NCBI Gene ID: 3456. Type I interferon (INF) refers to a group of proteins that are involved in inflammation, immune modulation, tumor cell recognition, and T cell activity. Type I IFNs typically bind to IFN-α receptor on cell surfaces. Type I IFNs include IFN-α and IFN-β.

The terms “IFN-β1”, “IFN-β1 gene”, or the like, as used herein refer to the any of the recombinant or naturally-occurring forms of the IFN-β1 gene, also known as interferon β1 gene, or variants or homologs thereof that code for a IFN-β1 polypeptide capable of maintaining the activity of the IFN-β1 polypeptide (e.g., within at least 50%, 80%, 90%, 95%, 96%, 97%, 98%, 99% or 100% activity compared to IFN-β1 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring IFN-β1 gene. In embodiments, the IFN-β1 gene is substantially identical to the nucleic acid sequence corresponding to the nucleic acid sequence identified by NCBI Gene ID: 3456 or a variant or homolog having substantial identity thereto.

The terms “IFNα1”, “IFNα1 gene”, or the like, as used herein refer to the any of the recombinant or naturally-occurring forms of the IFNα1 gene, also known as interferon α1 gene, or variants or homologs thereof that code for a IFNα1 polypeptide capable of maintaining the activity of the IFNα1 polypeptide (e.g., within at least 50%, 80%, 90%, 95%, 96%, 97%, 98%, 99% or 100% activity compared to IFNα1 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring IFNα1 gene. In embodiments, the IFNα1 gene is substantially identical to the nucleic acid sequence corresponding to the nucleic acid sequence identified by NCBI Gene ID: 3439 or a variant or homolog having substantial identity thereto.

The terms “IFNα2”, “IFNα2 gene”, or the like, as used herein refer to the any of the recombinant or naturally-occurring forms of the IFNα2 gene, also known as interferon α 2 gene, or variants or homologs thereof that code for a IFNα2 polypeptide capable of maintaining the activity of the IFNα2 polypeptide (e.g., within at least 50%, 80%, 90%, 95%, 96%, 97%, 98%, 99% or 100% activity compared to IFNα2 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring IFNα2 gene. In embodiments, the IFNα2 gene is substantially identical to the nucleic acid sequence corresponding to the nucleic acid sequence identified by NCBI Gene ID: 3440 or a variant or homolog having substantial identity thereto. In embodiments, the IFNα2 gene is substantially identical to the nucleic acid sequence corresponding to the nucleic acid sequence identified by NCBI Gene ID: 3456 or a variant or homolog having substantial identity thereto.

The terms “STAT1”, “STAT1 gene”, or the like, as used herein refer to the any of the recombinant or naturally-occurring forms of the STAT1 gene, also known as signal transducer and activator of transcription 1 gene, or variants or homologs thereof that code for a STAT1 polypeptide capable of maintaining the activity of the STAT1 polypeptide (e.g., within at least 50%, 80%, 90%, 95%, 96%, 97%, 98%, 99% or 100% activity compared to STAT1 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring STAT1 gene. In embodiments, the STAT1 gene is substantially identical to the nucleic acid sequence corresponding to the nucleic acid sequence identified by NCBI Gene ID: 6772 or a variant or homolog having substantial identity thereto.

The terms “STAT2 gene”, “STAT2 gene”, or the like, as used herein refer to the any of the recombinant or naturally-occurring forms of the STAT2 gene, also known as signal transducer and activator of transcription 2, or variants or homologs thereof that code for a STAT2 polypeptide capable of maintaining the activity of the STAT2 polypeptide (e.g., within at least 50%, 80%, 90%, 95%, 96%, 97%, 98%, 99% or 100% activity compared to STAT2 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring STAT2 gene. In embodiments, the STAT2 gene is substantially identical to the nucleic acid sequence corresponding to the nucleic acid sequence identified by NCBI Gene ID: 6773 or a variant or homolog having substantial identity thereto.

The terms “IFNAR1”, “IFNAR1 gene”, or the like, as used herein refer to the any of the recombinant or naturally-occurring forms of the IFNAR1 gene, also known as interferon alpha and beta receptor subunit 1 gene, or variants or homologs thereof that code for a IFNAR1 polypeptide capable of maintaining the activity of the IFNAR1 polypeptide (e.g., within at least 50%, 80%, 90%, 95%, 96%, 97%, 98%, 99% or 100% activity compared to IFNAR1 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring IFNAR1 gene. In embodiments, the IFNAR1 gene is substantially identical to the nucleic acid sequence corresponding to the nucleic acid sequence identified by NCBI Gene ID:3454 or a variant or homolog having substantial identity thereto.

The terms “IFNAR2”, “IFNAR2 gene”, or the like, as used herein refer to the any of the recombinant or naturally-occurring forms of the IFNAR2 gene, also known as interferon alpha and beta receptor subunit 2 gene, or variants or homologs thereof that code for a IFNAR2 polypeptide capable of maintaining the activity of the IFNAR2 polypeptide (e.g., within at least 50%, 80%, 90%, 95%, 96%, 97%, 98%, 99% or 100% activity compared to IFNAR2 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring IFNAR2 gene. In embodiments, the IFNAR2 gene is substantially identical to the nucleic acid sequence corresponding to the nucleic acid sequence identified by NCBI Gene ID:3455 or a variant or homolog having substantial identity thereto.

The terms “NKG2D”, “NKG2D gene”, or the like, as used herein refer to the any of the recombinant or naturally-occurring forms of the NKG2D gene, also known as killer cell lectin like receptor K1 gene, or variants or homologs thereof that code for a NKG2D polypeptide capable of maintaining the activity of the NKG2D polypeptide (e.g., within at least 50%, 80%, 90%, 95%, 96%, 97%, 98%, 99% or 100% activity compared to NKG2D polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring NKG2D gene. In embodiments, the NKG2D gene is substantially identical to the nucleic acid sequence corresponding to the nucleic acid sequence identified by NCBI Gene ID:22914 or a variant or homolog having substantial identity thereto.

The terms “NKp46”, “NKp46 gene”, or the like, as used herein refer to the any of the recombinant or naturally-occurring forms of the NKp46 gene, also known as natural cytotoxicity triggering receptor 1 gene, or variants or homologs thereof that code for a NKp46 polypeptide capable of maintaining the activity of the NKp46 polypeptide (e.g., within at least 50%, 80%, 90%, 95%, 96%, 97%, 98%, 99% or 100% activity compared to NKp46 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring NKp46 gene. In embodiments, the NKp46 gene is substantially identical to the nucleic acid sequence corresponding to the nucleic acid sequence identified by NCBI Gene ID: 9437 or a variant or homolog having substantial identity thereto.

The terms “DNAM-1”, “DNAM-1 gene”, or the like, as used herein refer to the any of the recombinant or naturally-occurring forms of the DNAM-1 gene or variants or homologs thereof that code for a DNAM-1 polypeptide capable of maintaining the activity of the DNAM-1 polypeptide (e.g., within at least 50%, 80%, 90%, 95%, 96%, 97%, 98%, 99% or 100% activity compared to DNAM-1 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring DNAM-1 gene. In embodiments, the DNAM-1 gene is substantially identical to the nucleic acid sequence corresponding to the nucleic acid sequence identified by NCBI Gene ID: 10666 or a variant or homolog having substantial identity thereto.

The terms “CD96”, “CD96 gene”, or the like, as used herein refer to the any of the recombinant or naturally-occurring forms of the CD96 gene or variants or homologs thereof that code for a CD96 polypeptide capable of maintaining the activity of the CD96 polypeptide (e.g., within at least 50%, 80%, 90%, 95%, 96%, 97%, 98%, 99% or 100% activity compared to CD96 polypeptide). In some aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring CD96 gene. In embodiments, the CD96 gene is substantially identical to the nucleic acid sequence corresponding to the nucleic acid sequence identified by NCBI Gene ID: 10225 or a variant or homolog having substantial identity thereto.

“Nuclease” and “endonuclease” are used interchangeably herein to mean an enzyme which possesses endonucleolytic catalytic activity for nucleic acid (e.g., polynucleotide) cleavage. The term includes site-specific endonucleases such as, designer zinc fingers, transcription activator-like effectors (TALEs), homing meganucleases, and site-specific endonucleases of clustered, regularly interspaced, short palindromic repeat (CRISPR) systems such as, e.g., Cas proteins.

A “ribonucleoprotein complex,” or “ribonucleoprotein particle” as provided herein refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. Non-limiting examples of ribonucleoproteins include ribosomes, telomerase, RNAseP, hnRNP, CRISPR associated protein 9 (Cas9) and small nuclear RNPs (snRNPs). The ribonucleoprotein may be an enzyme. In embodiments, the ribonucleoprotein is an endonuclease. Thus, in embodiments, the ribonucleoprotein complex includes an endonuclease and a ribonucleic acid. In embodiments, the endonuclease is a CRISPR associated

The term “site-specific modifying polypeptide” or “RNA-binding site-specific modifying polypeptide” as used herein is a polypeptide that binds RNA and is targeted to a specific DNA sequence, such as a Cas9 polypeptide, a Cpf1 polypeptide, a C2c1 polypeptide, a C2c2 polypeptide, or a C2c3 polypeptide. A site-specific modifying polypeptide as described herein is targeted to a specific DNA sequence by the RNA molecule to which it is bound (e.g., sgRNA). The RNA molecule (e.g., sgRNA) includes a sequence that binds, hybridizes to, or is complementary to a target sequence within the target DNA, thus targeting the bound polypeptide to a specific location within the target DNA (the target sequence). This RNA molecule can be a single guide RNA (sgRNA). In embodiments, the sgRNA is about 10 to about 30 nucleotides long. In embodiments, the sgRNA is 10 to 30 nucleotides long. In embodiments, the sgRNA is 10 to 40 nucleotides long. In embodiments, the sgRNA is 10 to 50 nucleotides long. In embodiments, the sgRNA is about 10, 11, 12, 13, 14, 15, 16, 17, 18, 19, 20, 21, 22, 23, 24, 25, 26, 27, 28, 29, or 30 nucleotides long. In embodiments, the sgRNA is 10, 11 12, 13, 14, 15, 16, 17, 18, 19, 20, 21, 22, 23, 24, 25, 26, 27, 28, 29, or 30 nucleotides long. In embodiments, the sgRNA is 10 nucleotides long. In embodiments, the sgRNA is 11 nucleotides long. In embodiments, the sgRNA is 12 nucleotides long. In embodiments, the sgRNA is 13 nucleotides long. In embodiments, the sgRNA is 14 nucleotides long. In embodiments, the sgRNA is 15 nucleotides long. In embodiments, the sgRNA is 16 nucleotides long. In embodiments, the sgRNA is 17 nucleotides long. In embodiments, the sgRNA is 18 nucleotides long. In embodiments, the sgRNA is 19 nucleotides long. In embodiments, the sgRNA is 20 nucleotides long. In embodiments, the sgRNA is 21 nucleotides long. In embodiments, the sgRNA is 22 nucleotides long. In embodiments, the sgRNA is 23 nucleotides long. In embodiments, the sgRNA is 24 nucleotides long. In embodiments, the sgRNA is 25 nucleotides long. In embodiments, the sgRNA is 26 nucleotides long. In embodiments, the sgRNA is 27 nucleotides long. In embodiments, the sgRNA is 28 nucleotides long. In embodiments, the sgRNA is 29 nucleotides long. In embodiments, the sgRNA is 30 nucleotides long. In some cases, the sgRNAs can be selected to inhibit transcription of target loci (e.g., targeted to optimized human CRISPRi target sites), activate transcription of target loci (e.g., targeted to optimized human CRISPRa target sites.

In embodiments, the site-specific modifying polypeptide can be nuclease deficient. In embodiments, the Cas9 protein can be a nuclease deficient sgRNA-mediated nuclease (dCas9). dCas9 refers to a Cas9 protein, or functional fragment thereof, that has decreased nuclease activity relative to a Cas9 protein found in nature. In embodiments, the dCas9 is a mutant form of Cas9 whose endonuclease activity is reduced or removed through mutations (e.g., point mutations) in its endonuclease domains. In embodiments, dCas9 is used in CRISPR systems along with gRNAs (sgRNA) to target specific genes or nucleotides complementary to the gRNA with PAM sequences. As is known in the art, the sgRNA provides targeted nucleic acid binding activity as part of a Cas9 and/or dCas9 system. Cas9 ordinarily has 2 endonuclease domains called the RuvC and HNH domains. In embodiments, the point mutations D10A and H840A deactivates the normal Cas9 endonuclease activity. Although dCas9 lacks endonuclease activity relative to Cas9, it is still capable of binding to its guide RNA (sgRNA) and, therefore, the DNA strand that is being targeted. In embodiments, dCas9 is capable of attenuating or blocking transcription of the targeted gene. In embodiments, the ability of dCas9 to bind DNA can also be exploited for gene activation by modification of dCas9 (e.g., the N and C terminus of the protein) to attach transcriptional activators or recruit transcriptional activators. In embodiment, the ability of dCad9 to bind DNA can also be exploited for gene inactivation by modification of dCas9 (e.g., the N and C terminus of the protein) to attach transcriptional inactivators or recruit transcriptional in activators. In embodiments, a dCas9 includes a dCas9 domain fused to a transcriptional modulator. This transcriptional modulator can be, e.g., a DNA methyltransferase. In embodiments, the target DNA is a gene (target gene) present in the same cell as the Cas9 or dCas9 protein. In embodiments, the dCas9 has substantially no detectable endonuclease (e.g., endodeoxyribonuclease) activity. In embodiments, dCas9 includes the amino acid sequence of SEQ ID NO:20. In embodiments, dCas9 has the amino acid sequence of SEQ ID NO:20. In embodiments, dCas9 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:20. In embodiments, dCas9 has an amino acid sequence that has at least 75% sequence identity to SEQ ID NO:20. In aspects, dCas9 has an amino acid sequence that has at least 80% sequence identity to SEQ ID NO:20. In embodiments, dCas9 has an amino acid sequence that has at least 85% sequence identity to SEQ ID NO:20. In aspects, dCas9 has an amino acid sequence that has at least 90% sequence identity to SEQ ID NO:20. In embodiments, dCas9 has an amino acid sequence that has at least 95% sequence identity to SEQ ID NO:20.

The term “VP64” or “VP64 protein” as provided herein includes any of the recombinant or naturally-occurring forms of Tegument protein VP16 (VP64), also known as Alpha trans-inducing protein, Alpha-TIF, or variants or homologs thereof that maintain VP64 protein activity (e.g. within at least 50%, 80%, 90%, 95%, 96%, 97%, 98%, 99% or 100% activity compared to VP64 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VP64 protein polypeptide. In embodiments, VP64 protein is the protein as identified by the UniProt reference number P06492, or a variant, homolog or functional fragment thereof.

The term “Krüppel associated box domain”, “KRAB domain” or “KRAB” as provided herein refers to a category of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, 2017; Lambert et al. The human transcription factors, Cell 172, 2018; Gilbert et al., Cell (2013); and Gilbert et al., Cell (2014), all of which are incorporated herein by reference in their entirety. In aspects, the KRAB domain is a KRAB domain of Kox 1. In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KRAB domain polypeptide. In embodiments, KRAB protein is the protein as identified by the UniProt reference number 014913, or a variant, homolog or functional fragment thereof.

The term “IL-15” or “IL-15 protein” as provided herein includes any of the recombinant or naturally-occurring forms of the interleukin-15 (IL-15), or variants or homologs thereof that maintain IL-15 protein activity (e.g. within at least 50%, 80%, 90%, 95%, 96%, 97%, 98%, 99% or 100% activity compared to IL-15). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 polypeptide. In embodiments, IL-15 is the protein as identified by the UniProt reference number P40933 or a variant or homolog having substantial identity thereto.

The term “INF-γ” or “INF-γ protein” as provided herein includes any of the recombinant or naturally-occurring forms of the interferon gamma (INF-γ), or variants or homologs thereof that maintain INF-7 protein activity (e.g. within at least 50%, 80%, 90%, 95%, 96%, 97%, 98%, 99% or 100% activity compared to INF-γ). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring INF-γ polypeptide. In embodiments, INF-7 is the protein as identified by the UniProt reference number P01579 or a variant or homolog having substantial identity thereto.

The term “EGFR protein” or “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

The term “Her2 protein” or “Her2” as used herein includes any of the recombinant or naturally-occurring forms of Receptor tyrosine-protein kinase erbB-2, also known as CD340 (cluster of differentiation 340), proto-oncogene Neu, Erbb2 (rodent), or ERBB2 (human), or variants or homologs thereof that maintain Her2 activity (e.g. within at least 50%, 80%, 90%, 95%, 96%, 97%, 98%, 99% or 100% activity compared to Her2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Her2 protein. In embodiments, the Her2 protein is substantially identical to the protein identified by the UniProt reference number P04626 or a variant or homolog having substantial identity thereto.

The term “transcriptional activator” and the like refer, in the usual and customary sense, to a polypeptide or protein that increases the rate of transcription of genetic information from DNA to messenger RNA, by binding to a specific DNA sequence, when compared to a control or baseline expression level (e.g., in the absence of the transcriptional activator). Non-limiting examples of transcriptional activators are the transactivator domain of the Herpes simplex viral protein 16 (VP16), multiple copies of VP16 (4 copies of VP16 (VP64) or 10 copies of VP16 (VP160)), the transactivator domain of nuclear factor kappa B (p65), and the Epstein-Barr virus R transactivator (Rta).

The term “transcriptional inactivator” or “transcriptional repressor” and the like refer, in the usual and customary sense, to a polypeptide or protein that decreases the rate of transcription of genetic information from DNA to messenger RNA, by binding to a specific DNA sequence, when compared to a control or baseline expression level (e.g., in the absence of the transcriptional activator). Non-limiting examples of transcriptional inactivators are the KRAB (Krüppel associated box) domain of Kox1, the WRPW domain of Hes1, the CS (Chromo Shadow) domain of HP1α, and the SID4X domain.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system. The C2c1 (“Class 2 candidate 1”) enzyme is a Class II type V-B enzyme. The C2c2 (“Class 2 candidate 2”) enzyme is a Class II type V1-A enzyme. The C2c (“Class 2 candidate 3”) enzyme is a Class 11 type V-C enzyme.

Thus, a “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g., within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase”. In embodiments, Cas9 is an RNA-guided DNA endonuclease enzyme that binds a CRISPR (clustered regularly interspaced short palindromic repeats) nucleic acid sequence. In embodiments, the CRISPR nucleic acid sequence is a prokaryotic nucleic acid sequence. In embodiments, the Cas9 nuclease from Streptococcus pyogenes is targeted to genomic DNA by a synthetic guide RNA consisting of a 20-nt guide sequence and a scaffold. The guide sequence base-pairs with the DNA target, directly upstream of a requisite 5′-NGG protospacer adjacent motif (PAM), and Cas9 mediates a double-stranded break (DSB) about 3-base pair upstream of the PAM. In embodiments, the CRISPR nuclease from Streptococcus aureus is targeted to genomic DNA by a synthetic guide RNA consisting of a 21-23-nt guide sequence and a scaffold. The guide sequence base-pairs with the DNA target, directly upstream of a requisite 5′-NNGRRT protospacer adjacent motif (PAM), and Cas9 mediates a double-stranded break (DSB) about 3-base pair upstream of the PAM.

By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In some embodiments, a complex comprising a guide RNA and a site-specific modifying enzyme is used for targeted double-stranded DNA cleavage.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

An “inhibitor” refers to a compound (e.g., compounds described herein) that reduces activity when compared to a control, such as absence of the compound or a compound with known inactivity.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “detectable agent” or “detectable moiety” is a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y ⁸⁹Sr, ⁸⁹Zr, ⁹⁴Tc, ⁹⁴Tc, ^{99m}Tc, ⁹⁹Mo, ¹⁰⁵Pd, ¹⁰⁵Ph, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Lr, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., a protein associated disease, a cancer (e.g., leukemia)) means that the disease (e.g., cancer) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g., humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, breast cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The term “healthy patient” or “healthy subject” as used herein refers to a subject that does not have cancer. Provided herein are methods of treating or preventing infectious disease in a cancer patient. In embodiments, the cancer is leukemia. As used herein, the healthy subject does not have leukemia. In embodiments, the healthy subject does not have ALL. In embodiments, the healthy subject does not have an infectious disease.

A “effective amount” or “therapeutically effective amount” are used interchangeably and refer to an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). For any composition (e.g. NK cell composition) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Allogeneic” is used in accordance with its plain and ordinary meaning and includes cells or tissues derived from different individuals of the same species. The cells or tissues may be sufficiently unlike genetically to interact antigenically. In embodiments, the allogeneic cells are allogeneic NK cells. Thus, the term “allogeneic transplant” or “allogeneic transfusion” refers to the transfer of biological material (e.g. NK cells) to a recipient from a genetically non-identical donor of the same species.

“Autologous” is used in accordance with its plain and ordinary meaning and includes cells or tissues derived from the same individual. In embodiments, the autologous cells are autologous NK cells. An autolous NK cell may be taken from an individual and genetically modified (e.g. nucleic acid integrated into NK cell genome) before being put back into the same individual.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g., mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g., mice) and from humans.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

As used herein, the terms “natural killer cells” and “NK cells” are used in accordance with their plain ordinary meaning and refer to a type of cytotoxic lymphocyte involved in the innate immune system. The role NK cells play is typically analogous to that of cytotoxic T cells in the vertebrate adaptive immune response. NK cells may provide rapid responses to virus-infected cells, acting at around 3 days after infection, and respond to tumor formation. Typically, immune cells detect major histocompatibility complex (MHC) presented on infected cell surfaces, triggering cytokine release, causing lysis or apoptosis. NK cells typically have the ability to recognize stressed cells in the absence of antibodies and MHC, allowing for a much faster immune reaction. In embodiments, NK cells are identified by the presence of CD56 and the absence of CD3. NK cells may be capable of recognizing and killing stressed cells in the absence of antibodies and MHC.

A “regulatory T cell” or “suppressor T cell” is a lymphocyte which modulates the immune system, maintains tolerance to self-antigens, and prevents autoimmune disease.

The terms “virus” or “virus particle” are used according to its plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell. A “replication-competent” virus as provided herein refers to a virus (chimeric poxvirus) that is capable of replicating in a cell (e.g., a cancer cell). Similarly, an “oncolytic virus” as referred to herein, is a virus that is capable of infecting and killing cancer cells. As the infected cancer cells are destroyed by oncolysis, they release new infectious virus particles or virions to help destroy the remaining tumor. In embodiments, the chimeric poxvirus is able to replicate in a cancer cell. In embodiments, the chimeric poxvirus does not detectably replicate in a healthy cell relative to a standard control. In embodiments, the chimeric poxvirus provided herein has an increased oncolytic activity compared to its parental virus. In embodiments, the oncolytic activity (ability to induce cell death in an infected cell) is more than 1.5, 2, 3, 4, 5, 6, 7, 8, 9, 10, 100, 10000, 10000 times increased compared to the oncolytic activity of a parental virus (one of the viruses used to form the chimeric virus provided herein).

The terms “lentivirus” as used herein refers to a viral species belonging to the family of Retroviridae that include human immunodeficiency virus I (HIV). Lentivirus are a family of viruses that are responsible for notable diseases like AIDS, which infect by inserting DNA into their host cells' genome. Many such viruses have been the basis of research using viruses in gene therapy, but the lentivirus is unique in its ability to infect non-dividing cells, and therefore has a wider range of potential applications. Lentiviruses can become endogenous (ERV), integrating their genome into the host germline genome, so that the virus is henceforth inherited by the host's descendants. To be effective in gene therapy, there must be insertion, alteration and/or removal of host cell genes.

### Compositions

Provided herein, inter alia, are compositions including engineered Natural Killer (NK) cells. The engineered NK cells provided herein include integrated nucleic acid sequences encoding catalytically inactive Cas9 (e.g. dCas9). In embodiments, constitutive expression of dCas9 allows for the NK cells to express proteins (e.g. interferons, IL-15, etc.) that increase NK cell cytotoxic effects and reduce NK cell exhaustion. Thus, in an aspect is provided a Natural Killer (NK) cell including an integrated nucleic acid sequence encoding a catalytically inactive Cas9 (dCas9) protein.

In embodiments, the NK cell is obtained by transfection or transduction of an exogenous nucleic acid encoding the catalytically inactive Cas9 (dCas9) protein. In embodiments, the NK cell is obtained by transfection of an exogenous nucleic acid encoding the catalytically inactive Cas9 (dCas9) protein. In embodiments, the NK cell is obtained by transduction of an exogenous nucleic acid encoding the catalytically inactive Cas9 (dCas9) protein. In embodiments, dCas9 includes the amino acid sequence of SEQ ID NO:20. In embodiments, dCas9 has the amino acid sequence of SEQ ID NO:20. In embodiments, dCas9 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:20. In embodiments, dCas9 has an amino acid sequence that has at least 75% sequence identity to SEQ ID NO:20. In aspects, dCas9 has an amino acid sequence that has at least 80% sequence identity to SEQ ID NO:20. In embodiments, dCas9 has an amino acid sequence that has at least 85% sequence identity to SEQ ID NO:20. In aspects, dCas9 has an amino acid sequence that has at least 90% sequence identity to SEQ ID NO:20. In embodiments, dCas9 has an amino acid sequence that has at least 95% sequence identity to SEQ ID NO:20.

In embodiments, the exogenous nucleic acid ending the dCas9 protein is present within a vector suitable for integrating nucleic acids into a host genome. For example, a viral vector may be used which inserts nucleic acid sequences within the vector into the host genome. The host genome may then express a protein encoded by the integrated nucleic acid sequence. Thus, in embodiments, the exogenous nucleic acid encoding the catalytically inactive Cas9 (dCas9) protein is present within a lentiviral vector. In embodiments, the integrated nucleic acid sequence encoding a catalytically inactive Cas9 (dCas9) protein includes a lentiviral nucleic acid sequence.

In embodiments, the integrated nucleic acid includes from about 100 to about 10,000 base pairs. In embodiments, the integrated nucleic acid includes from about 500 to about 10,000 base pairs. In embodiments, the integrated nucleic acid includes from about 1000 to about 10,000 base pairs. In embodiments, the integrated nucleic acid includes from about 1500 to about 10,000 base pairs. In embodiments, the integrated nucleic acid includes from about 1000 to about 10,000 base pairs. In embodiments, the integrated nucleic acid includes from about 1500 to about 10,000 base pairs. In embodiments, the integrated nucleic acid includes from about 2000 to about 10,000 base pairs. In embodiments, the integrated nucleic acid includes from about 2500 to about 10,000 base pairs. In embodiments, the integrated nucleic acid includes from about 3000 to about 10,000 base pairs. In embodiments, the integrated nucleic acid includes from about 3500 to about 10,000 base pairs. In embodiments, the integrated nucleic acid includes from about 4000 to about 10,000 base pairs. In embodiments, the integrated nucleic acid includes from about 4500 to about 10,000 base pairs. In embodiments, the integrated nucleic acid includes from about 5000 to about 10,000 base pairs. In embodiments, the integrated nucleic acid includes from about 5500 to about 10,000 base pairs. In embodiments, the integrated nucleic acid includes from about 6000 to about 10,000 base pairs. In embodiments, the integrated nucleic acid includes from about 6500 to about 10,000 base pairs. In embodiments, the integrated nucleic acid includes from about 7000 to about 10,000 base pairs. In embodiments, the integrated nucleic acid includes from about 7500 to about 10,000 base pairs. In embodiments, the integrated nucleic acid includes from about 8000 to about 10,000 base pairs. In embodiments, the integrated nucleic acid includes from about 8500 to about 10,000 base pairs. In embodiments, the integrated nucleic acid includes from about 9000 to about 10,000 base pairs. In embodiments, the integrated nucleic acid includes from about 9500 to about 10,000 base pairs.

In embodiments, the integrated nucleic acid includes from about 1,000 to about 9,500 base pairs. In embodiments, the integrated nucleic acid includes from about 1,000 to about 9,000 base pairs. In embodiments, the integrated nucleic acid includes from about 1,000 to about 8,500 base pairs. In embodiments, the integrated nucleic acid includes from about 1,000 to about 8,000 base pairs. In embodiments, the integrated nucleic acid includes from about 1,000 to about 7,500 base pairs. In embodiments, the integrated nucleic acid includes from about 1,000 to about 7,000 base pairs. In embodiments, the integrated nucleic acid includes from about 1,000 to about 6,500 base pairs. In embodiments, the integrated nucleic acid includes from about 1,000 to about 6,000 base pairs. In embodiments, the integrated nucleic acid includes from about 1,000 to about 5,500 base pairs. In embodiments, the integrated nucleic acid includes from about 1,000 to about 5,000 base pairs. In embodiments, the integrated nucleic acid includes from about 1,000 to about 4,500 base pairs. In embodiments, the integrated nucleic acid includes from about 1,000 to about 4,000 base pairs. In embodiments, the integrated nucleic acid includes from about 1,000 to about 3,500 base pairs. In embodiments, the integrated nucleic acid includes from about 1,000 to about 3,000 base pairs. In embodiments, the integrated nucleic acid includes from about 1,000 to about 2,500 base pairs. In embodiments, the integrated nucleic acid includes from about 1,000 to about 2,000 base pairs. In embodiments, the integrated nucleic acid includes from about 1,000 to about 1,500 base pairs. In embodiments, the integrated nucleic acid includes from about 100, 500, 1000, 1500, 2000, 2500, 3000, 3500, 4000, 4500, 5000, 5500, 6000, 6500, 7000, 7500, 8000, 8500, 9000, 9500, or 10,000 base pairs.

In embodiments, the integrated nucleic acid includes from about 150 to about 1200 base pairs. In embodiments, the integrated nucleic acid includes from about 300 to about 1200 base pairs. In embodiments, the integrated nucleic acid includes from about 450 to about 1200 base pairs. In embodiments, the integrated nucleic acid includes from about 600 to about 1200 base pairs. In embodiments, the integrated nucleic acid includes from about 750 to about 1200 base pairs. In embodiments, the integrated nucleic acid includes from about 900 to about 1200 base pairs. In embodiments, the integrated nucleic acid includes from about 1050 to about 1200 base pairs.

In embodiments, the integrated nucleic acid includes from about 150 to about 1050 base pairs. In embodiments, the integrated nucleic acid includes from about 150 to about 900 base pairs. In embodiments, the integrated nucleic acid includes from about 150 to about 750 base pairs. In embodiments, the integrated nucleic acid includes from about 150 to about 600 base pairs. In embodiments, the integrated nucleic acid includes from about 150 to about 450 base pairs. In embodiments, the integrated nucleic acid includes from about 150 to about 300 base pairs. In embodiments, the integrated nucleic acid includes about 150, 300, 450, 600, 750, 900, 1050 or 1200 base pairs.

In embodiments, the NK cell further includes a nucleic acid encoding an sgRNA sequence. In embodiments, the sgRNA sequence includes the nucleic acid sequence of SEQ ID NO:3, 4, 5, 6, 7, 8, 9, 10, 15, 16, 17, or 18. In embodiments, the sgRNA sequence includes the nucleic acid sequence of SEQ ID NO:3. In embodiments, the sgRNA sequence includes the nucleic acid sequence of SEQ ID NO:4 In embodiments, the sgRNA sequence includes the nucleic acid sequence of SEQ ID NO: 5. In embodiments, the sgRNA sequence includes the nucleic acid sequence of SEQ ID NO: 6. In embodiments, the sgRNA sequence includes the nucleic acid sequence of SEQ ID NO: 7. In embodiments, the sgRNA sequence includes the nucleic acid sequence of SEQ ID NO: 8. In embodiments, the sgRNA sequence includes the nucleic acid sequence of SEQ ID NO:9. In embodiments, the sgRNA sequence includes the nucleic acid sequence of SEQ ID NO: 10. In embodiments, the sgRNA sequence includes the nucleic acid sequence of SEQ ID NO: 15. In embodiments, the sgRNA sequence includes the nucleic acid sequence of SEQ ID NO: 16. In embodiments, the sgRNA sequence includes the nucleic acid sequence of SEQ ID NO: 17. In embodiments, the sgRNA sequence includes the nucleic acid sequence of SEQ ID NO: 18. In embodiments, the nucleic acid encoding the sgRNA sequence is integrated into the genome of the NK cell.

In embodiments, the NK cell constitutively expresses the dCas9 protein. For example, an NK cell that constitutively expresses dCas9 protein may continuously transcribe the nucleic acid sequence encoding the dCas9 protein. Thus, in embodiments, the NK cell that constitutively expresses dCas9 continuously produces mRNA encoding the dCas9 protein. In embodiments, the NK cell that constitutively expresses dCas9 continuously produces the dCas9 protein.

In embodiments, the NK cell further constitutively expresses a detectable protein. For example, the expression of the detectable protein may be indicative of whether dCas9 is expressed. In embodiments, the detectable protein is GFP, EGFP or RFP. In embodiments, the detectable protein is GFP. In embodiments, the detectable protein is EGFP. In embodiments, the detectable protein is RFP. In embodiments, the NK cell constitutively expresses dCas9 and GFP. In embodiments, the detectable protein may be used to isolate the NK cells (e.g. engineered NK cells) from a population of cells (e.g. non-engineered NK cells). In embodiments, the expression of GFP is dependent on the expression of dCas9. In embodiments, the expression of GFP is used as a indicator of the expression of dCas9. In embodiments, the NK cell constitutively expresses dCas9 and EGFP. In embodiments, the detectable protein may be used to isolate the NK cells (e.g. engineered NK cells) from a population of cells (e.g. non-engineered NK cells). In embodiments, the expression of EGFP is dependent on the expression of dCas9. In embodiments, the expression of EGFP is used as a indicator of the expression of dCas9. In embodiments, the NK cell constitutively expresses dCas9 and RFP. In embodiments, the detectable protein may be used to isolate the NK cells (e.g. engineered NK cells) from a population of cells (e.g. non-engineered NK cells). In embodiments, the expression of RFP is dependent on the expression of dCas9. In embodiments, the expression of RFP is used as a indicator of the expression of dCas9.

In another aspect is provided a substantially pure population of Natural Killer (NK) cells, wherein each of the NK cells includes an integrated nucleic acid sequence encoding a Cas9 protein. In another aspect is provided a substantially pure population of Natural Killer (NK) cells, wherein at least 80% of the NK cells include an integrated nucleic acid sequence encoding a Cas9 protein. In another aspect is provided a substantially pure population of Natural Killer (NK) cells, wherein each of the NK cells includes an integrated nucleic acid sequence encoding a catalytically inactive Cas9 (dCas9) protein. In another aspect is provided a substantially pure population of Natural Killer (NK) cells, wherein at least 80% of the NK cells include an integrated nucleic acid sequence encoding a catalytically inactive Cas9 (dCas9) protein. A “substantially pure” population of NK cells refers to a population of cells wherein at least about 10% to 100% of cells in the population are NK cells provided herein including embodiments thereof (e.g. NK cells including an integrated nucleic acid sequence encoding a Cas9 protein or a dCas9 protein). In embodiments, the substantially pure population of NK cells provided herein including embodiments thereof is generated from a population of cells including about 0.1-2% of the NK cell (e.g. NK cells including an integrated nucleic acid sequence encoding a Cas9 protein or a dCas9 protein) provided herein including embodiments thereof.

In embodiments, a substantially pure population includes about 10% to 100% of NK cells provided herein including embodiments thereof (e.g. NK cells including an integrated nucleic acid sequence encoding a Cas9 protein or a dCas9 protein). In embodiments, a substantially pure population includes about 15% to 100% of NK cells provided herein including embodiments thereof. In embodiments, a substantially pure population includes about 20% to 100% of NK cells provided herein including embodiments thereof. In embodiments, a substantially pure population includes about 25% to 100% of NK cells provided herein including embodiments thereof. In embodiments, a substantially pure population includes about 30% to 100% of NK cells provided herein including embodiments thereof. In embodiments, a substantially pure population includes about 35% to 100% of NK cells provided herein including embodiments thereof. In embodiments, a substantially pure population includes about 40% to 100% of NK cells provided herein including embodiments thereof. In embodiments, a substantially pure population includes about 45% to 100% of NK cells provided herein including embodiments thereof. In embodiments, a substantially pure population includes about 50% to 100% of NK cells provided herein including embodiments thereof. In embodiments, a substantially pure population includes about 55% to 100% of NK cells provided herein including embodiments thereof. In embodiments, a substantially pure population includes about 60% to 100% of NK cells provided herein including embodiments thereof. In embodiments, a substantially pure population includes about 65% to 100% of NK cells provided herein including embodiments thereof. In embodiments, a substantially pure population includes about 70% to 100% of NK cells provided herein including embodiments thereof. In embodiments, a substantially pure population includes about 75% to 100% of NK cells provided herein including embodiments thereof. In embodiments, a substantially pure population includes about 80% to 100% of NK cells provided herein including embodiments thereof. In embodiments, a substantially pure population includes about 85% to 100% of NK cells provided herein including embodiments thereof. In embodiments, a substantially pure population includes about 90% to 100% of NK cells provided herein including embodiments thereof. In embodiments, a substantially pure population includes about 95% to 100% of NK cells provided herein including embodiments thereof.

In embodiments, a substantially pure population includes about 10% to 95% of NK cells provided herein including embodiments thereof (e.g. NK cells including an integrated nucleic acid sequence encoding a Cas9 protein or a dCas9 protein). In embodiments, a substantially pure population includes about 10% to 90% of NK cells provided herein including embodiments thereof. In embodiments, a substantially pure population includes about 10% to 85% of NK cells provided herein including embodiments thereof. In embodiments, a substantially pure population includes about 10% to 80% of NK cells provided herein including embodiments thereof. In embodiments, a substantially pure population includes about 10% to 75% of NK cells provided herein including embodiments thereof. In embodiments, a substantially pure population includes about 10% to 70% of NK cells provided herein including embodiments thereof. In embodiments, a substantially pure population includes about 10% to 65% of NK cells provided herein including embodiments thereof. In embodiments, a substantially pure population includes about 10% to 60% of NK cells provided herein including embodiments thereof. In embodiments, a substantially pure population includes about 10% to 55% of NK cells provided herein including embodiments thereof. In embodiments, a substantially pure population includes about 10% to 50% of NK cells provided herein including embodiments thereof. In embodiments, a substantially pure population includes about 10% to 45% of NK cells provided herein including embodiments thereof. In embodiments, a substantially pure population includes about 10% to 40% of NK cells provided herein including embodiments thereof. In embodiments, a substantially pure population includes about 10% to 35% of NK cells provided herein including embodiments thereof. In embodiments, a substantially pure population includes about 10% to 30% of NK cells provided herein including embodiments thereof. In embodiments, a substantially pure population includes about 10% to 25% of NK cells provided herein including embodiments thereof. In embodiments, a substantially pure population includes about 10% to 20% of NK cells provided herein including embodiments thereof. In embodiments, a substantially pure population includes about 10% to 15% of NK cells provided herein including embodiments thereof. In embodiments, a substantially pure population includes at about 10%, 15%, 20%, 25%, 30%, 35%, 40%, 45%, 50%, 55%, 60%, 65%, 70%, 75%, 80%, 85%, 90%, 95%, or 100% of the NK cells provided herein including embodiments thereof.

In embodiments, the within the population of of substantially pure NK cells includes less than 15% un-transduced NK cells (e.g. NK cells including an integrated nucleic acid sequence encoding a Cas9 protein or a dCas9 protein). In embodiments, less than 14% of the cells are un-transduced NK cells. In embodiments, less than 13% of the cells are un-transduced NK cells. In embodiments, less than 12% of the cells are un-transduced NK cells. In embodiments, less than 11% of the cells are un-transduced NK cells. In embodiments, less than 10% of the cells are un-transduced NK cells. In embodiments, less than 9% of the cells are un-transduced NK cells. In embodiments, less than 8% of the cells are un-transduced NK cells. In embodiments, less than 7% of the cells are un-transduced NK cells. In embodiments, less than 6% of the cells are un-transduced NK cells. In embodiments, less than 5% of the cells are un-transduced NK cells. In embodiments, less than 4% of the cells are un-transduced NK cells. In embodiments, less than 3% of the cells are un-transduced NK cells. In embodiments, less than 2% of the cells are un-transduced NK cells. In embodiments, less than 1% of the cells are un-transduced NK cells. In embodiments, less than 0.9% of the cells are un-transduced NK cells. In embodiments, less than 0.8% of the cells are un-transduced NK cells. In embodiments, less than 0.7% of the cells are un-transduced NK cells. In embodiments, less than 0.6% of the cells are un-transduced NK cells. In embodiments, less than 0.5% of the cells are un-transduced NK cells. In embodiments, less than 0.4% of the cells are un-transduced NK cells. In embodiments, less than 0.3% of the cells are un-transduced NK cells. In embodiments, less than 0.2% of the cells are un-transduced NK cells. In embodiments, less than 0.1% of the cells are un-transduced NK cells. In embodiments, 0% of the cells are un-transduced NK cells.

In embodiments, the term “substantially pure” in reference to a particular population of Natural Killer (NK) cells as used herein, refers to a population of NK cells wherein at least 85% of the cells are of the particular type of NK cells referred to therein (e.g., NK cells including an integrated nucleic acid sequence encoding a Cas9 protein). In embodiments, at least 86% of the cells are of the particular type of NK cells referred to therein (e.g., NK cells including an integrated nucleic acid sequence encoding a Cas9 protein). In embodiments, at least 87% of the cells are of the particular type of NK cells referred to therein (e.g., NK cells including an integrated nucleic acid sequence encoding a Cas9 protein). In embodiments, at least 88% of the cells are of the particular type of NK cells referred to therein (e.g., NK cells including an integrated nucleic acid sequence encoding a Cas9 protein). In embodiments, at least 89% of the cells are of the particular type of NK cells referred to therein (e.g., NK cells including an integrated nucleic acid sequence encoding a Cas9 protein). In embodiments, at least 90% of the cells are of the particular type of NK cells referred to therein (e.g., NK cells including an integrated nucleic acid sequence encoding a Cas9 protein). In embodiments, at least 91% of the cells are of the particular type of NK cells referred to therein (e.g., NK cells including an integrated nucleic acid sequence encoding a Cas9 protein). In embodiments, at least 92% of the cells are of the particular type of NK cells referred to therein (e.g., NK cells including an integrated nucleic acid sequence encoding a Cas9 protein). In embodiments, at least 93% of the cells are of the particular type of NK cells referred to therein (e.g., NK cells including an integrated nucleic acid sequence encoding a Cas9 protein). In embodiments, at least 94% of the cells are of the particular type of NK cells referred to therein (e.g., NK cells including an integrated nucleic acid sequence encoding a Cas9 protein). In embodiments, at least 95% of the cells are of the particular type of NK cells referred to therein (e.g., NK cells including an integrated nucleic acid sequence encoding a Cas9 protein). In embodiments, at least 96% of the cells are of the particular type of NK cells referred to therein (e.g., NK cells including an integrated nucleic acid sequence encoding a Cas9 protein). In embodiments, 97% of the cells are of the particular type of NK cells referred to therein (e.g., NK cells including an integrated nucleic acid sequence encoding a Cas9 protein). In embodiments, at least 98% of the cells are of the particular type of NK cells referred to therein (e.g., NK cells including an integrated nucleic acid sequence encoding a Cas9 protein). In embodiments, at least 99% of the cells are of the particular type of NK cells referred to therein (e.g., NK cells including an integrated nucleic acid sequence encoding a Cas9 protein). In embodiments, 100% of the cells are of the particular type of NK cells referred to therein (e.g., NK cells including an integrated nucleic acid sequence encoding a Cas9 protein). In embodiments, the population of NK cells comprises less than 15% of the cells are un-transduced NK cells. In embodiments, less than 14% of the cells are un-transduced NK cells. In embodiments, less than 13% of the cells are un-transduced NK cells. In embodiments, less than 12% of the cells are un-transduced NK cells. In embodiments, less than 11% of the cells are un-transduced NK cells. In embodiments, less than 10% of the cells are un-transduced NK cells. In embodiments, less than 9% of the cells are un-transduced NK cells. In embodiments, less than 8% of the cells are un-transduced NK cells. In embodiments, less than 7% of the cells are un-transduced NK cells. In embodiments, less than 6% of the cells are un-transduced NK cells. In embodiments, less than 5% of the cells are un-transduced NK cells. In embodiments, less than 4% of the cells are un-transduced NK cells. In embodiments, less than 3% of the cells are un-transduced NK cells. In embodiments, less than 2% of the cells are un-transduced NK cells. In embodiments, less than 1% of the cells are un-transduced NK cells. In embodiments, less than 0.9% of the cells are un-transduced NK cells. In embodiments, less than 0.8% of the cells are un-transduced NK cells. In embodiments, less than 0.7% of the cells are un-transduced NK cells. In embodiments, less than 0.6% of the cells are un-transduced NK cells. In embodiments, less than 0.5% of the cells are un-transduced NK cells. In embodiments, less than 0.4% of the cells are un-transduced NK cells. In embodiments, less than 0.3% of the cells are un-transduced NK cells. In embodiments, less than 0.2% of the cells are un-transduced NK cells. In embodiments, less than 0.1% of the cells are un-transduced NK cells. In embodiments, 0% of the cells are un-transduced NK cells.

In embodiments, term “substantially pure” in reference to a particular population of Natural Killer (NK) cells as used herein, refers to a population of NK cells wherein at least 85% of the cells are of the particular type of NK cells referred to therein (e.g., NK cells including an integrated nucleic acid sequence encoding a dCas9 protein). In embodiments, at least 86% of the cells are of the particular type of NK cells referred to therein (e.g., NK cells including an integrated nucleic acid sequence encoding a dCas9 protein). In embodiments, at least 87% of the cells are of the particular type of NK cells referred to therein (e.g., NK cells including an integrated nucleic acid sequence encoding a dCas9 protein). In embodiments, at least 88% of the cells are of the particular type of NK cells referred to therein (e.g., NK cells including an integrated nucleic acid sequence encoding a dCas9 protein). In embodiments, at least 89% of the cells are of the particular type of NK cells referred to therein (e.g., NK cells including an integrated nucleic acid sequence encoding a dCas9 protein). In embodiments, at least 90% of the cells are of the particular type of NK cells referred to therein (e.g., NK cells including an integrated nucleic acid sequence encoding a dCas9 protein). In embodiments, at least 91% of the cells are of the particular type of NK cells referred to therein (e.g., NK cells including an integrated nucleic acid sequence encoding a dCas9 protein). In embodiments, at least 92% of the cells are of the particular type of NK cells referred to therein (e.g., NK cells including an integrated nucleic acid sequence encoding a dCas9 protein). In embodiments, at least 93% of the cells are of the particular type of NK cells referred to therein (e.g., NK cells including an integrated nucleic acid sequence encoding a dCas9 protein). In embodiments, at least 94% of the cells are of the particular type of NK cells referred to therein (e.g., NK cells including an integrated nucleic acid sequence encoding a dCas9 protein). In embodiments, at least 95% of the cells are of the particular type of NK cells referred to therein (e.g., NK cells including an integrated nucleic acid sequence encoding a dCas9 protein). In embodiments, at least 96% of the cells are of the particular type of NK cells referred to therein (e.g., NK cells including an integrated nucleic acid sequence encoding a dCas9 protein). In embodiments, 97% of the cells are of the particular type of NK cells referred to therein (e.g., NK cells including an integrated nucleic acid sequence encoding a dCas9 protein). In embodiments, at least 98% of the cells are of the particular type of NK cells referred to therein (e.g., NK cells including an integrated nucleic acid sequence encoding a dCas9 protein). In embodiments, at least 99% of the cells are of the particular type of NK cells referred to therein (e.g., NK cells including an integrated nucleic acid sequence encoding a dCas9 protein). In embodiments, 100% of the cells are of the particular type of NK cells referred to therein (e.g., NK cells including an integrated nucleic acid sequence encoding a dCas9 protein).

In embodiments, the population of NK cells includes less than 15% un-transduced NK cells (e.g. NK cells not including an integrated nucleic acid sequence encoding a Cas9 protein or a dCas9 protein). In embodiments, less than 14% of the cells are un-transduced NK cells. In embodiments, less than 13% of the cells are un-transduced NK cells. In embodiments, less than 12% of the cells are un-transduced NK cells. In embodiments, less than 11% of the cells are un-transduced NK cells. In embodiments, less than 10% of the cells are un-transduced NK cells. In embodiments, less than 9% of the cells are un-transduced NK cells. In embodiments, less than 8% of the cells are un-transduced NK cells. In embodiments, less than 7% of the cells are un-transduced NK cells. In embodiments, less than 6% of the cells are un-transduced NK cells. In embodiments, less than 5% of the cells are un-transduced NK cells. In embodiments, less than 4% of the cells are un-transduced NK cells. In embodiments, less than 3% of the cells are un-transduced NK cells. In embodiments, less than 2% of the cells are un-transduced NK cells. In embodiments, less than 1% of the cells are un-transduced NK cells. In embodiments, less than 0.9% of the cells are un-transduced NK cells. In embodiments, less than 0.8% of the cells are un-transduced NK cells. In embodiments, less than 0.7% of the cells are un-transduced NK cells. In embodiments, less than 0.6% of the cells are un-transduced NK cells. In embodiments, less than 0.5% of the cells are un-transduced NK cells. In embodiments, less than 0.4% of the cells are un-transduced NK cells. In embodiments, less than 0.3% of the cells are un-transduced NK cells. In embodiments, less than 0.2% of the cells are un-transduced NK cells. In embodiments, less than 0.1% of the cells are un-transduced NK cells. In embodiments, 0% of the cells are un-transduced NK cells.

In embodiments, the NK cell is obtained by transfection or transduction of an exogenous nucleic acid encoding the Cas9 protein. In embodiments, the exogenous nucleic acid encoding the Cas9 protein is present within a lentiviral vector. In embodiments, the integrated nucleic acid sequence encoding the Cas9 protein includes a lentiviral nucleic acid sequence.

In embodiments, the NK cell further includes a nucleic acid encoding an sgRNA sequence. In embodiments, the sgRNA sequence includes the nucleic acid sequence of SEQ ID NO:3, 4, 5, 6, 7, 8, 9, 10, 15, 16, 17, or 18. In embodiments, the nucleic acid encoding the sgRNA sequence is integrated into the genome of the NK cell.

In embodiments, the NK cell constitutively expresses the Cas9 protein. For example, an NK cell that constitutively expresses Cas9 protein may continuously transcribe the nucleic acid sequence encoding the Cas9 protein. Thus, in embodiments, the NK cell that constitutively expresses Cas9 continuously produces mRNA encoding the Cas9 protein. In embodiments, the NK cell that constitutively expresses Cas9 continuously produces the Cas9 protein.

In embodiments, the Cas9 protein is SpCas9, SaCas9, StCas9, NmCas9, FnCas9, CjCas9, ScCas9, SauriCas9, eSpCas9, HypaCas9, xCas9, or dCas9. In embodiments, the Cas9 protein is SpCas9. In embodiments, the Cas9 protein is SaCas9. In embodiments, the Cas9 protein is StCas9. In embodiments, the Cas9 protein is NmCas9. In embodiments, the Cas9 protein is FnCas9. In embodiments, the Cas9 protein is CjCas9. In embodiments, the ScCas9. In embodiments, the Cas9 protein is SauriCas9. In embodiments, the Cas9 protein is eSpCas9. In embodiments, the Cas9 protein is HypaCas9. In embodiments, the Cas9 protein is xCas9. In embodiments, the Cas9 protein is dCas9.

In another aspect is provided a substantially pure population of Natural Killer (NK) cells, wherein each of the NK cells includes an integrated nucleic acid sequence encoding a catalytically inactive Cas9 (dCas9) protein. In embodiments, a substantially pure population includes about 10% to 100% of NK cells provided herein including embodiments thereof.

In embodiments, the NK cells are obtained by transfection or transduction of an exogenous nucleic acid encoding said Cas9 protein. In embodiments, the transduction is a lentiviral transduction. In embodiments, each NK cell comprises a portion of the genome of the lentivirus used for transduction. In embodiments, the NK cells are further transduced with a plasmid. In embodiments, the plasmid is a sgRNA expression plasmid.

In embodiments, the dCas9 protein is constitutively expressed.

Provided herein are NK cells produced by methods provided herein including embodiments thereof. The NK cells produced by methods described herein include integrated nucleic acid sequences encoding Cas9 or variants thereof (e.g. dCas9). The NK cells may further include a nucleic acid sequence encoding a detectable protein, allowing for selection and expansion of the NK cell. In embodiments, the detectable protein is EGFP, GPF or RFP. Thus, in an aspect is provided a plurality of Natural Killer (NK) cells each including an integrated nucleic acid sequence encoding a Cas9 protein as provided herein, including embodiments thereof, wherein the plurality of NK cells is obtained by a method provided herein including embodiments thereof. In another aspect is provided a plurality of Natural Killer (NK) cells each comprising an integrated nucleic acid sequence encoding a dCas9 protein as provided herein, including embodiments thereof, wherein the plurality of NK cells is obtained by a method provided herein including embodiments thereof. METHODS OF MAKING ENGINEERED NK CELLS

Provided herein, inter alia, are methods of making the NK cells provided herein including embodiments thereof. The methods provided herein are contemplated to be useful for generating NK cells that constitutively express a Cas9 protein (e.g. dCas9, etc.). In embodiments, the methods include generating NK cells that further express a detectable protein. In embodiments, the detectable protein is used to select (e.g. by flow cytometry, etc.) NK cells from a population of cells. In embodiments, the methods provided include expanding the NK cells, thereby generating a plurality of NK cells that constitutively express the Cas9 protein. In embodiments, constitutive expression of the Cas9 protein allows for the NK cells to express proteins (e.g. IL-15, etc.) that increase NK cell cytotoxicity. In embodiments, constitutive expression of the Cas9 protein allows for NK cells to express proteins (e.g. IFN-7, etc.) that decrease NK cell exhaustion. Thus, in an aspect is provided a method of preparing a plurality of Natural Killer (NK) cells each including an integrated nucleic acid sequence encoding a Cas9 protein, the method including: (a) contacting a plurality of NK cells with a vector including a nucleic acid encoding a Cas9 protein thereby producing a transduced NK Cell, wherein the nucleic acid encoding the Cas9 protein is integrated into the genome of the NK Cell; (b) separating the transduced NK cell from the plurality of NK cells; and (c) expanding the transduced NK cell thereby producing a plurality of natural Killer (NK) cells each including an integrated nucleic acid sequence encoding the Cas9 protein.

In embodiments, the transduced NK cells expand by fold expansion, where 2 fold expansion refers to a doubling of the number of transduced NK cells. In embodiments, transduced NK cell expansion may be in the ranges of from about 2 to about 200,000, from about 20 to about 200,000, from about 100 to about 200,000, from about 1,000 to about 200,000, from about 2,000 to about 200,000, from about 5,000 to about 200,000, from about 10,000 to about 200,000, from about 20,000 to about 200,000, from about 5,000 to about 125,000, from about 15,000 to about 125,000, from about 30,000 to about 125,000, from about 45,000 to about 100,000, from about 2,000 to about 80,000, etc.

In embodiments, the cells are separated or sorted using flow cytometry or using fluorescence-activated cell sorting methods. In embodiments, the cells are separated or sorted using magnetic-activated cell sorting (MACS) methods. In embodiments, the cells are separated or sorted using a microfluidic device. In embodiments, the cells are separated or sorted using buoyancy activated cell sorting (BACS). In embodiments, the cells of interested are labeled and isolated by affinity-based column purification (e.g., affinity chromatography) sorting for the label. In embodiments, the labeled cell is isolated by affinity-based column purification sorting for the label.

In embodiments, the cells are expanded over a period of three weeks in complete RPMI media supplemented with rhIL-2 with media containing IL-2 being changed every 3 days. In embodiments, the cells are expanded over a period of about 7 days to about 28 days in complete RPMI media supplemented with rhIL-2 with media containing IL-2 being changed every 3 days. In embodiments, the cells are expanded over a period of about 10 days to about 28 days in complete RPMI media supplemented with rhIL-2 with media containing IL-2 being changed every 3 days. In embodiments, the cells are expanded over a period of about 13 days to about 28 days in complete RPMI media supplemented with rhIL-2 with media containing IL-2 being changed every 3 days. In embodiments, the cells are expanded over a period of about 16 days to about 28 days in complete RPMI media supplemented with rhIL-2 with media containing IL-2 being changed every 3 days. In embodiments, the cells are expanded over a period of about 19 days to about 28 days in complete RPMI media supplemented with rhIL-2 with media containing IL-2 being changed every 3 days. In embodiments, the cells are expanded over a period of about 22 days to about 28 days in complete RPMI media supplemented with rhIL-2 with media containing IL-2 being changed every 3 days. In embodiments, the cells are expanded over a period of about 25 days to about 28 days in complete RPMI media supplemented with rhIL-2 with media containing IL-2 being changed every 3 days. In embodiments, the cells are expanded over a period of about 28 days to about 28 days in complete RPMI media supplemented with rhIL-2 with media containing IL-2 being changed every 3 days.

In embodiments, the cells are expanded over a period of about 7 days to about 25 days in complete RPMI media supplemented with rhIL-2 with media containing IL-2 being changed every 3 days. In embodiments, the cells are expanded over a period of about 7 days to about 22 days in complete RPMI media supplemented with rhIL-2 with media containing IL-2 being changed every 3 days. In embodiments, the cells are expanded over a period of about 7 days to about 19 days in complete RPMI media supplemented with rhIL-2 with media containing IL-2 being changed every 3 days. In embodiments, the cells are expanded over a period of about 7 days to about 16 days in complete RPMI media supplemented with rhIL-2 with media containing IL-2 being changed every 3 days. In embodiments, the cells are expanded over a period of about 7 days to about 13 days in complete RPMI media supplemented with rhIL-2 with media containing IL-2 being changed every 3 days. In embodiments, the cells are expanded over a period of about 7 days to about 10 days in complete RPMI media supplemented with rhIL-2 with media containing IL-2 being changed every 3 days. In embodiments, the cells are expanded over a period of about 7 days, 10 days, 13 days, 16 days, 19 days, 22 days, 25 days, or 28 days.

In embodiments, the vector including the nucleic acid encoding the Cas9 protein is a lentiviral vector. In embodiments, the integrated nucleic acid sequence encoding a Cas9 protein includes a lentiviral nucleic acid sequence.

In embodiments, the transduced NK cells constitutively express the Cas9 protein. In embodiments, the NK cell constitutively expresses Cas9 and GFP. In embodiments, the expression of GFP is dependent on the expression of Cas9. In embodiments, the expression of GFP is used as a indicator of the expression of Cas9.

In embodiments, the transduced NK cells further include a nucleic acid encoding an sgRNA sequence. In embodiments, the sgRNA sequence includes the nucleic acid sequence of SEQ ID NO:3, 4, 5, 6, 7, 8, 9, 10, 15, 16, 17, or 18. In embodiments, the nucleic acid encoding the sgRNA sequence integrated into the genome of the NK cells.

In embodiments, the method further includes measuring Cas9 activity of the transduced NK cells prior to the expanding. Methods for measuring Cas9 activity include measuring expression levels of a target gene. Any method known in the art for measuring gene expression may be used, including but not limited to qPCR, microarray, Northern blot, Western blot, and enzyme-linked immunosorbent assay (ELISA). For example, expression levels of a target gene (e.g. IL-15, INF-7, a type I interferon, etc.) may be determined by measuring mRNA levels using qPCR methods.

Provided herein, inter alia, are methods for making dCas9-expressing NK cells. In embodiments, the methods provided herein are effective for generating NK cells that constitutively express dCas9. Thus, in another aspect is provided a method of preparing a plurality of Natural Killer (NK) cells each including an integrated nucleic acid sequence encoding a catalytically inactive Cas9 (dCas9) protein, the method including. (a) contacting a plurality of NK cells with a vector including a nucleic acid encoding a dCas9 protein, thereby producing a transduced NK Cell wherein the nucleic acid encoding the dCas9 protein is integrated into the genome of the NK Cell; (b) separating the transduced NK cell from the plurality of NK cells; and (c) expanding the transduced NK cell thereby producing a plurality of natural Killer (NK) cells each including an integrated nucleic acid sequence encoding the dCas9 protein.

In embodiments, the vector including the nucleic acid sequence encoding dCas9 is capable of integrating the nucleic acid into the host genome. In embodiments, the vector including the nucleic acid encoding the catalytically inactive Cas9 (dCas9) protein is a lentiviral vector. In embodiments, the integrated nucleic acid sequence encoding a catalytically inactive Cas9 (dCas9) protein includes a lentiviral nucleic acid sequence. In embodiments, the transduced NK cells constitutively express the dCas9 protein.

In embodiments, the transduced NK cells further include a nucleic acid encoding an sgRNA sequence. In embodiments, the sgRNA sequence includes the nucleic acid sequence of SEQ ID NO:3, 4, 5, 6, 7, 8, 9, 10, 15, 16, 17, or 18. In embodiments, the nucleic acid encoding the sgRNA sequence integrated into the genome of the NK cells.

For the methods provided herein, in embodiments, the dCas9 protein is fused to a transcriptional activator or a transcriptional inactivator. The transcriptional activator may, for example, increase or upregulate expression of one or more genes (e.g. INF-α, INF-β, INF-γ, IL-15, etc.) in the NK cell. The transcriptional inactivator may, for example, inhibit or downregulate expression of one or more genes that cause suppress NK cell cytotoxicity. Thus, in embodiments, the dCas9 protein is fused to a transcriptional activator. In embodiments, the dCas9 protein is fused to a transcriptional inactivator.

In embodiments, the transcriptional activator is VP64. In embodiments, the transcriptional activator is VP16. In embodiments, the transcriptional activator is VP48. In embodiments, the transcriptional activator is VP64. In embodiments, VP64 includes the sequence of SEQ ID NO:25. In embodiments, VP64 has at least 80% sequence identity to SEQ ID NO:25. In embodiments, VP64 has at least 85% sequence identity to SEQ ID NO:25. In embodiments, VP64 has at least 90% sequence identity to SEQ ID NO:25. In embodiments, VP64 has at least 95% sequence identity to SEQ ID NO:25.

In embodiments, the transcriptional activator is the transactivator domain of nuclear factor kappa B (p65). In embodiments, the transactivator domain of p65 includes the sequence of SEQ ID NO:23. In embodiments, the transactivator domain of p65 has at least 80% sequence identity to SEQ ID NO:23. In embodiments, the transactivator domain of p65 has at least 85% sequence identity to SEQ ID NO:23. In embodiments, the transactivator domain of p65 has at least 90% sequence identity to SEQ ID NO:23. In embodiments, the transactivator domain of p65 has at least 95% sequence identity to SEQ ID NO:23. In embodiments, the transactivator domain of p65 has at least 98% sequence identity to SEQ ID NO:23. In embodiments, the transactivator domain of p65 includes the sequence of SEQ ID NO:26. In embodiments, the transactivator domain of p65 has at least 80% sequence identity to SEQ ID NO:26. In embodiments, the transactivator domain of p65 has at least 85% sequence identity to SEQ ID NO:26. In embodiments, the transactivator domain of p65 has at least 90% sequence identity to SEQ ID NO:26. In embodiments, the transactivator domain of p65 has at least 95% sequence identity to SEQ ID NO:26. In embodiments, the transactivator domain of p65 has at least 98% sequence identity to SEQ ID NO:26.

In embodiments, the transcriptional activator is the Epstein-Barr virus R transactivator (Rta). In embodiments, Rta includes the sequence of SEQ ID NO:24. In embodiments, Rta has at least 80% sequence identity to SEQ ID NO:24. In embodiments, Rta has at least 85% sequence identity to SEQ ID NO:24. In embodiments, Rta has at least 90% sequence identity to SEQ ID NO:24. In embodiments, Rta has at least 95% sequence identity to SEQ ID NO:24. In embodiments, Rta has at least 98% sequence identity to SEQ ID NO:24. In embodiments, the transcriptional activator is VP160. In embodiments, the transcriptional activator is VP64-p65-Rta (VPR), in which the p65 and Rta transcriptional activators are fused to the C-terminus end of VP64.

In embodiments, the dCas9 construct is SunTag (dcas9-GCN4/sgRNA plus scFV-VP64), wherein the dCas9 is fused to a multimeric peptide array (SunTag) that each peptide binds to one single-chain variable fragment (scFv) that is fused to VP64. In embodiments, the dCas9 construct is the synergistic activation mediator (SAM) system which combines dCas9-VP64 with a modified sgRNA containing two MS2 hairpins, in which each MS2 hairpin interacts with one MS2 binding protein (MCP) that is fused to the transactivator domain of p65 and the human heat shock factor 1 (HSF1). In embodiments, the dCas9 construct is is SPH (dCas9-GCN4/sgRNA plus scFV-p65-HSF1), wherein the SAM is combined with SunTag, or using the SunTag system to recruit p65AD-HSF1 instead of VP64 (SPH).

In embodiments, the transcriptional inactivator is KRAB (Krüppel associated box) domain of Kox1. In embodiments, KRAB includes the sequence of SEQ ID NO:21. In embodiments, KRAB has at least 80% sequence identity to SEQ ID NO:21. In embodiments, KRAB has at least 85% sequence identity to SEQ ID NO:21. In embodiments, KRAB has at least 90% sequence identity to SEQ ID NO:21. In embodiments, KRAB has at least 95% sequence identity to SEQ ID NO:21. In embodiments, KRAB has at least 98% sequence identity to SEQ ID NO:21. In embodiments, the transcriptional inactivator is the WRPW domain of Hes1. In embodiments, the transcriptional inactivator is the CS (Chromo Shadow) domain of HP1α. In embodiments, the transcriptional inactivator is the SID4X domain.

In embodiments, the method further includes measuring the activity of the dCas9 protein fused to VP64 (dCas9-VP64) prior to the expanding. Measuring the activity of the dCas9 includes any method known in the art for measuring expression of a target gene, including but not limited to qPCR, microarray, Northern blot, Western blot, and enzyme-linked immunosorbent assay (ELISA). In embodiments, the method further includes measuring the activity of the dCas9 protein fused to VP64 (dCas9-VP64) by qPCR prior to the expanding. For example, mRNA levels of the target gene may be measured by qPCR. In embodiments, the activity of a protein (e.g. dCas9-VP64) is an increase of expression of a target gene relative to the expression of the target gene in the absence of a transcriptional activator (e.g. VP64, etc.) as disclosed herein. In embodiments, the activity of a protein (e.g. dCas9-VP64) is an increase of expression of a target gene relative to the expression of the target gene in the absence of the protein as disclosed herein.

In embodiments, the expression of the target gene is increased by at least 10%. In embodiments, the expression of the target gene is increased by at least 20%. In embodiments, the expression of the target gene is increased by at least 30%. In embodiments, the expression of the target gene is increased by at least 40%. In embodiments, the expression of the target gene is increased by at least 50%. In embodiments, the expression of the target gene is increased by at least 60%. In embodiments, the expression of the target gene is increased by at least 70%. In embodiments, the expression of the target gene is increased by at least 80%. In embodiments, the expression of the target gene is increased by at least 90%. In embodiments, the expression of the target gene is increased by at least 95%. In embodiments, the expression of the target gene is increased by at least 100%.

In embodiments, the expression of the target gene is increased by at least 2-fold. In embodiments, the expression of the target gene is increased by at least 3-fold. In embodiments, the expression of the target gene is increased by at least 4-fold. In embodiments, the expression of the target gene is increased by at least 5-fold. In embodiments, the expression of the target gene is increased by at least 6-fold. In embodiments, the expression of the target gene is increased by at least 7-fold. In embodiments, the expression of the target gene is increased by at least 8-fold. In embodiments, the expression of the target gene is increased by at least 9-fold. In embodiments, the expression of the target gene is increased by at least 10-fold. In embodiments, the expression of the target gene is increased by at least 20-fold. In embodiments, the expression of the target gene is increased by at least 30-fold. In embodiments, the expression of the target gene is increased by at least 40-fold. In embodiments, the expression of the target gene is increased by at least 50-fold. In embodiments, the expression of the target gene is increased by at least 60-fold. In embodiments, the expression of the target gene is increased by at least 70-fold. In embodiments, the expression of the target gene is increased by at least 80-fold. In embodiments, the expression of the target gene is increased by at least 90-fold. In embodiments, the expression of the target gene is increased by at least 100-fold.

In embodiments, the activity of a protein (e.g. dCas9-KRAB) is an decrease of expression of a target gene relative to the expression of the target gene in the absence of a transcriptional inactivator (e.g. KRAB, etc.) as disclosed herein. In embodiments, the activity of a protein (e.g. dCas9-KRAB) is an increase of expression of a target gene relative to the expression of the target gene in the absence of the protein as disclosed herein.

In embodiments, the expression of the target gene is decreased by at least 10%. In embodiments, the expression of the target gene is decreased by at least 20%. In embodiments, the expression of the target gene is decreased by at least 30%. In embodiments, the expression of the target gene is decreased by at least 40%. In embodiments, the expression of the target gene is decreased by at least 50%. In embodiments, the expression of the target gene is decreased by at least 10%. In embodiments, the expression of the target gene is decreased by at least 60%. In embodiments, the expression of the target gene is decreased by at least 70%. In embodiments, the expression of the target gene is decreased by at least 80%. In embodiments, the expression of the target gene is decreased by at least 90%. In embodiments, the expression of the target gene is decreased by at least 95%. In embodiments, the expression of the target gene is decreased by at least 97%. In embodiments, the expression of the target gene is decreased by at least 98%. In embodiments, the expression of the target gene is decreased by at least 99%. In embodiments, the expression of the target gene is decreased by at least 99.5%. In embodiments, the expression of the target gene is decreased by at least 99.9%.

In embodiments, the expression of the target gene is decreased by at least 2-fold. In embodiments, the expression of the target gene is decreased by at least 3-fold. In embodiments, the expression of the target gene is decreased by at least 4-fold. In embodiments, the expression of the target gene is decreased by at least 5-fold. In embodiments, the expression of the target gene is decreased by at least 6-fold. In embodiments, the expression of the target gene is decreased by at least 7-fold. In embodiments, the expression of the target gene is decreased by at least 8-fold. In embodiments, the expression of the target gene is decreased by at least 9-fold. In embodiments, the expression of the target gene is decreased by at least 10-fold. In embodiments, the expression of the target gene is decreased by at least 20-fold. In embodiments, the expression of the target gene is decreased by at least 30-fold. In embodiments, the expression of the target gene is decreased by at least 40-fold. In embodiments, the expression of the target gene is decreased by at least 50-fold.

### Methods of Modifying Gene Expression

Methods provided herein including embodiments thereof are contemplated to be effective for modifying gene expression in a subject. For example, the subject may be administered an NK cell expressing dCas fused to a transcriptional activator (e.g. dCAS9-VP64) or transcriptional inactivator (dCAS9-KRAB). Administration of the NK cells provided herein to a subject may modify expression levels of genes associated with disease pathways, for example, genes that are repressed by the MYC oncogene.

Thus, in an aspect is provided a method of increasing expression of a target gene within a subject, the method including administering to the subject a plurality of Natural Killer (NK) cells, wherein each of the plurality NK cell includes an integrated nucleic acid sequence encoding a catalytically inactive Cas9 (dCas9). In embodiments, an effective amount of NK cells are administered to the subject. In embodiments, the dCas9 protein is fused to a transcriptional activator. In embodiments, the transcriptional activator is VP64.

In another aspect is provided a method of decreasing expression of a target gene within a subject, the method including administering to the subject a plurality of Natural Killer (NK) cells, wherein each of the plurality NK cell includes an integrated nucleic acid sequence encoding a catalytically inactive Cas9 (dCas9). In embodiments, an effective amount of NK cells are administered to the subject. In embodiments, the dCas9 protein is fused to a transcriptional inactivator. In embodiments, the transcriptional inactivator is KRAB.

For the methods provided herein, in embodiments, the subject has been diagnosed with a cancer or is being treated for a cancer. In embodiments, the cancer is leukemia. In embodiments, the leukemia is B- and T-cell acute lymphoblastic leukemia (B/T-ALL). In embodiments, the leukemia is acute myloid leukemia (AML).

In embodiments, the plurality of NK cells are allogeneic NK cells. In embodiments, the plurality of NK cells are autologous NK Cells.

In embodiments, the target gene is IL-15, IFNα2, IFNα1, IFNβ1, STAT1, STAT2, IFNAR 1, IFNAR 2, NKG2D, NKp46, DNAM-1 or CD96. In embodiments, the target gene is IL-15. In embodiments, the target gene is IFNα2. In embodiments, the target gene is IFNα1. In embodiments, the target gene is IFNβ1. In embodiments, the target gene is STAT1. In embodiments, the target gene is STAT2. In embodiments, the target gene is IFNAR 1. In embodiments, the target gene is IFNAR 2. In embodiments, the target gene is NKG2D. In embodiments, the target gene is NKp46. In embodiments, the target gene is DNAM-1. In embodiments, the target gene is CD96.

Provided herein are methods for decreasing expression of a target gene in a subject. The methods may be effective for decreasing a expression of a gene associated with a disease (e.g. AML). Thus, in another aspect is provided a method of decreasing the expression of a target gene within a subject, the method including administering to the subject a plurality of NK cells, wherein each of the plurality NK cells includes an integrated nucleic acid sequence encoding a Cas9. In embodiments, the Cas9 is a catalytically inactive Cas9 (dCas9).

In embodiments, the subject has been diagnosed with a cancer or is being treated for a cancer. Thus, in embodiments, a method of treating cancer in a subject in need thereof is provided. In embodiments, the cancer is leukemia. In embodiments, the leukemia is B- and T-cell acute lymphoblastic leukemia (B/T-ALL). In embodiments, the leukemia is acute myeloid leukemia (AML).

In embodiments, the method is for use in an allogeneic cell therapy or an autologous cell therapy. In embodiments, the method is for use in an allogeneic cell therapy. In embodiments, the method is for use in an autologous cell therapy.

In embodiments, the target gene is STAT1, STAT2, IFNAR 1, IFNAR 2, NKG2D, NKp46, PD1, DNAM-1, PVRIG or CD96. In embodiments, the target gene is STAT1. In embodiments, the target gene is STAT2. In embodiments, the target gene is IFNAR 1. In embodiments, the target gene is IFNAR 2. In embodiments, the target gene is NKG2D. In embodiments, the target gene is NKp46. In embodiments, the target gene is PD1. In embodiments, the target gene is DNAM-1. In embodiments, the target gene is PVRIG. In embodiments, the target gene is CD96.

### Methods of Treatment

The NK cells provided herein including embodiments thereof are contemplated to be effective for the treatment of diseases, and in particular cancer (e.g. leukemia). For example, the NK cells may be effective in modulating gene expression in a subject, wherein the modulated gene expression increases the cytotoxic effect of immune cells in response to the cancer. Thus, in an aspect is provided a method of treating or preventing a disease in a subject in need thereof, the method including administering to the subject a therapeutically effective amount of an NK cell capable of expressing an elevated level of a type I interferon relative to a standard control NK cell, or an elevated level of IL-15 relative to the standard control NK cell. In embodiments, the standard control NK cell is a standard NK cell line such as NK-92 or NK-101. In embodiments, the standard NK cell line is NK-92 cell line. In embodiments, the standard NK line is NK-101. In embodiments, the standard control NK cell does not include a nucleic acid sequence encoding a Cas9 (e.g. dCas9) protein. In embodiments, the standard control NK cell does not include an integrated nucleic acid sequence encoding a Cas9 (e.g. dCas9) protein. In embodiments, the standard control NK cell does not constitutively express a Cas9 (e.g. dCas9) protein.

In embodiments, the expression of the type I interferon in the NK cell is increased by at least 10% relative to the control NK cell. In embodiments, the expression of the type I interferon in the NK cell is increased by at least 20% relative to the control NK cell. In embodiments, the expression of the type I interferon in the NK cell is increased by at least 30% relative to the control NK cell. In embodiments, the expression of the type I interferon in the NK cell is increased by at least 40% relative to the control NK cell. In embodiments, the expression of the type I interferon in the NK cell is increased by at least 50% relative to the control NK cell. In embodiments, the expression of the type I interferon in the NK cell is increased by at least 60% relative to the control NK cell. In embodiments, the expression of the type I interferon in the NK cell is increased by at least 70% relative to the control NK cell. In embodiments, the expression of the type I interferon in the NK cell is increased by at least 80% relative to the control NK cell. In embodiments, the expression of the type I interferon in the NK cell is increased by at least 90% relative to the control NK cell. In embodiments, the expression of the type I interferon in the NK cell is increased by at least 100% relative to the control NK cell.

In embodiments, the expression of the type I interferon in the NK cell is increased by at least 1-fold relative to the control NK cell. In embodiments, the expression of the type I interferon in the NK cell is increased by at least 2-fold relative to the control NK cell. In embodiments, the expression of the type I interferon in the NK cell is increased by at least 3-fold relative to the control NK cell. In embodiments, the expression of the type I interferon in the NK cell is increased by at least 4-fold relative to the control NK cell. In embodiments, the expression of the type I interferon in the NK cell is increased by at least 5-fold relative to the control NK cell. In embodiments, the expression of the type I interferon in the NK cell is increased by at least 10-fold relative to the control NK cell. In embodiments, the expression of the type I interferon in the NK cell is increased by at least 15-fold relative to the control NK cell. In embodiments, the expression of the type I interferon in the NK cell is increased by at least 20-fold relative to the control NK cell. In embodiments, the expression of the type I interferon in the NK cell is increased by at least 50-fold relative to the control NK cell. In embodiments, the expression of the type I interferon in the NK cell is increased by at least 100-fold relative to the control NK cell.

In embodiments, the expression of IL-15 in the NK cell is increased by at least 10% relative to the control NK cell. In embodiments, the expression of IL-15 in the NK cell is increased by at least 20% relative to the control NK cell. In embodiments, the expression of IL-15 in the NK cell is increased by at least 30% relative to the control NK cell. In embodiments, the expression of IL-15 in the NK cell is increased by at least 40% relative to the control NK cell. In embodiments, the expression of IL-15 in the NK cell is increased by at least 50% relative to the control NK cell. In embodiments, the expression of IL-15 in the NK cell is increased by at least 60% relative to the control NK cell. In embodiments, the expression of IL-15 in the NK cell is increased by at least 70% relative to the control NK cell. In embodiments, the expression of IL-15 in the NK cell is increased by at least 80% relative to the control NK cell. In embodiments, the expression of IL-15 in the NK cell is increased by at least 90% relative to the control NK cell. In embodiments, the expression of IL-15 in the NK cell is increased by at least 100% relative to the control NK cell.

In embodiments, the expression of IL-15 in the NK cell is increased by at least 1-fold relative to the control NK cell. In embodiments, the expression of IL-15 in the NK cell is increased by at least 2-fold relative to the control NK cell. In embodiments, the expression of IL-15 in the NK cell is increased by at least 3-fold relative to the control NK cell. In embodiments, the expression of IL-15 in the NK cell is increased by at least 4-fold relative to the control NK cell. In embodiments, the expression of IL-15 in the NK cell is increased by at least 5-fold relative to the control NK cell. In embodiments, the expression of IL-15 in the NK cell is increased by at least 10-fold relative to the control NK cell. In embodiments, the expression of IL-15 in the NK cell is increased by at least 15-fold relative to the control NK cell. In embodiments, the expression of IL-15 in the NK cell is increased by at least 20-fold relative to the control NK cell. In embodiments, the expression of IL-15 in the NK cell is increased by at least 50-fold relative to the control NK cell. In embodiments, the expression of IL-15 in the NK cell is increased by at least 100-fold relative to the control NK cell.

In embodiments, the NK cell provided herein (e.g., NK cells including an integrated nucleic acid sequence encoding a dCas9 protein) has substantially similar cytotoxicity compared to a control NK cell. In embodiments, an NK cell provided herein expresses substantially similar levels of CD69 compared to a control NK cell. For example, the NK cell provided herein may express 70%, 75%, 80%, 85%, 90% 95% or 100% levels of CD69 as compared to a control NK cell. In embodiments, the NK cell provided herein expresses at least 70% CD69 levels as compared to a control NK cell. In embodiments, the NK cell provided herein expresses at least 75% CD69 levels as compared to a control NK cell. In embodiments, the NK cell provided herein expresses at least 80% CD69 levels as compared to a control NK cell. In embodiments, the NK cell provided herein expresses at least 85% CD69 levels as compared to a control NK cell. In embodiments, the NK cell provided herein expresses at least 90% CD69 levels as compared to a control NK cell. In embodiments, the NK cell provided herein expresses at least 95% CD69 levels as compared to a control NK cell. In embodiments, the NK cell provided herein expresses 100% of CD69 as compared to a control NK cell. In embodiments, cytotoxicity is measured as cancer cell death. Cancer cell death may be measured in a variety of methods well known in the art including but not limited to measuring annexin V binding, flow cytometry, caspase activation and detection, mitochondrial membrane potential-dependent cyes and cytochrome C release assays. Thus, in embodiments, the NK cell provided herein induce at least 60%, 70%, 75%, 80%, 85%, 90% 95% or 100% cell death as compared to a control NK cell. In embodiments, the NK cell provided herein induces at least 60% cell death as compared to a control NK cell. The NK cell provided herein induces at least 70% cell death as compared to a control NK cell. In embodiments, the NK cell provided herein induces at least 75% cell death as compared to a control NK cell. In embodiments, the NK cell provided herein induces at least 80% cell death as compared to a control NK cell. In embodiments, the NK cell provided herein induces at least 85% cell death as compared to a control NK cell. In embodiments, the NK cell provided herein induces at least 90% cell death as compared to a control NK cell. In embodiments, the NK cell provided herein induces at least 95% cell death as compared to a control NK cell. In embodiments, the NK cell provided herein induces 100% cell death as compared to a control NK cell.

In embodiments, the NK cell provided herein (e.g., NK cells including an integrated nucleic acid sequence encoding a dCas9 protein) has similar levels of proliferation as compared to a control NK cell. For example, cell cultures including the NK cell provided herein or a control NK cell may produce substantially the same number of NK cells. For example, an NK cell provided herein may proliferate at at least 70%, 75%, 80%, 85%, 90%, 95%, 95%, or 100% of the levels as a control NK cell.

In embodiments, the NK cell is tissue-matched to the subject. The term “tissue matched” is used in accordance to its ordinary meaning in the art and refers to matching HLA type between a donor and a recipient of a transplant (e.g. a cell (e.g. NK cell), tissue or organ). In embodiments, the NK cell is derived from a healthy subject and wherein the standard control NK cell is an NK cell obtained from the healthy subject. In embodiments, the healthy subject does not have cancer. In embodiments, the healthy subject does not have leukemia. In embodiments, the healthy subject does not have acute lymphoblastic leukemia.

In embodiments, the subject in need thereof has cancer. In embodiments, the subject has leukemia. In embodiments, the subject has acute lymphoblastic leukemia.

In embodiments, the NK cell includes an integrated nucleic acid sequence encoding a Cas9 protein. In embodiments, the Cas9 protein is a catalytically inactive Cas9 (dCas9) protein. In embodiments, the nucleic acid encoding the Cas9 protein further encodes a transcriptional activator thereby encoding a site-specific modifying polypeptide-transcriptional activator fusion protein. In embodiments, the transcriptional activator is VP64. In embodiments, the Cas9-transcriptional activator fusion protein is a dCas9-VP64 fusion protein. In embodiments, the NK cell further includes a nucleic acid encoding an sgRNA. In embodiments, the sgRNA sequence includes the nucleic acid sequence of SEQ ID NO:3, 4, 5, 6, 7, 8, 9, 10, 15, 16, 17, or 18.

In embodiments, the sgRNA targets a type I interferon gene or an IL-15 gene. In embodiments, the sgRNA targets a type I interferon gene and an IL-15 gene. In embodiments, the type I interferon is an IFN-α gene or an IFN-β gene. In embodiments, the type I interferon is an IFN-α gene. In embodiments, the type I interferon is an IFN-β gene. In embodiments, the IFN-α gene is an IFN-α1 gene or an IFN-α2 gene. In embodiments, the IFN-α gene is an IFN-α1 gene. In embodiments, the IFN-α gene is IFN-α2 gene. In embodiments, the IFN-β gene is an IFN-β1 gene.

In embodiments, the NK cell is capable of expressing an elevated level of a type I interferon relative to a standard control NK cell and an elevated level of IL-15 relative to a standard control NK cell. In embodiments, the type I interferon is an IFN-α gene or an IFN-β gene. In embodiments, the type I interferon is an IFN-α gene. In embodiments, the type I interferon is an IFN-β gene. In embodiments, the type I interferon is an IFN-α1 gene, an IFN-α2 gene or an IFN-β1. In embodiments, the IFN-α gene is an IFN-α1 gene. In embodiments, the IFN-α gene is IFN-α2 gene. In embodiments, the IFN-β gene is an IFN-β1 gene.

In an aspect, provided is a method of treating leukemia in a subject in need thereof, the method including administering to said subject an effective amount of the NK cell provided herein including embodiments thereof. In embodiments, the leukemia is acute lymphoblastic leukemia.

In an aspect is provided a method of preventing recurrence of leukemia in a subject, the method including administering to said subject an effective amount of an natural killer (NK) cell capable of expressing an elevated level of a type I interferon relative to a standard control NK cell or an elevated level of IL-15 relative to the standard control NK cell. “Recurrence” is used in accordance to its ordinary meaning in the art and refers to when a cancer (e.g. leukemia) returns after an initial decrease or disappearance of symptoms of the cancer, or after cancer cells are no longer detectable in the subject. In embodiments, the subject was previously treated for leukemia. In embodiments, the subject was not responsive to the previous treatment. In embodiments, the subject was responsive to the previous treatment and is no longer responsive to the treatment. In embodiments, the leukemia is acute lymphoblastic leukemia.

The NK cells provided herein including embodiments thereof are contemplated to be effective for inducing an immune response in a subject in need thereof. For example, the NK cells provided herein may have prophylactic activity such that the NK cells can prevent or reduce a likelihood of the occurrence of a disease or condition in a subject. Thus, in another aspect is provided a method of treating or preventing an infectious disease in a leukemia patient, the method including administering an effective amount of an NK cell provided herein including embodiments thereof. In embodiments, the leukemia is acute lymphoblastic leukemia. In embodiments, the infectious disease is a viral infection. In embodiments, the infectious disease is an influenza infection. In embodiments, the method further includes treating leukemia in the subject.

### EMBODIMENTS

Embodiment 1. A Natural Killer (NK) cell comprising an integrated nucleic acid sequence encoding a catalytically inactive Cas9 (dCas9) protein.

Embodiment 2. The NK cell of embodiment 1, wherein said NK cell is obtained by transduction of an exogenous nucleic acid encoding said catalytically inactive Cas9 (dCas9) protein.

Embodiment 3. The NK cell of embodiment 2, wherein the exogenous nucleic acid encoding said catalytically inactive Cas9 (dCas9) protein is present within a lentiviral vector.

Embodiment 4. The NK cell of embodiment 3, wherein said integrated nucleic acid sequence encoding a catalytically inactive Cas9 (dCas9) protein comprises a lentiviral nucleic acid sequence.

Embodiment 5. The NK cell of any one of embodiments 1-4, wherein said NK cell further comprises a nucleic acid encoding an sgRNA sequence.

Embodiment 6. The NK cell of embodiment 5, wherein the nucleic acid encoding said sgRNA sequence is integrated into the genome of said NK cell.

Embodiment 7. The NK cell of any one of embodiments 1-6, wherein the NK cell constitutively expresses said dCas9 protein.

Embodiment 8. A substantially pure population of Natural Killer (NK) cells, wherein each of said NK cells comprises an integrated nucleic acid sequence encoding a Cas9 protein.

Embodiment 9. The NK cell of embodiment 8, wherein said NK is obtained by transduction of an exogenous nucleic acid encoding said Cas9 protein.

Embodiment 10. The NK cell of embodiment 9, wherein the exogenous nucleic acid encoding said Cas9 protein is present within a lentiviral vector.

Embodiment 11. The NK cell of any one of embodiments 8-10, wherein said integrated nucleic acid sequence encoding a Cas9 protein comprises a lentiviral nucleic acid sequence.

Embodiment 12. The NK cell of any one of embodiments 8-11, wherein said NK cell further comprises a nucleic acid encoding an sgRNA sequence.

Embodiment 13. The NK cell of embodiment 12, wherein the nucleic acid encoding said sgRNA sequence is integrated into the genome of said NK cell.

Embodiment 14. The NK cell of of any one of embodiments 8-13, wherein the NK cell constitutively expresses said Cas9 protein.

Embodiment 15. A substantially pure population of Natural Killer (NK) cells, wherein each of said NK cells comprises an integrated nucleic acid sequence encoding a catalytically inactive Cas9 (dCas9) protein.

Embodiment 16. The substantially pure population of NK cells of embodiment 15, wherein said NK cells are obtained by transduction.

Embodiment 17. The substantially pure population of NK cells of embodiment 16, wherein the transduction is a lentiviral transduction.

Embodiment 18. The substantially pure population of NK cells of embodiment 17, wherein each NK cell comprises a portion of the genome of the lentivirus used for transduction.

Embodiment 19. The substantially pure population of NK cells of any one of embodiments 16-18, wherein said NK cells are further transduced with a plasmid.

Embodiment 20. The substantially pure population of NK cells of embodiment 19, wherein the plasmid is a sgRNA expression plasmid.

Embodiment 21. The substantially pure population of NK cells of any one of embodiments 15-20, wherein the dCas9 protein is constitutively expressed.

Embodiment 22. A method of preparing a plurality of Natural Killer (NK) cells each comprising an integrated nucleic acid sequence encoding a Cas9 protein, the method comprising: (a) contacting a plurality of NK cells with a vector comprising a nucleic acid encoding a Cas9 protein thereby producing a transduced NK Cell wherein said nucleic acid encoding said Cas9 protein is integrated into the genome of said NK Cell; (b) separating said transduced NK cell from said plurality of NK cells; and expanding said transduced NK cell thereby producing a plurality of natural Killer (NK) cells each comprising an integrated nucleic acid sequence encoding said Cas9 protein.

Embodiment 23. The method of embodiment 22, wherein the vector comprising the nucleic acid encoding said Cas9 protein is a lentiviral vector.

Embodiment 24. The NK cell of embodiment 22 or 23, wherein said integrated nucleic acid sequence encoding a Cas9 protein comprises a lentiviral nucleic acid sequence.

Embodiment 25. The method of any one of embodiments 22-24, wherein the transduced NK cells constitutively express said Cas9 protein.

Embodiment 26. The method of any one of embodiments 22-25, wherein the transduced NK cells further comprise a nucleic acid encoding an sgRNA sequence.

Embodiment 27. The method of embodiment 26, wherein the nucleic acid encoding said sgRNA sequence integrated into the genome of said NK cells.

Embodiment 28. The method of embodiments 22-27, further comprising measuring Cas9 activity of the transduced NK cells prior to said expanding.

Embodiment 29. A plurality of Natural Killer (NK) cells each comprising an integrated nucleic acid sequence encoding a Cas9 protein obtained by the method of any one of embodiments 22-28.

Embodiment 30. A method of preparing a plurality of Natural Killer (NK) cells each comprising an integrated nucleic acid sequence encoding a catalytically inactive Cas9 (dCas9) protein, the method comprising: (a) contacting a plurality of NK cells with a vector comprising a nucleic acid encoding a dCas9 protein thereby producing a transduced NK Cell wherein said nucleic acid encoding said dCas9 protein is integrated into the genome of said NK Cell; (b) separating said transduced NK cell from said plurality of NK cells; and expanding said transduced NK cell thereby producing a plurality of natural Killer (NK) cells each comprising an integrated nucleic acid sequence encoding said dCas9 protein.

Embodiment 31. The method of embodiment 30, wherein the vector comprising the nucleic acid encoding said catalytically inactive Cas9 (dCas9) protein is a lentiviral vector.

Embodiment 32. The NK cell of embodiment 31, wherein said integrated nucleic acid sequence encoding a catalytically inactive Cas9 (dCas9) protein comprises a lentiviral nucleic acid sequence.

Embodiment 33. The method of any one of embodiments 30-32, wherein the transduced NK cells constitutively express said dCas9 protein.

Embodiment 34. The method of any one of embodiments 30-33, wherein the transduced NK cells further comprise a nucleic acid encoding an sgRNA sequence.

Embodiment 35. The method of embodiment 34, wherein the nucleic acid encoding said sgRNA sequence integrated into the genome of said NK cells.

Embodiment 36. The method of any one of embodiments 30-35, wherein the dCas9 protein is fused to a transcriptional activator.

Embodiment 37. The method of any one of embodiments 30-35, wherein the dCas9 protein is fused to a transcriptional inactivator.

Embodiment 38. The method of embodiment 36, wherein the transcriptional activator is VP64.

Embodiment 39. The method of embodiment 38, further comprising measuring the activity of the dCas9 protein fused to VP64 (dCas9-VP64) by qPCR prior to said expanding.

Embodiment 40. The method of embodiment 39, wherein the activity is an increase of expression of a target gene.

Embodiment 41. The method of embodiment 40, wherein the expression of the target gene is increased by at least 2-fold.

Embodiment 42. A plurality of Natural Killer (NK) cells each comprising an integrated nucleic acid sequence encoding a dCas9 protein obtained by the method of any one of embodiments 30-41.

Embodiment 43. A method of increasing expression of a target gene within a subject, the method comprising administering to the subject a plurality of Natural Killer (NK) cells, wherein each of said plurality NK cell comprises an integrated nucleic acid sequence encoding a catalytically inactive Cas9 (dCas9).

Embodiment 44. The method of embodiment 43, wherein the dCas9 protein is fused to a transcriptional activator.

Embodiment 45. The method of embodiment 44, wherein the transcriptional activator is VP64.

Embodiment 46. The method of any one of embodiments 43-45, wherein the subject has been diagnosed with a cancer or is being treated for a cancer.

Embodiment 47. The method of any one of embodiments 43-46, wherein the plurality of NK cells are allogeneic NK cells.

Embodiment 48. The method of any one of embodiments 43-47, wherein the plurality of NK cells are autologous NK Cells.

Embodiment 49. The method of any one of embodiments 43-48, wherein the target gene is selected from the group consisting of IL-15, IFNα2, IFNα1, IFNβ1, STAT1, STAT2, IFNAR 1, IFNAR 2, NKG2D, NKp46, DNAM-1 and CD96.

Embodiment 50. A method of decreasing the expression of a target gene within a subject, the method comprising administering to the subject a plurality of NK cells, wherein each of said plurality NK cells comprises an integrated nucleic acid sequence encoding a Cas9 protein.

Embodiment 51. The method of embodiment 50, wherein the subject has been diagnosed with a cancer or is being treated for a cancer.

Embodiment 52. The method of embodiment 50 or 51, for use in an allogeneic cell therapy.

Embodiment 53. The method of any one of embodiments 50-52, wherein the target gene is selected from the group consisting of STAT1, STAT2, IFNAR 1, IFNAR 2, NKG2D, NKp46, PD1, DNAM-1, PVRIG and CD96.

Embodiment 54. A method of treating or preventing a disease in a subject in need thereof, the method comprising administering to said subject a therapeutically effective amount of an NK cell capable of expressing an elevated level of a type I interferon or an elevated level of IL-15 relative to a standard control NK cell.

Embodiment 55. The method of embodiment 54, wherein said NK cell is tissue-matched to the subject.

Embodiment 56. The method of embodiment 54 or 55, wherein the disease is cancer.

Embodiment 57. The method of any one of embodiments 54-56, wherein the disease is leukemia.

Embodiment 58. The method any one of embodiments 54-57, wherein the disease is acute lymphoblastic leukemia.

Embodiment 59. The method any one of embodiments 54 to 58, wherein said NK cell comprises an integrated nucleic acid sequence encoding a Cas9 protein.

Embodiment 60. The method of embodiment 59 wherein the Cas9 protein is a catalytically inactive Cas9 (dCas9) protein.

Embodiment 61. The method of one of embodiments 54 to 60 wherein said nucleic acid encoding said Cas9 protein further encodes a transcriptional activator thereby encoding a Cas9 protein-transcriptional activator fusion protein.

Embodiment 62. The method of embodiment 61, wherein said transcriptional activator is VP64.

Embodiment 63. The method of one of embodiment 61, wherein said Cas9 protein-transcriptional activator fusion protein is a dCas9-VP64 fusion protein.

Embodiment 64. The method of embodiments 59 to 63, further comprising a nucleic acid encoding an sgRNA sequence capable of binding to said Cas9 protein.

Embodiment 65. The method of embodiment 64, wherein said sgRNA targets a type I interferon gene or an IL-15 gene.

Embodiment 66. The method of embodiment 64, wherein said sgRNA targets a type I interferon gene and an IL-15 gene.

Embodiment 67. The method of embodiment 65 or 66, wherein said type I interferon is an IFN-α gene or an IFN-β gene.

Embodiment 68. The method of embodiment 65 or 66, wherein said IFN-α gene is an IFN-α1 gene or an IFN-α2 gene.

Embodiment 69. The method of embodiment 65 or 66, wherein said IFN-β gene is an IFN-β1 gene.

Embodiment 70. The method of any one of embodiments 54 to 69, wherein said NK cell is capable of expressing an elevated level of a type I interferon and an elevated level of IL-15 relative to the standard control NK cell.

Embodiment 71. The genetically engineered NK cell of embodiment 70, wherein said type I interferon is an IFN-α gene or an IFN-β.

Embodiment 72. The genetically engineered NK cell of embodiment 70, wherein said type I interferon is an IFN-α1 gene, an IFN-α2 gene or an IFN-β1.

Embodiment 73. A method of preventing recurrence of leukemia in a subject, the method comprising administering to said subject an effective amount of an natural killer (NK) cell capable of expressing an elevated level of a type I interferon or an elevated level of IL-15 relative to a standard control NK cell.

Embodiment 74. The method of embodiment 73, wherein said leukemia is acute lymphoblastic leukemia.

Embodiment 75. A method of treating or preventing an infectious disease in a subject having leukemia, the method comprising administering an effective amount of a natural killer (NK) cell capable of expressing an elevated level of a type I interferon or an elevated level of IL-15 relative to a standard control NK cell.

Embodiment 76. The method of embodiment 75, wherein said leukemia is acute lymphoblastic leukemia.

Embodiment 77. The method of embodiment 75 or 76, wherein said infectious disease is a viral infection.

### EXAMPLES

### Example 1

Goal: We have identified that the MYC oncogene suppresses host anti-cancer immune surveillance by Natural Killer (NK) cells in high-risk B- and T-cell acute lymphoblastic leukemia (B/T-ALL) (Swaminathan et al., Nature Communications, 2020, Duault et al., under review at Blood). We are translating this knowledge to engineer NK cell-based therapies that cannot be suppressed by the leukemia cells. Once developed, we anticipate that our engineered NK cells would be able to kill ALL, persist in ALL patients by overcoming suppression by leukemia cells, and therefore prevent ALL relapse.

Rationale: B/T-ALL are aggressive because of the overexpression of oncogenes including MYC and because of the downstream pathways the oncogenes drive. In principle, targeting the driver oncogene should kill the malignant lymphocytes (lymphoblasts) and cure ALL. However, targeting many oncoproteins like MYC and RAS still poses a significant clinical challenge because of requirement of these proteins (albeit in small amounts) in healthy tissues and because of the lack of ‘druggable’ domains within these proteins. Here, we target the suppressive effects of oncoproteins like MYC on host immunity rather than the oncoprotein itself. Towards this end, we developed a class of anti-cancer immune cells called NK cells as therapies by engineering them to be avoid being suppressed by B- and T-‘lymphoblasts’.

Methodology: To engineer NK cells that cannot be suppressed by B/T-lymphoblasts, we use CRISPR/CAS9 to knockout, CRISPR/dCAS9-KRAB to knock down, and CRISPR/dCAS9-VP64 to overexpress genes in NK cells that we find to be modulated by lymphoblasts. We recently found that lymphoid malignancies suppress the NK cell-mediated clearance of B/T-lymphoblasts by overexpressing MYC, which in turn blocks the production of proteins named Type I Interferons (IFNs) and IL-15 required for NK maturation and its cytotoxic function (Swaminathan et al., Nature Communications, 2020). Recently, we found that the pathway leading to the production of cytotoxic NK cells in also blocked in ALL patients (Duault et al., Blood, under review). To overcome the leukemia-driven suppression of the Type I IFN and IL-15 production, we employ our CAS9 and dCAS9 engineered NK cells to develop an NK cell that produces as well as responds to Type I IFNs. Therapeutic potential of Type I IFN- and IL-15-producing NK cells can be examined for their ability to kill lymphoblasts in vitro and mediate tumor clearance in patient-derived xenografts of B- and T-ALL in vivo.

Innovation: Prior attempts in constitutively expressing CAS9 and dCAS9 in human NK-92 cell line for CRISPR-based therapeutic applications have been unsuccessful. Constitutive expression of dCAS9-VP64 is useful for generating Type I IFN- and IL-15-producing NK cells for therapeutic purposes. Our methodology also has advantages over traditional lentiviral approaches of overexpressing Type I IFNs and IL-15: (1) it is technically easier to introduce multiple guide RNAs to modulate all Type I IFNs and 11-15 simultaneously as opposed to individually introducing large vectors encoding each Type I IFN and the IL-15 gene, and (2) our engineered NK-92 cells can be seamlessly modified to overexpress genes other than Type I IFNs and IL-15 (eg. NK activating receptors) to develop clinical-grade NK cell-based therapies against high-risk B/T-ALL. Since MYC, whose overexpression causes NK suppression (Swaminathan et al., Nature Communications, 2020), is a key oncogenic driver of not just ALL, but also other blood cancers and solid tumors as well, our engineered NK-92 cells have applications for treating a broad spectrum of aggressive malignancies.

Applicant has comprehensively profiled NK cells in high-risk human B/T-ALL), and has established humanized mice to identify deficits in NK surveillance in ALL. Currently, we are engineering using CRISPR, the NK-92 lymphoblast cell line, which has been extensively manipulated for developing CAR-NK cells and has shown clinical efficacy in acute myeloid leukemia (AML) patients. NK cells derived from peripheral blood of healthy donors or from cord blood can also be used.

For the generation of stable NK-92 cell lines that express CAS9, dCAS9-VP64 and dCAS9-KRAB, the following materials were used:

NK-92 cell line was received from Dr. Jianhua Yu's lab. NK-92 cells were purchased from the American Type Culture Collection (ATCC, ATCC® CRL-2407™)

Recombinant human IL-2 used as a growth factor for NK-92 cells were procured from Peprotech (#200-02).

Plasmids: Lenti-CAS9_EGFP (#63592), dCAS9-VP64_GFP (#61422), pCAG-dCAS9-KRAB-2A-EGFP (#92396) were obtained from Addgene.

Guide RNA expression vectors (ipUSEPR and sgRFP_ipUSEPR) were obtained from Dr. Chun-wei Chen's lab. The ipUSEPR vector is originally cloned by modifying the LentiGuide plasmid by Dr. Chen during his tenure as a postdoctoral fellow in Dr. Scott Armstrong's group at DFCI/Harvard. The sgRFP was developed in the Chen lab at the City of Hope by selecting the best RFP targeting sequence for CRISPR suppression of the RFP signal.

Polybrene used for viral transduction were purchased from Fisher Scientific (#TR1003G).

Lenti-X™ 293T cells for virus production were obtained from Dr Markus Müschen's lab. Müschen lab has purchased these cells from Takara Bio (#632180).

Lipofectamine 2000 used for transfection of Lenti-X 293T cells were purchased from ThermoFisher Scientific (#11668030)

Lenti-X concentrator for lentiviral culture supernatant concentration purchased from Takarabio (#631231).

Sodium butyrate used for the induction virus production in Lenti-X 293T cells were obtained from Fisher Scientific (#AAA1107922).

Sterile Disposable Vacuum Filter Units used for lentiviral culture supernatant filtration were purchased from Fisher Scientific (#SE1M003M00).

DMEM (#10564029) and RPMI-1640 (#72400047) media used for cell culture were purchased from ThermoFisher Scientific.

### Example 2

Materials and Methods

Plasmids:

Lenti-Cas9-EGFP (#63592), dCas9-VP64-GFP (#61422), pCAG-dCas9-KRAB-2A-EGFP (#92396) were obtained from Addgene, pPAX2, pMD2, ipUSEPR-RFP-puromycin for cloning single guide (sg) RNA to modulate specific genes of interest were obtained from Dr. David Chen's lab

Recombinant human (rh) IL-2 Peprotech (#200-02)

Cells:

NK-92 (Obtained from Dr. Jianhua Yu's lab, originally from American Type Culture Collection (ATCC)), K562 for measuring NK cell cytotoxicity (Obtained from Dr. Nora Heisterkamp's lab, originally from ATCC)

Reagents for lentivirus production and transduction:

Polybrene (MilliporeSigma #TR1003G), Lipofectamine 2000 (ThermoFisher Scientific #11668030), Lenti-X concentrator (Takarabio #631231), Sodium butyrate (Fisher Scientific #AAA1107922), Sterile Disposable Vacuum Filter Units (Fisher Scientific #SE1M003M00), DMEM (ThermoFisher Scientific #10564029)

Reagents and supplies for cell culture:

RPMI-1640 (ThermoFisher Scientific #72400047), 150 mm culture dish (Fisher Scientific #FB012925), 15 ml conical centrifuge tubes (Genesee #28-103), 50 ml conical centrifuge tubes (Genesee #28-106), 12 well culture plate (Genesee #25-106), 48 well culture plate (Genesee #25-108), 96 well round bottom culture plate (#25-221), Lenti-X 293T cell line (Takarabio #632180), Fetal bovine serum (FBS) (ThermoFisher Scientific #26140079), 2-Mercaptoethanol (ThermoFisher Scientific #21985023), Round-Bottom Polypropylene Test Tubes (Fisher Scientific #14-959-11B), BsmBI-v2 (NEB #R0739S), Standard T4 ligase (NEB #B0202S), NEB 5-alpha Competent E. coli (NEB #C2987U), Sodium pyruvate (ThermoFisher Scientific #11360070), non-essential amino acids solution (ThermoFisher Scientific #11140050), Penicillin-Streptomycin (ThermoFisher Scientific #15140122), Cell trace violet (ThermoFisher Scientific #C34557), 7-AAD cell viability solution (Biolegend #420403), Syber green master mix (Applied biosystem #A25742), NucleoSpin RNA purification kit (MN #740955.50), SuperScript IV first strand synthesis system (Invitrogen #18091050), Puromycin (Gibco #A11138-03).

CRISPR Guide RNA Cloning:

Guide RNAs were designed with 5′CACCG-forward oligo-3′ and 5′AAAC-reverse oligo-C3′ overhang sequences to clone into BsmBI digested ipUSEPR-RFP-puromycin plasmid. Forward and reverse oligonucleotides were reconstituted in nuclease free water at a concentration of 100 μM, and then mixed in equal proportions. This mixture was heated at 95° C. for 10 min and then temperature was ramp down to 25° C. at 0.5° C./min to anneal the oligonucleotides. Annealed oligonucleotides were cloned into ipUSEPR-RFP-puromycin backbone using T4 DNA ligase.

Lentivirus Production:

Lenti-X 293T cells were grown in 150 cm culture dish to 70-80% confluency and then transfected with PAX2 (20 μg), pMD2 (5 μg) and Lenti-Cas9 EGFP (20 μg) or dCas9-VP64-GFP or dCAS9-KRAB-GFP lentiviral plasmids using lipofectamine 2000 (20p1) in 8 ml serum free DMEM media for 3 hrs at 37° C. After incubation, total cell culture volume was made up to 16 ml by adding 8 ml complete DMEM (contains 100 U/ml penicillin and 100 g/ml streptomycin, 10% FBS, 1× non-essential amino acids and 1 mM sodium pyruvate), media and cells were incubated overnight at 37° C. Next day, media was removed, and cells were treated with sodium butyrate for 5 hrs to enhance lentiviral production. After sodium butyrate induction, media was discarded, and cells were replenished with 16 ml fresh complete DMEM media and incubated at 37 C for 24 hrs. After incubation, lentivirus containing media was filtered using sterile disposable vacuum filter unit and concentrated with Lenti-X concentrator.

Nomenclature Used:

Cas9 (CRISPR associated protein 9), EGFP (Enhanced green fluorescent protein), RFP (Red fluorescent protein), sgRNA (Single guide ribonucleic acid), TL (Interleukin), KRAB (Kruppel associated box), IFN (Interferon), IFAR (Interferon alpha receptor), DNAM-1 (DNAX accessory molecule-1), STAT (Signal transduction and activation of transcription), PD1 (Programmed cell death protein 1), PVRIG (Poliovirus receptor related immunoglobulin domain).

Gene of Interest-Label:

Cas9-EGFP, dCas9-VP64-GFP, dCas9-KRAB-GFP, control sgRNA-RFP, RFP sgRNA-RFP, IL2Ry sgRNA-1-RFP, IL2Ry sgRNA-2-RFP, IL15 sgRNA-RFP.

Lenti-Cas9-EGFP virus concentration using Lenti-X concentrator:

16 ml viral supernatant was concentrated using Lenti-X concentrator (Takara Bio #631231). 4 ml Lenti-X concentrator was mixed with 16 ml viral supernatant and incubated overnight at 4° C. Next day, mixture was centrifuged at 1500×g for 45 min 4° C. After centrifugation, an off-white pellet form at the bottom of centrifugation tube, without disturbing the pellet, supernatant was removed, and off-white pellet was resuspended in 1 ml complete RPMI media and stored at −80° C.

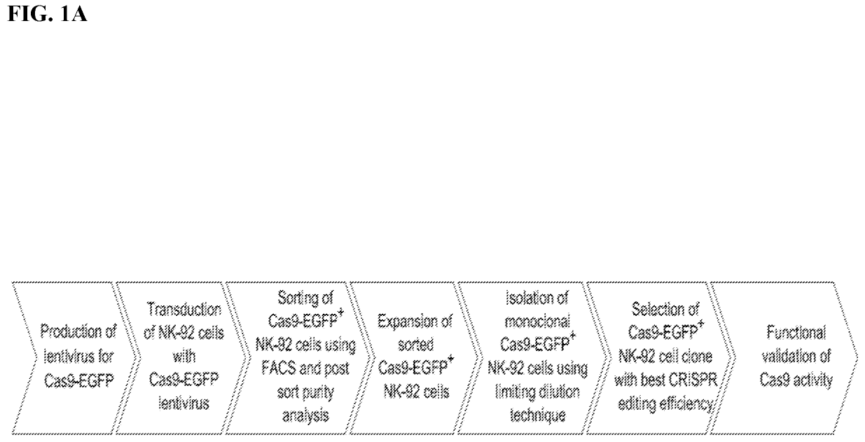

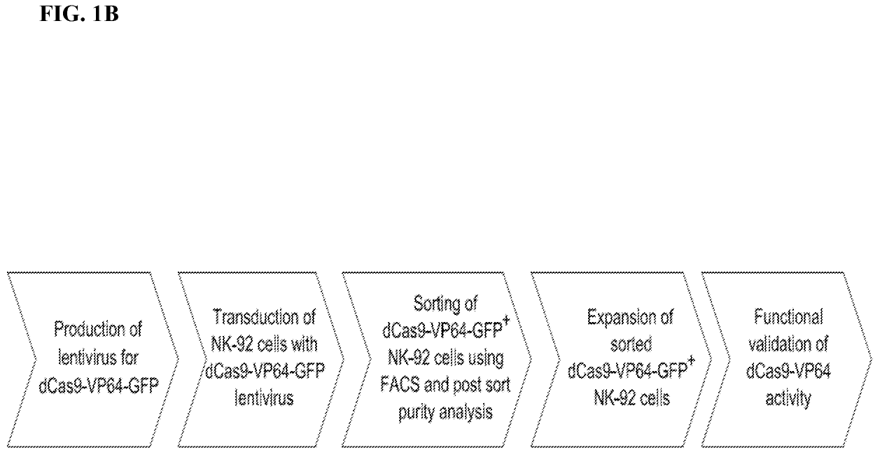

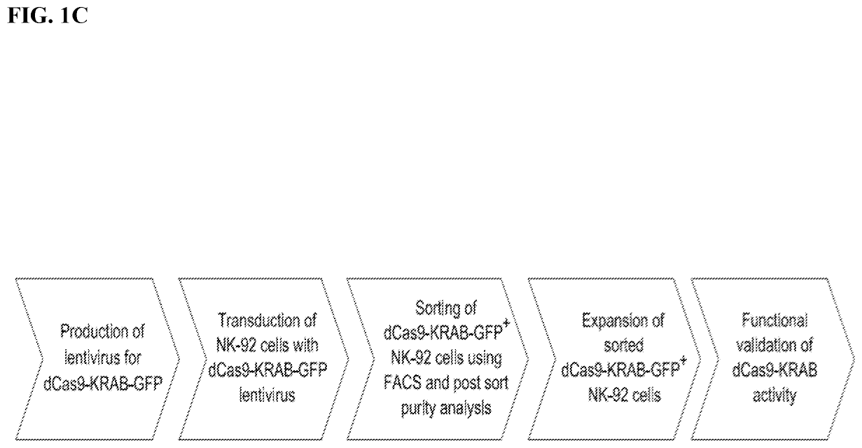

The process of generating and validating stable Cas9-expressing and dCAS9-VP64/dCAS9-KRAB-expressing NK-92 cells is shown in FIGS. 1A-IC.

Generation of stable Cas9-EGFP+ NK-92 cells by lentiviral transduction:

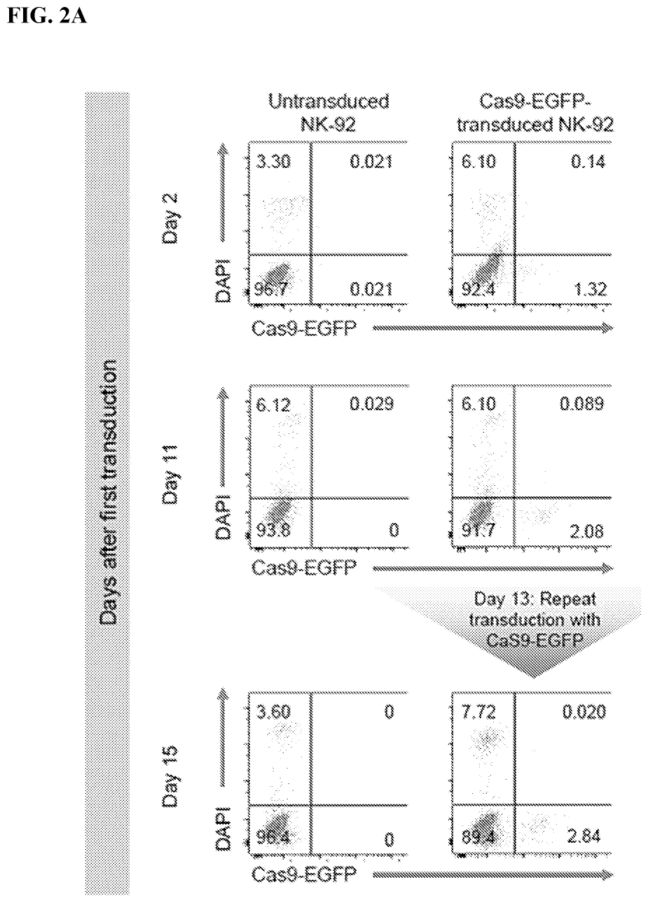

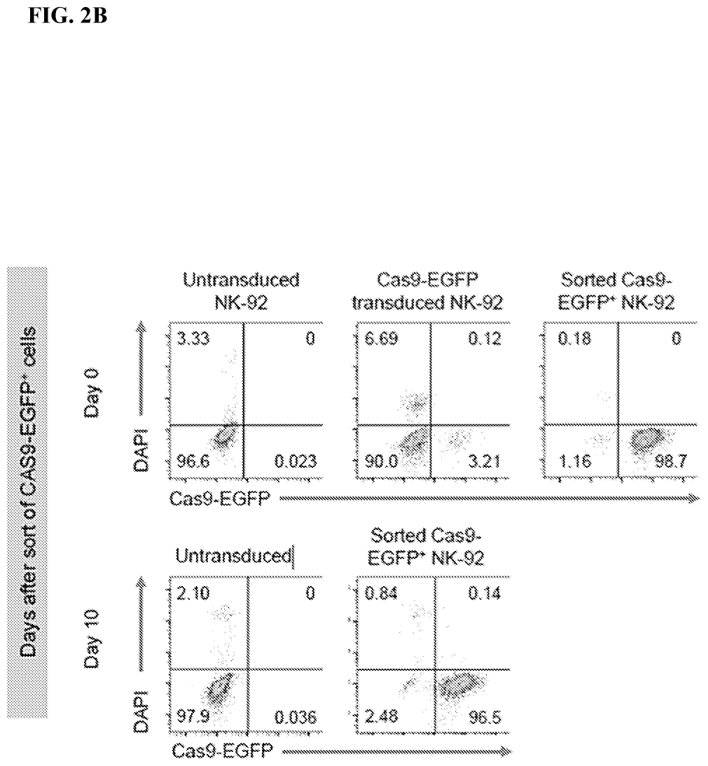

Concentrated Lenti-Cas9-EGFP viral supernatant was rapidly thawed at 37° C., and 500 μl viral supernatant was mixed with 500 μl complete RPMI media (contains 100 U/ml penicillin and 100 g/ml streptomycin, 10% FBS and 1×2-Mercaptoethanol) containing 1.5×106 NK-92 cells. Cells were supplemented with 200 IU of rh IL-2 and 10 μg polybrene and were cultured in 12 well plate at 37° C. for 48 hrs. After incubation, cells were washed and replenished with fresh 2 ml complete RPMI media containing rhIL-2 and analyzed for the percentage of transduced Cas9-EGFP+ NK-92 cells using flow cytometry (FIG. 2A, top panel). On day 11 post transduction, we confirmed that Cas9-EGFP+ NK-92 cells are being maintained in the culture using flow cytometry (FIG. 2A, middle panel). To improve transduction efficiency, on day 13, all Cas9-EGFP+ NK-92 cells were resuspended in 500 μl media and retransduced with 500 μl lentivirus for 48 hrs in the presence of rhIL-2 and polybrene. After 2nd transduction, cells were washed and % of transduced cells were determined using flow cytometry (FIG. 2A, bottom panel). Cells were then expanded in rIL-2 containing RPMI media for 4 days and EGFP+ cells were sorted using Aria Fusion 2 (BD) flow cytometer (FIG. 2B, top panel). Sorted Cas9-EGFP+ NK-92 cells were expanded in complete RPMI media supplemented with rhIL-2 for 10 days and were analyzed for the % of EGFP+ cells to verify sorting purity (FIG. 2B—bottom panel).

Isolation of monoclonal Cas9-EGFP⁺ NK-92 cells using limiting dilution technique:

Cas9-EGFP⁺ NK-92 cells were counted using hemocytometer and cell count was adjusted to 5×10³/ml. The final cell counts to seed 96 well was 5 cells/ml, and to achieve this count, 250 μl of 5×10³/ml solution was mixed with 49.75 ml of complete RPMI media containing rhIL-2. The 100 μl of 5 cells/ml solution was transferred to each well of 96 well round bottom plates and incubated at 37° C. Cells were carefully monitored over a period of two weeks, when single cells clumps grown bigger in size, cells were removed from the 96 well plates, washed, and transferred to 48 well culture plate. After three days of culture in 48 well plate, % of Cas9-EGFP⁺ cells were analyzed using flow cytometry (FIG. 3) and cells were then transferred to T25 flask along with fresh media and rhIL-2. Cells were expanded in T-25 flask and then cryopreserved. Of note, clones 1-4 eventually died out, and could not be used for further experiments.

Selection of Cas9-EGFP⁺ NK-92 cell clone with best CRISPR editing efficiency:

Cas9 activity in monoclonal Cas9-EGFP⁺ NK-92 cells (clone 5 to clone 9) was evaluated using sgRNA expression plasmid ipUSEPR-RFP-puromycin (hereafter termed ‘sgRNA-RFP’). Cas9-EGFP⁺ NK-92 single cell clones were transduced with lentivirus harboring either negative control sgRNA (hereafter termed ‘control sgRNA-RFP’) or sgRNA against RFP (hereafter termed ‘RFP sgRNA-RFP’). Of note, we expect to observe that transduction with ‘RFP sgRNA-RFP’ reduces frequencies of RFP-expressing Cas9-EGFP⁺ NK-92 cells as compared to cells transduced with ‘control sgRNA-RFP’.

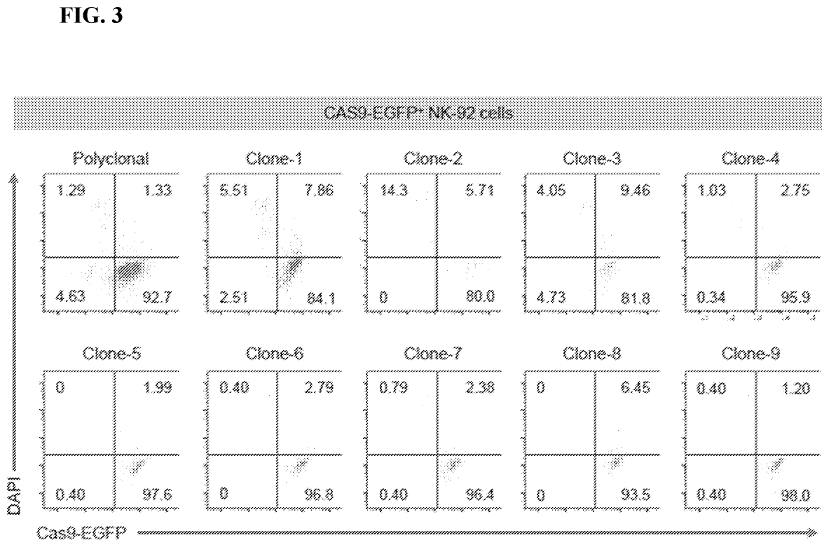

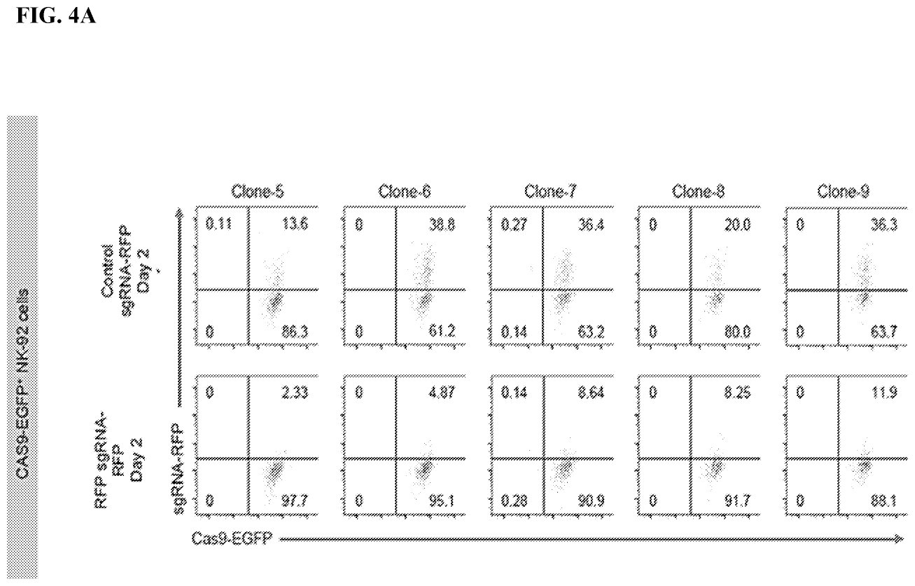

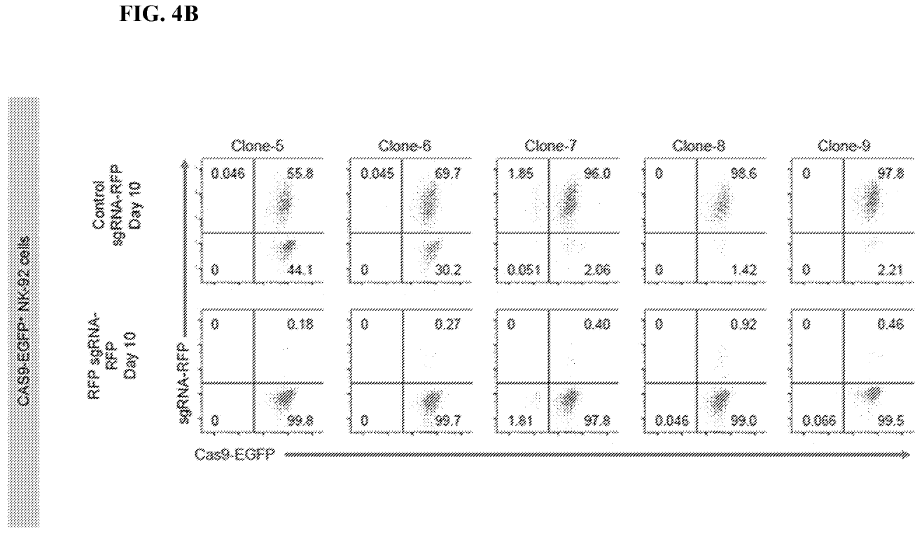

For the above lentiviral transduction with ‘control sgRNA-RFP’ or ‘RFP sgRNA-RFP’, 1.5×10⁶ monoclonal Cas9-EGFP⁺ NK-92 cells were resuspended in 500 μl complete RPMI media and were mixed with 500 μl of concentrated (from 16 ml viral supernatant) ‘control sgRNA-RFP’ lentivirus or ‘RFP sgRNA-RFP’ lentivirus. These cells were also supplemented with rhIL-2 (200 IU) and polybrene (10 g/ml) to support the growth and transduction of monoclonal Cas9-EGFP⁺ NK-92 cells, respectively. 48 hrs after transduction, cells were washed, and percentages of transduced cells were analyzed by flow cytometry (FIG. 4A). sgRNA transduced cells were then selected in puromycin (0.5 g/ml) containing media for 10 days and were analyzed by flow cytometry (FIG. 4B). All the clones tested showed equivalent Cas9 editing efficiency with <1% RFP⁺ cells after puromycin selection in the ‘RFP sgRNA-RFP’ group (FIG. 4).

Validation of IL2Rγ knockout in Cas9-EGFP⁺ NK-92 cells using sgRNA against IL2Rγ:

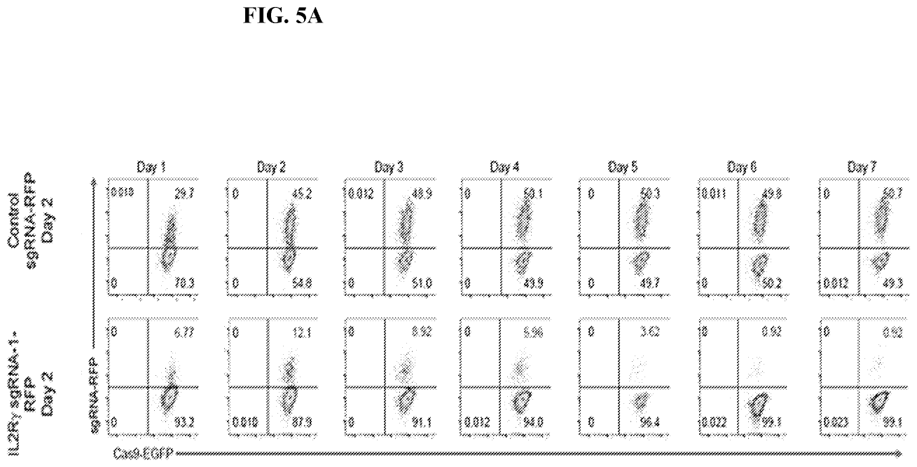

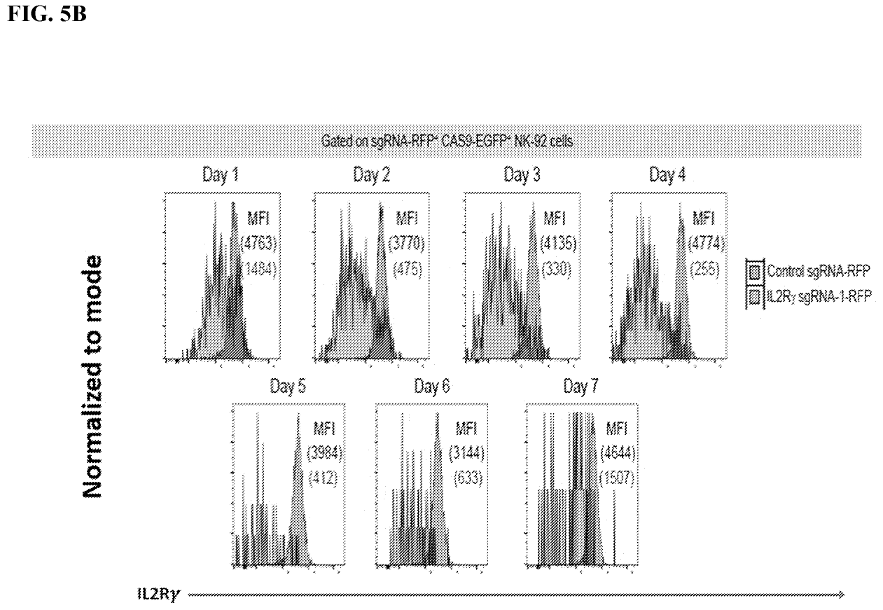

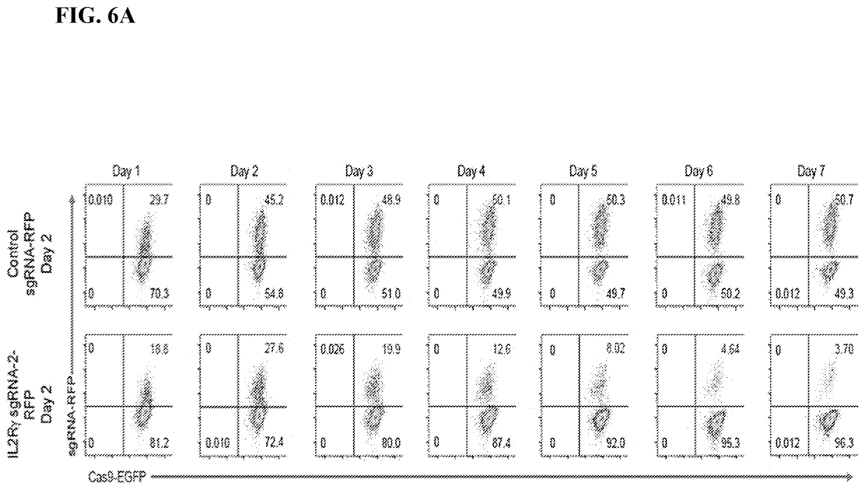

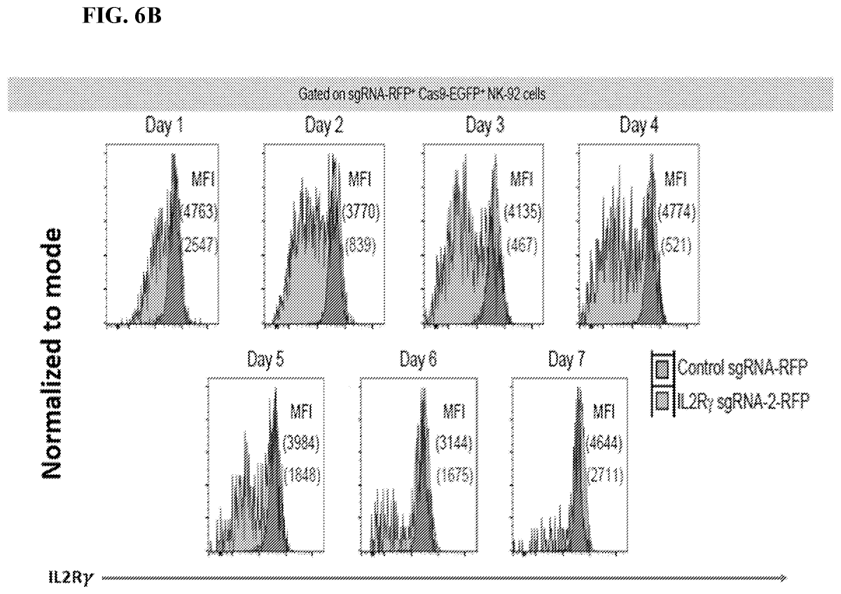

To further validate the Cas9-mediated knockout in monoclonal Cas9-EGFP⁺ NK-92 cells, we knocked out the IL-2Rγ chain using two different guides against the IL-2Rγ chain (termed ‘IL2Rγ sgRNA-1-RFP’ and ‘IL2Rγ sgRNA-2-RFP’). NK-92 cell line is dependent on IL-2 for growth and proliferation. Therefore, we postulated that knocking out IL-2R signaling subunits will induce cell death even the presence of exogenous IL-2. We compared the changes in frequencies of sgRNA-RFP⁺ cells between Cas9-EGFP⁺ NK-92 cells carrying either ‘control sgRNA-RFP’ or ‘IL2Rγ sgRNA-RFP’. After 48 hrs of transduction, cells were washed and analyzed daily for 7 days for frequencies of sgRNA-RFP⁺ cells, as well as the surface expression of IL-2Rγ chain on sgRNA-RFP⁺ Cas9-EGFP⁺ cells by flow cytometry. We found a reduction in frequencies of sgRNA-RFP⁺ population in cells carrying the IL2Rγ sgRNA as compared to cells harboring the control sgRNA (FIGS. 5A and 6A). Furthermore, we found that sgRNA-RFP⁺ Cas9-EGFP⁺ NK-92 cells with IL2Rγ sgRNA exhibited lower surface expression of IL2Rγ as compared to cells carrying the control sgRNA (FIGS. 5B and 6B). These findings validate the successful generation of stable Cas9-EGFP⁺ NK-92 cells for future modifications.

Generation and Validation of Stable dCAS9-VP64-GFP Expressing NK-92 Cells:

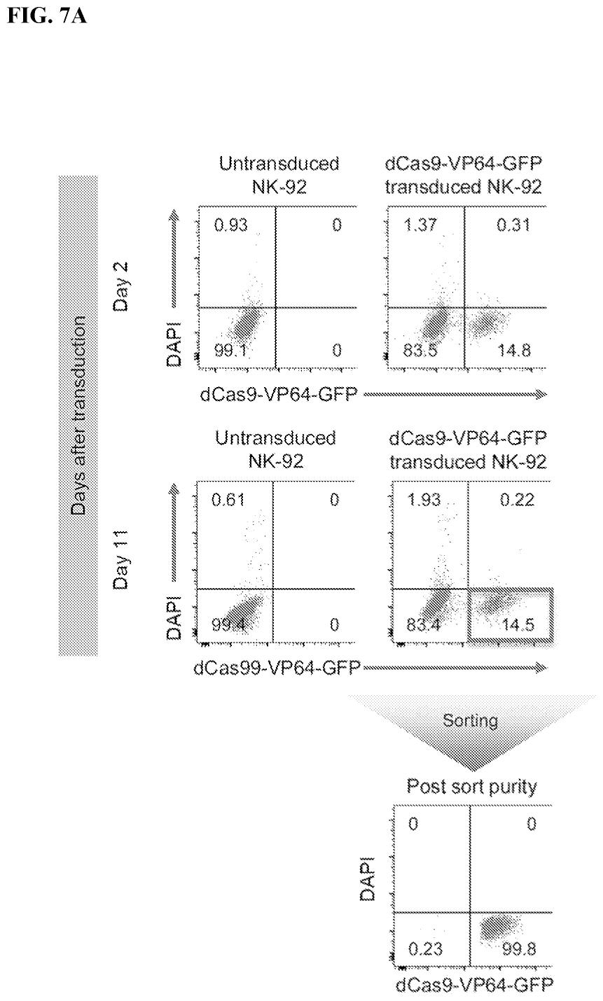

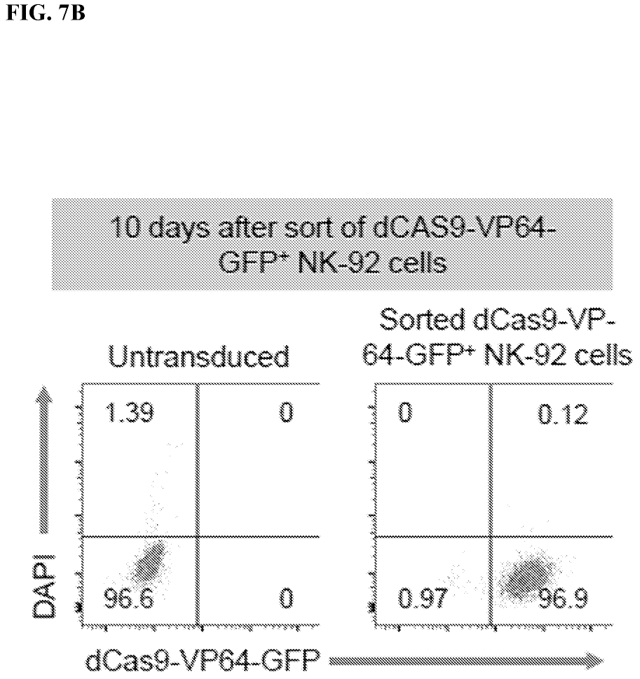

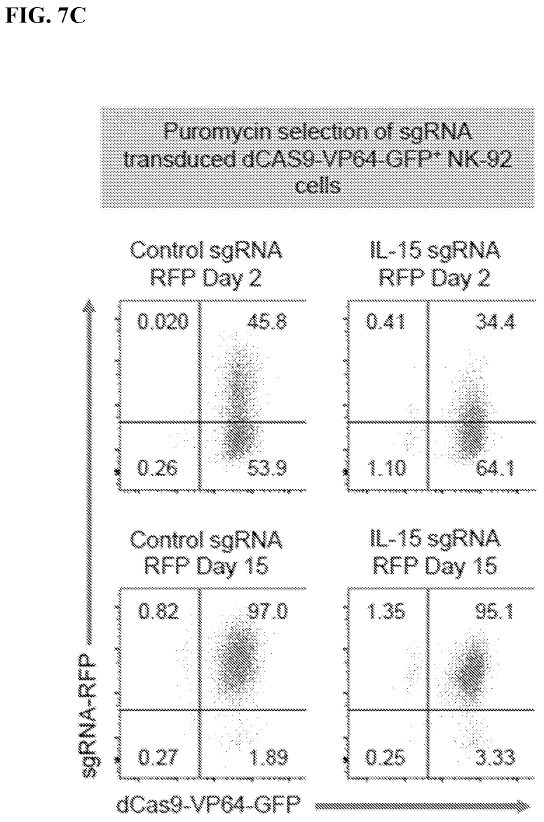

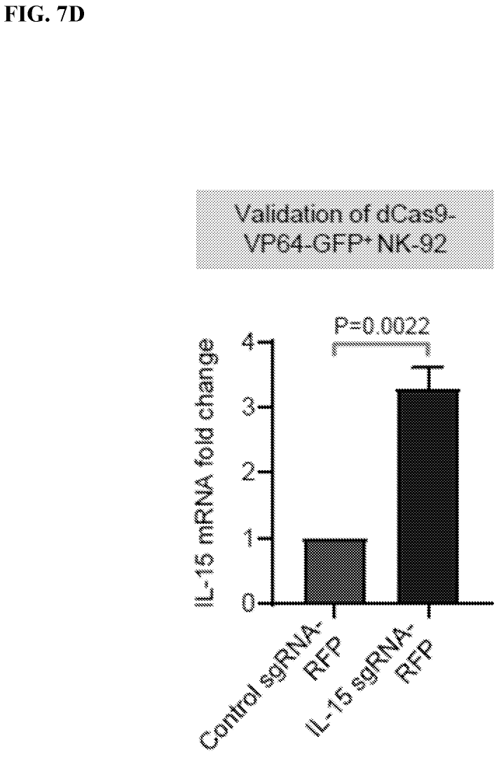

NK-92 cells (1.5×106) were resuspended in 500 μl complete RPMI media and were mixed with 500 μl concentrated dCAS9-VP64-GFP lentivirus. Cells were also supplemented with rhIL-2 and polybrene to support growth and transduction respectively. After 24 hrs of transduction, cells were washed and replenished with fresh complete RPMI media and rhIL-2. After 48 hrs, cells were analyzed by flow cytometry to measure the percentages of transduced cells (FIG. 7A top panel). Cells were expanded for 11 days with media and rhIL-2 change on every third day. dCAS9-VP64-GFP transduced cells were then sorted using FACS method and post sort-purity was 99.8% (FIG. 7A middle and bottom panel). Sorted dCas9-VP64-GFP+ NK-92 cells were expanded in complete RPMI media supplemented with rhIL-2 for 10 days and were analyzed for the percentage of EGFP+ cells to verify sorting purity (FIG. 7B).

To validate the dCAS9-VP64 mediated transcriptional activation in NK-92 cells, we selected IL-15 gene. IL-15 is required for the growth and effector cytolytic function of NK cells. We transduced dCAS9-VP64-GFP+ NK-92 cells with control sgRNA-RFP or CRISPRa IL-15 sgRNA (FIG. 7C) designed using Broad institute's web based tool. Transduced cells were selected in puromycin, RNA was isolated by NucleoSpin RNA purification kit and cDNA was prepared using SuperScript IV first strand synthesis system. Gene expression was quantified by qPCR and fold difference was determined using comparative 2-AACT method. We observed ˜3-fold increase in the levels of IL-15 transcripts in dCAS9-VP64-GFP+ NK-92 cells containing the IL-15 sgRNA-RFP as compared to their counterparts transduced with control sgRNA-RFP (FIG. 7D). These findings validate the successful generation of stable dCas9-VP64-GFP+ NK-92 cells for future modifications.

Generation of Stable dCAS9-KRAB-GFP Expressing NK-92 Cells:

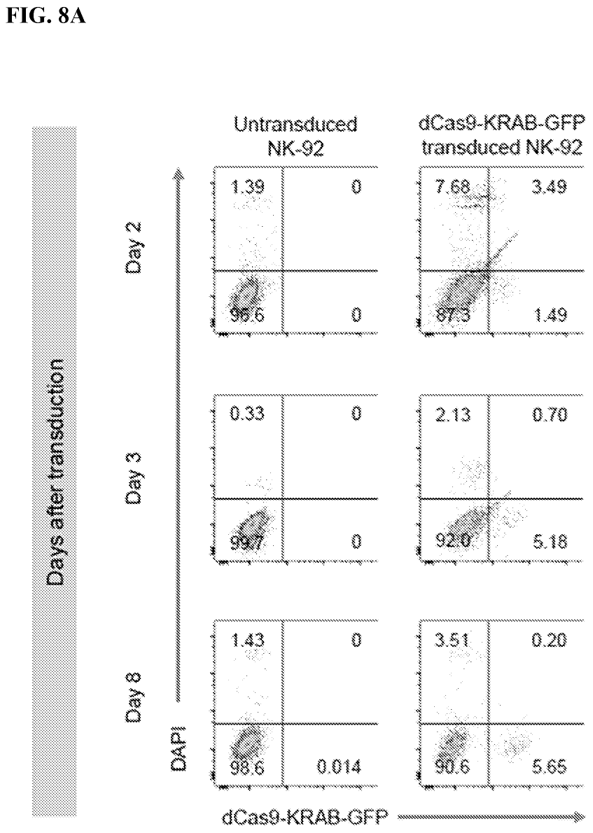

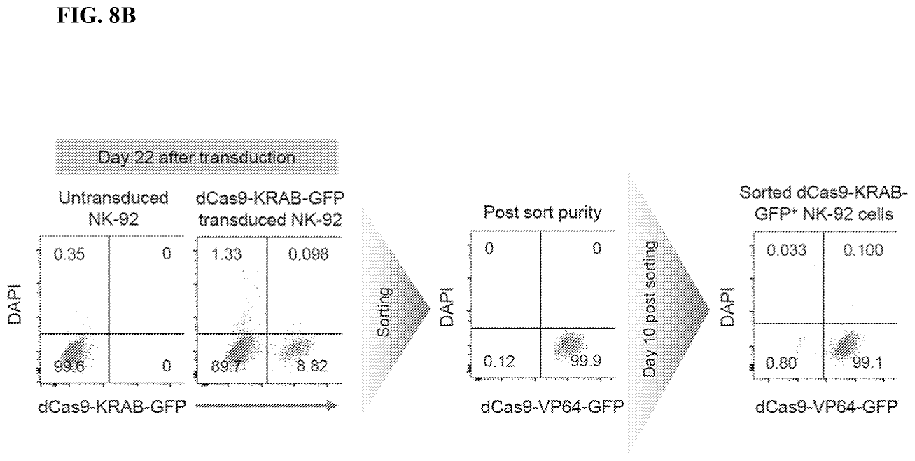

NK-92 cells were transduced with dCAS9-KRAB-GFP lentivirus as described in the earlier section (refer to generation of dCAS9-VP64-GFP⁺ NK-92 cells). After transduction, cells were analyzed for the percentage of transduced cells on day 2, day 3 and day 8 (FIG. 8A). Cells were expanded over a period of three weeks in complete RPMI media supplemented with rhIL-2 with media containing IL-2 being changed every 3 days. On day 22, cells were sorted, post sort purity was determined on the day of sorting (99.9%) and after 10 days of expansion of sorted cells (99.1%) (FIG. 8B). Cells were then cryopreserved for future use.

Analysis of Cytotoxic Potential of CAS9, dCAS9-VP64 and dCAS9-KRAB Transduced NK-92 Cells:

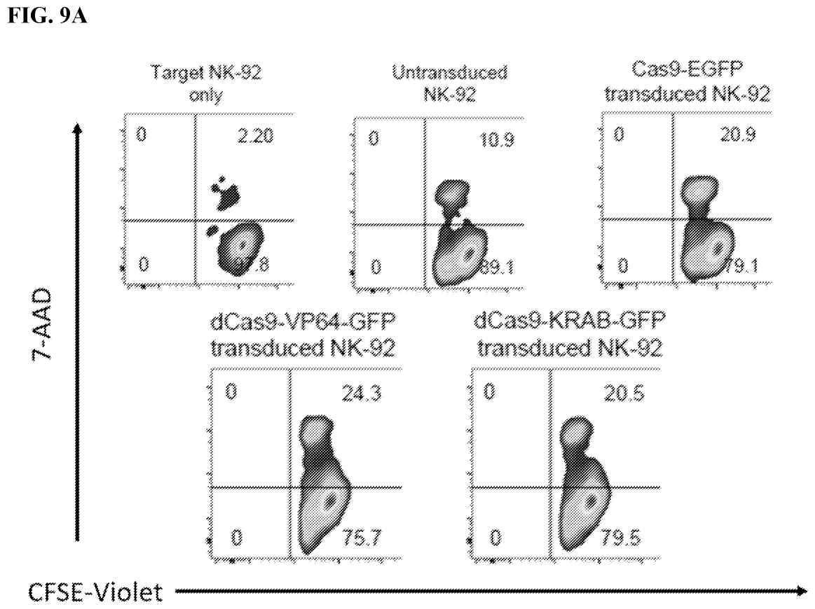

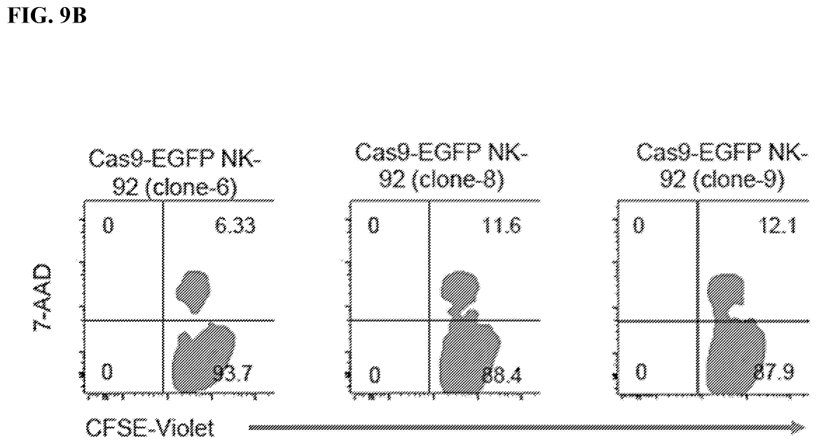

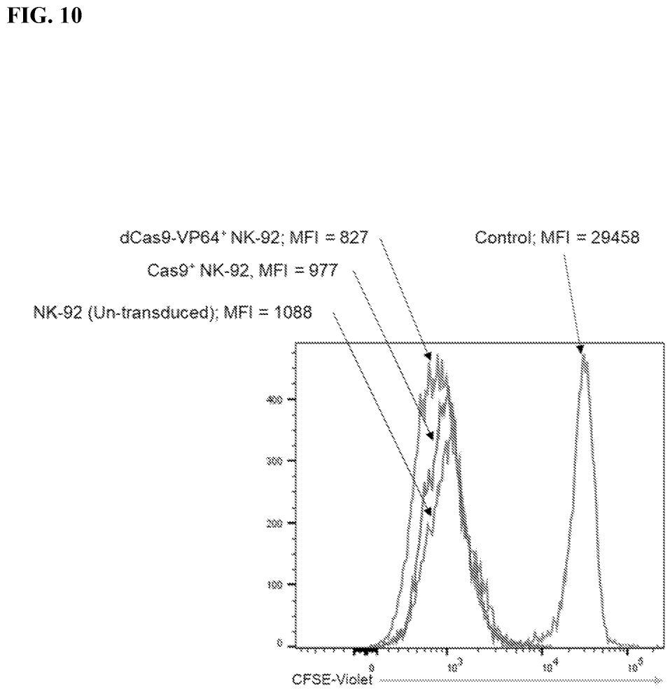

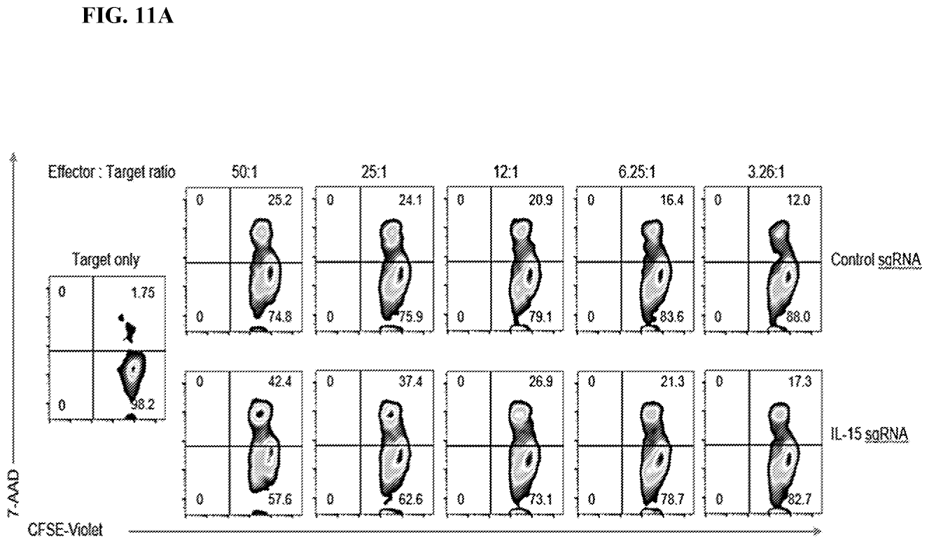

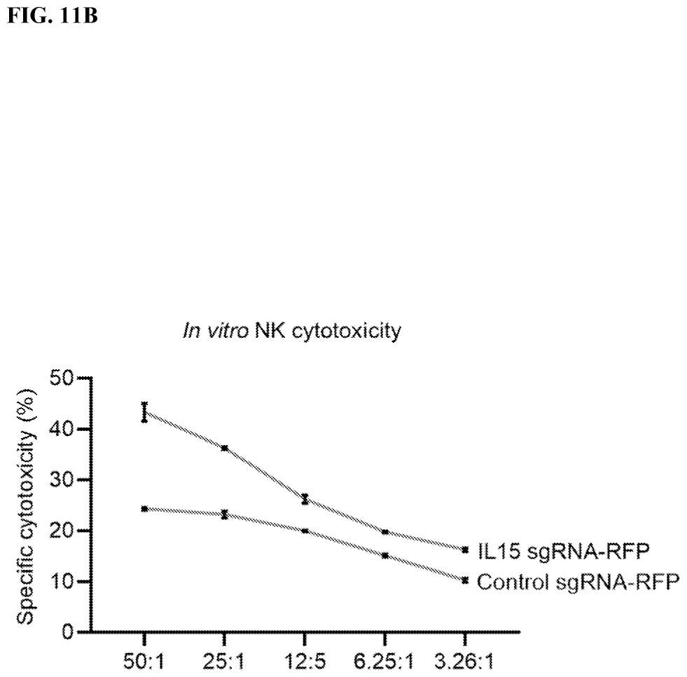

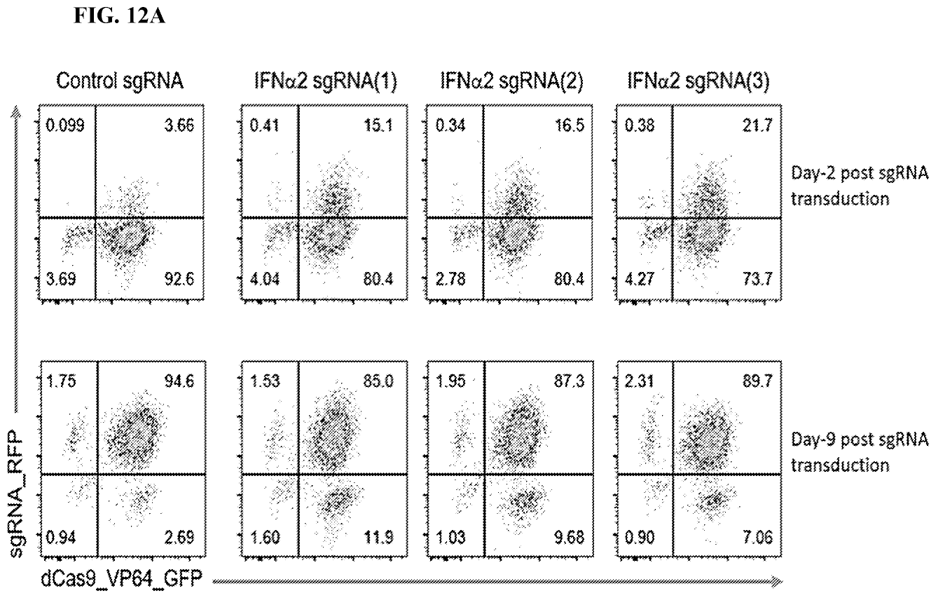

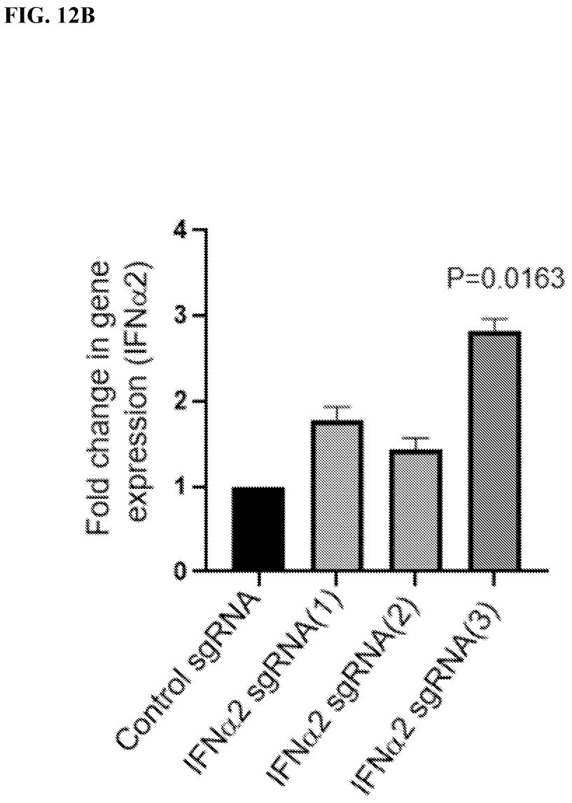

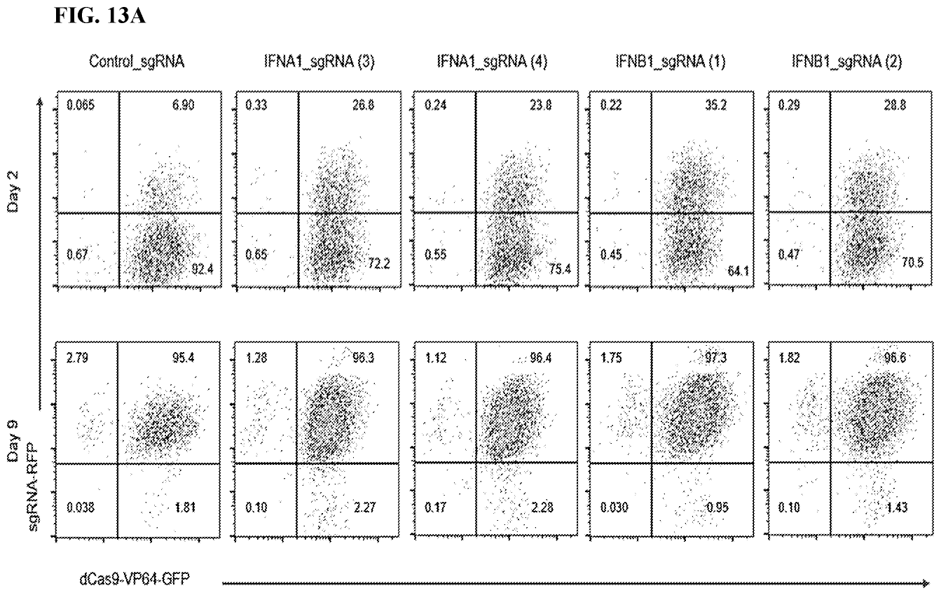

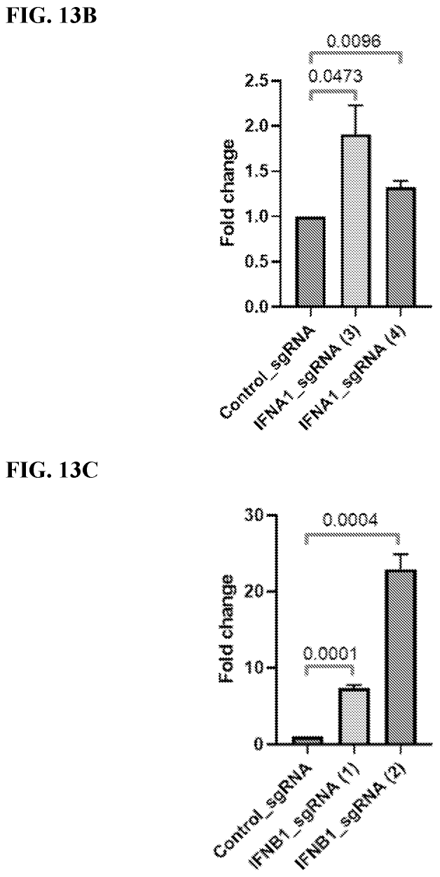

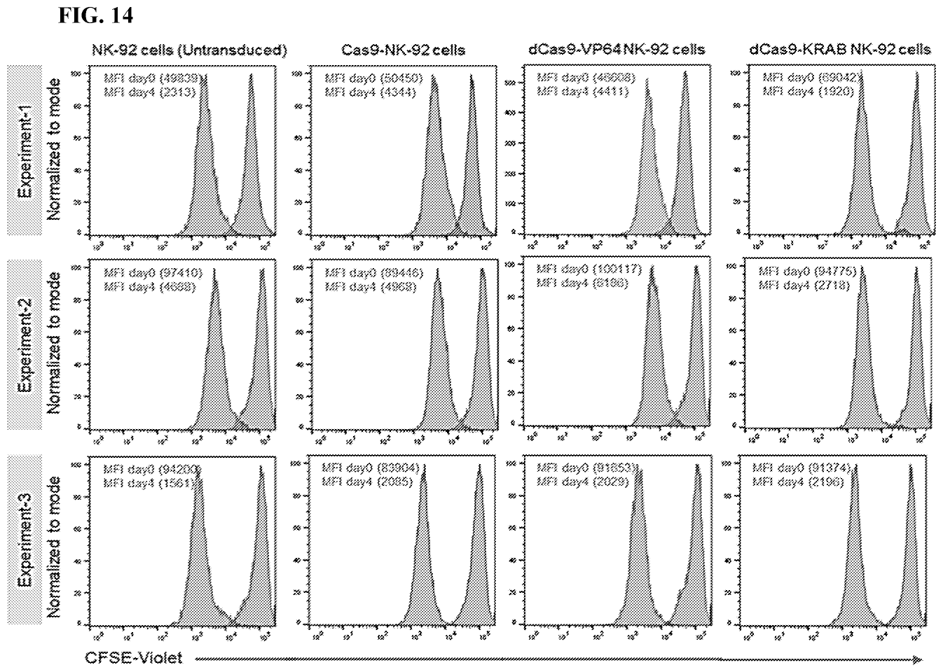

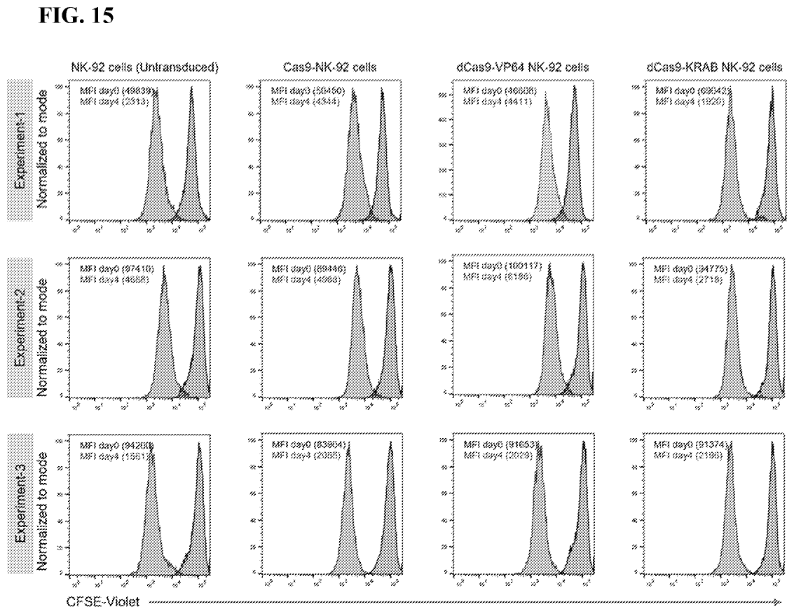

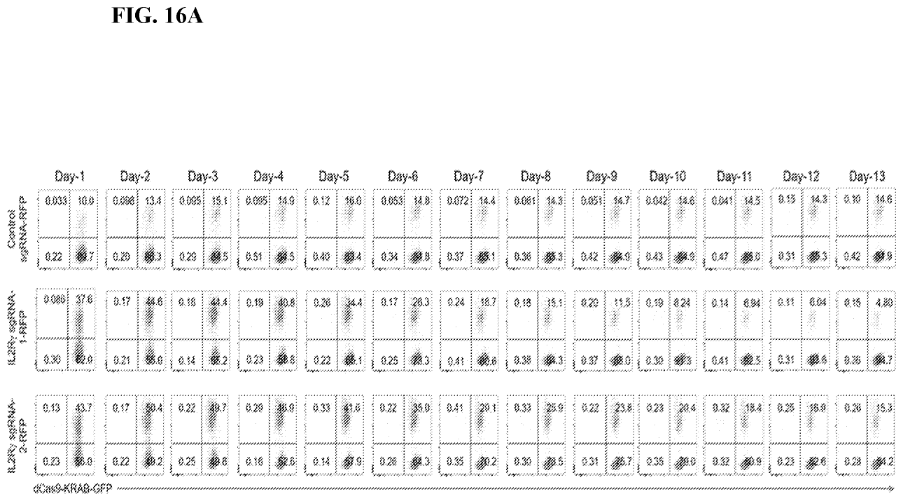

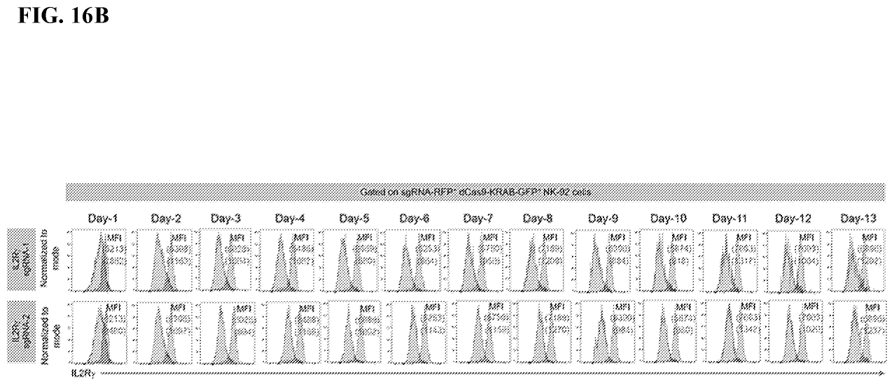

To evaluate the cytotoxic functions of transduced NK-92 cells, human erythroleukemia K562 cell line was used as a target cell to measure NK cell lytic activity. To distinguish NK-92 effector cells from target cells, K562 cells were labelled with 2.5 μM CFSE-violet dye for 30 min in phosphate buffered saline at 37° C. Cells were then washed with complete RPMI media two times and were co-cultured with K-562 cells at 10:1 (Effector:Target) in U bottom 96 well plate for 5 hrs. For control group, labelled K-562 cells were cultured without NK-92 cells to measure the cell death without NK cells. After incubation, cells were stained with 7-AAD, and cell-death of K562 cells were analyzed using flow cytometry. We did not observe an impairment of NK cell-mediated clearance of target leukemia cells in NK-92 cells engineered to constitutively overexpress CAS9 and dCAS9 systems (FIG. 9A). These findings suggest that CAS9 and dCAS9-modified NK cells are suitable for developing genetically engineered NK cell-based therapies. We have also analyzed the cytotoxicity of Cas9-NK-92 single cells clones (Clone-6,-8,-9) and found that Clone-6 had slightly lower cytotoxicity when compared to the untransduced NK-92 cells (FIG. 9B). This result suggests an intra-clonal variation in the cytotoxicity potential of monoclonal Cas9 NK-92 cells and require prior screening of clones before conducting any pilot scale gRNA library screening experiments.

A List of Genes that Regulate NK Cell-Mediated Host Immunity in MYC-Driven ALL Will be Manipulated by Us in the CAS9- and dCAS9-Expressing NK-92 Cells:

Guides were designed using Broad institute's web based tool available at: portals.broadinstitute.org/gpp/public/analysis-tools/sgrna-design.

TABLE 1

| Gene name | CRISPR system |
|---|---|
| IL-15 | dCAS9-VP64 |
| IFNα2 | dCAS9-VP64 |
| IFNα1 | dCAS9-VP64 |
| IFNβ1 | dCAS9-VP64 |
| STAT1/2 | dCAS9-VP64 |
| | CAS9/dCAS9-KRAB - as negative control |
| IFNAR 1/2 | dCAS9-VP64 |
| | CAS9/dCAS9-KRAB - as negative control |
| NKG2D | dCAS9-VP64 |
| | CAS9/dCAS9-KRAB - as negative control |
| NKp46 | dCAS9-VP64 |
| | CAS9/dCAS9-KRAB - as negative control |
| PD1 | CAS9/dCAS9-KRAB |
| DNAM-1 | CAS9/dCAS9-VP64 - to be determined based on function |
| | of the molecule in ALL patient samples |
| PVRIG | CAS9/dCAS9-KRAB - to be determined based on |
| | function of the molecule in ALL patient samples |
| CD96 | CAS9/dCAS9-VP64 - to be determined based on function |
| | of the molecule in ALL patient samples |

### Example 3

The methods described in Examples 1 and 2 will be applied to other Natural Killer (NK) cell lines. Such other NK cell lines may comprise the cell lines described in patent applications WO 2018/056736, WO 2019/182422, and WO 2019/182425, hereby included by reference. Other NK cell lines may also comprise NK cell lines described in the literature, such as the NK-101 cell line as described by Yang H G et al. (Yang H G, Kang M C, Kim T Y, et al. Discovery of a novel natural killer cell line with distinct immunostimulatory and proliferative potential as an alternative platform for cancer immunotherapy. J Immunother Cancer. 2019; 7(1):138. Published 2019 May 24. doi:10.1186/s40425-019-0612-2).

### Example 4

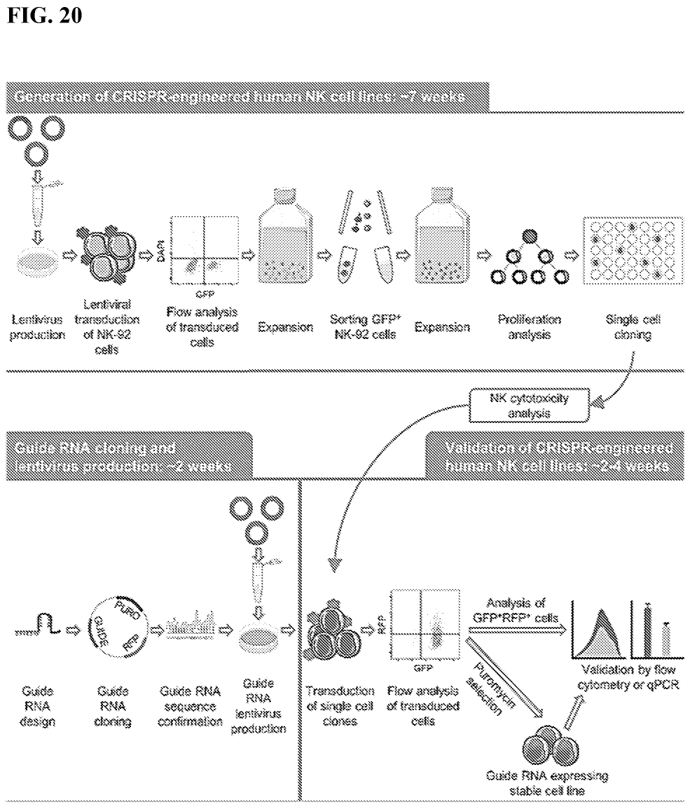

Cytotoxic natural killer cells kill tumors and infected cells. CRISPR-based gene editing and transcriptional regulation were carried out in hard-to-manipulate NK-92 cells. NK-92-based therapies were found to be safe and efficacious in preclinical studies of cancers. Here, pioneering of the generation and validation of NK-92 cells were performed constitutively, expressing Cas9 or dCas9 for knockout (CRISPRko), transcriptional activation (CRISPRa), or transcriptional repression (CRISPRi) of genes (FIG. 20). CRISPR-engineered NK-92 cell platforms might be modified for research and off-the-shelf therapeutic applications.

The following cell culture protocol was about 10-15 days for NK-92 cells, about 3 days for K-562 cells, and about 3-6 days for HEK cells.

The NK-92 natural killer (NK), chronic myeloid leukemia K-562, and Lenti-X293T human embryonic kidney (HEK) cell lines were thawed at 37° C. in a metallic bead bath. (See material and equipment section for details of NK-92, K-562, and HEK cell culture growth media). Thawed NK-92 and K-562 cells were transferred into two separate 15 mL tubes containing 10 mL each of complete RPMI media. HEK cells were transferred into a single 15 mL tube containing 10 mL complete DMEM medium. Cells were pelleted down by centrifugation at 300×g for 5 min at room temperature (RT; 18-22° C.) and the supernatant was aspirated. The NK-92, K-562, and HEK cells were washed again with 10 mL of their respective media for completely removing DMSO (300×g, 5 min at RT).

The NK-92 cells were then resuspended in 1 mL of complete RPMI media, cells were counted and seeded 0.5-1×10⁵ cells/200 μl/well in a 96-well plate (flat bottom) in NK cell media (complete RPMI+rhIL2) at 37° C. When cells became confluent (media color changes to light yellow), spin down the cells (300×g at RT for 5 min), media was aspirated. Cells were resuspended in 500 μl of fresh NK cell media and culture them in a 48-well plate at 37° C. After cells were confluent, they were spun down, media was aspirated, and these cells were transferred to a T-25 flask containing 5 mL of fresh NK cell media. After expansion in a T-25 flask for 3 days, cells were ready to be used in transduction experiments.

For successful NK cell cytotoxicity analyses, K-562 cells that have been in culture for at least 3 days are used after thawing. Once thawed, culturing K-562 cells were not recommended beyond 3 months.

HEK cells were resuspended in 1 mL of complete DMEM media, the cells were counted, and ˜5×10⁵ cells were seeded in a 150 mm culture dish in 15 mL complete DMEM.

For successful lentiviral transduction, HEK cells 1-2 passages (˜3-6 days) were recommended after thaw. Once thawed, culturing HEK cells were not recommended beyond 5 passages.

TABLE 2
Reagents and Resources

| REAGENT or RESOURCE | | |
|---|---|---|
| Antibodies | SOURCE | IDENTIFIER |
| APC anti-human CD132 (IL2Rγ); | Biolegend | Cat# 338607, RRID: |
| working dilution 1:100 | | AB 2123585 |

Bacterial and virus strains

| | | |
|---|---|---|
| NEB 5-alpha Competent E. coli | New England Biolabs | Cat# C2987U |

Critical commercial assays

| | | |
|---|---|---|
| GeneJET gel Extraction Kit | Thermo Fisher Scientific | Cat# K0691 |
| GeneJET Plasmid Miniprep Kit | Thermo Fisher Scientific | Cat# K0502 |
| CellTrace Violet Cell | Thermo Fisher Scientific | Cat# C34557 |
| Proliferation Kit | | |
| NucleoSpin RNA purification kit | Macherey Nagel | Cat# 740955.250 |
| NucleoBond Xtra Maxi Plus EF | Macherey Nagel | Cat# 740426.50 |
| Lenti-X Concentrator | Takarabio | Cat# 631231 |
| SuperScript IV First Strand | Thermo Fisher Scientific | Cat# 18091050 |
| Synthesis System | | |
| Lipofectamine 2000 | Thermo Fisher Scientific | Cat# 11668019 |

Chemicals, peptides, and recombinant proteins

| | | |
|---|---|---|
| Dulbecco's phosphate-buffered | Thermo Fisher Scientific | Cat# 14190144 |
| saline (DPBS), pH 7.4 without Ca²⁺ | | |
| and Mg²⁺ | | |
| Recombinant human interleukin-2 | Peprotech | Cat# 200-02 |
| (rhIL-2) | | |
| Poly-L-Lysine | R&D Systems | Cat# 3438-100-01 |
| Polybrene | Millipore Sigma | Cat# TR-1003-G |
| Sodium Butyrate | Fisher Scientific | Cat# AAA1107922 |
| Sodium azide | Sigma-Aldrich | Cat# S2002-100G |
| Puromycin | Thermo Fisher Scientific | Cat# A1113803 |
| Trypsin/EDTA Solution (TE) | Thermo Fisher Scientific | Cat# R001100 |
| LB Agar, Miller (Pre-Buffered | Fisher Scientific | Cat# BP9734-500 |
| Capsules) | | |
| Ethanol 70% mol. Grade | Fisher Scientific | Cat# BP8201500 |
| Glycerol | Fisher Scientific | Cat# BP2291 |
| Ampicillin | Fisher Scientific | Cat# BP1760-5 |
| Certified Agarose Powder | Bio-Rad | Cat# 1613100 |
| 4,6-diamidino-2-phenylindole | Biolegend | Cat# 422801 |
| (DAPI); working dilution 1:5000 | | |
| 7-AAD Viability Staining Solution | Biolegend | Cat# 420403 |
| (200 tests) | | |
| Penicillin Streptomycin | Thermo Fisher Scientific | Cat# 15140122 |
| 2-Mercaptoethanol | Thermo Fisher Scientific | Cat# 21985023 |
| MEM Non-essential Amino Acids | Thermo Fisher Scientific | Cat# 11140050 |
| (100X) | | |
| GlutaMAX (100X) | Thermo Fisher Scientific | Cat# 35050061 |
| Sodium Pyruvate | Thermo Fisher Scientific | Cat# 11360070 |
| Fetal Bovine Serum (FBS) | Thermo Fisher Scientific | Cat# 26140079 |
| DMEM (Dulbecco's Modified | Thermo Fisher Scientific | Cat# 11965126 |
| Eagle Medium) | | |
| RPMI (Roswell Park Memorial | Thermo Fisher Scientific | Cat# 11875119 |
| Institute)-1640 | | |
| SOS medium | New England Biolabs | Cat# B9020S |
| Terrific Broth (granulated) | Fisher Scientific | Cat# BP9728-500 |
| PowerUp SYBR Green Master Mix | Thermo Fisher Scientific | Cat# A25742 |
| BsmBI-v2 | New England Biolabs | Cat# R0739S |
| CutSmart buffer (10X) | New England Biolabs | Cat# B7204S |
| Standard T4 ligase | New England Biolabs | Cat# B0202S |
| 50X TAE Buffer | Bio-Rad | Cat# 1610743 |
| 1 kb Plus DNA Ladder | New England Biolabs | Cat# N3232S |
| Quick load purple 1 kb DNA ladder | New England Biolabs | Cat# N0552S |
| SYBR Green nucleic acid gel stain | Thermo Fisher Scientific | Cat# S7563 |
| (10000X) | | |
| Gel Loading Dye, Purple (6X), no | New England Biolabs | Cat# B7025S |
| SDS | | |
| HyPure Molecular Grade Water | Cytiva lifesciences | Cat# SH30538.03 |

Experimental models: cell lines

| | | |
|---|---|---|
| NK-92 cell line | ATCC | Cat# CRL-2407 |
| Lenti-X 293T cell line | Takara Bio | Cat# 632180 |
| K-562 cell line | ATCC | Cat# CCL-243 |
| Recombinant DNA | | |
| Lenti Cas9-EGFP | Addgene | Cat# 63592, RRID: |
| | | Addgene_63592 |
| pCAG dCas9-KRAB-2A-EGFP | Addgene | Cat# 92396, RRID: |
| | | Addgene_92396 |
| dCAS9-VP64-GFP | Addgene | Cat# 61422, RRID: |
| | | Addgene 61422 |
| ipUSEPR | From Chun-Wei Chen | House-made plasmid |
| | | (Yang et al., 2021) |
| pMD.2G | Addgene | Cat# 12259, RRID: |
| | | Addgene 12259 |
| psPAX2 | Addgene | Cat# 12260, RRID: |
| | | Addgene 12260 |

Oligonucleotides (5′->3′)

| | | |
|---|---|---|
| Control (Scramble) sgRNA | Derived from (Wang et al., 2014); | GTAGCGAACGTGT |
| (forward); CRISPRko/i/a | ordered form integrated DNA | CCGGCGT (SEQ ID |
| | technologies (IDT) | NO: 1) |
| Control (Scramble) sgRNA | Reverse complement of control | ACGCCGGACACGT |
| (reverse) | sgRNA (forward); CRISPRko/i/a, | TCGCTAC (SEQ ID |
| | (https://www.bioinformatics.org/ | NO: 2) |
| | sms/rev_comp.html) | |
| Red fluorescent protein (RFP) | From Chun-Wei Chen lab | GTCACCACATACG |
| sgRNA (forward); CRISPRko | | AAGACGG (SEQ ID |
| | | NO: 3) |
| RFP sgRNA (reverse) | Reverse complement of RFP | CCGTCTTCGTATGT |
| | sgRNA (forward); CRISPRko, | GGTGAC (SEQ ID |
| | (https://www.bioinformatics.org/ | NO: 4) |
| | sms/rev_comp.html) | |
| IL2RG sgRNA (1) (forward); | https://portals.broadinstitute. | GGTGCAGTACCGG |
| CRISPRko | org/gppx/crispick/public | ACTGACT (SEQ ID |
| | | NO: 5) |
| IL2RG sgRNA (1) (reverse); | Reverse complement of IL2RG | AGTCAGTCCGGTA |
| CRISPRko | sgRNA (1) (forward); CRISPRko, | CTGCACC (SEQ ID |
| | (https://www.bioinformatics.org/ | NO: 6) |
| | sms/rev_comp.html) | |
| IL2RG sgRNA (2) (forward); | https://portals.broadinstitute. | AGCCGCTTTAACC |
| CRISPRko | org/gppx/crispick/public | CACTCTG (SEQ ID |
| | | NO: 7) |
| IL2RG sgRNA (2) (reverse); | Reverse complement of IL2RG | CAGAGTGGGTTAA |
| CRISPRko | sgRNA (2) (forward); CRISPRko, | AGCGGCT (SEQ ID |
| | (https://www.bioinformatics.org/ | NO: 8) |
| | sms/rev_comp.html) | |
| IL2RG sgRNA (1) (forward); | https://portals.broadinstitute. | TGGTAATGATGGC |
| CRISPRi | org/gppx/crispick/public | TTCAACA (SEQ ID |
| | | NO: 15) |
| IL2RG sgRNA (1) (reverse); | Reverse complement of IL2RG | TGTTGAAGCCATC |
| CRISPRi | sgRNA (1) (forward); CRISPRi, | ATTACCA (SEQ ID |
| | (https://www.bioinformatics.org/ | NO: 16) |
| | sms/rev_comp.html) | |
| IL2RG sgRNA (2) (forward); | https://portals.broadinstitute. | AGGGATGTGAATG |
| CRISPRi | gor/gppx/crispick/public | GTAATGA (SEQ ID |
| | | NO: 17) |
| IL2RG sgRNA (2) (reverse); | Reverse complement of IL2RG | TCATTACCATTCA |
| CRISPRi | sgRNA (2) (forward); CRISPRi, | CATCCCT (SEQ ID |
| | (https://www.bioinformatics.org/ | NO: 18) |
| | sms/rev_comp.html) | |
| IL-15 sgRNA (forward); CRISPRa | https://portals.broadinstitute. | ACACCTCCCGCGG |
| | org/gppx/crispick/public | AGACTGG (SEQ ID |
| | | NO: 9) |
| IL-15 sgRNA (reverse); CRISPRa | Reverse complement of IL-15 | CCAGTCTCCGCGG |
| | sgRNA (forward); CRISPRa, | GAGGTGT (SEQ ID |
| | (https://www.bioinformatics.org/ | NO: 10) |
| | sms/rev_comp.html) | |
| GAPDH (forward); qPCR | Derived from (Kooreman et al., | GTCTCCTCTGACTT |
| | 2017) | CAACAGCG (SEQ |
| | | ID NO: 13) |
| GAPDH (reverse); qPCR | Derived from (Kooreman et al., | ACCACCCTGTTGC |
| | 2017) | TGTAGCCAA (SEQ |
| | | ID NO: 14) |
| IL-15 (forward); qPCR | Derived from (Kooreman et al., | AACAGAAGCCAAC |
| | 2017) | TGGGTGAATG |
| | | (SEQ ID NO: 11) |
| IL-15 (reverse); qPCR | Derived from (Kooreman et al., | CTCCAAGAGAAAG |
| | 2017) | CACTTCATTGC |
| | | (SEQ ID NO: 12) |

Software and algorithms

| | | |
|---|---|---|
| FlowJo 10.7.1 software | FlowJo (Becton Dickinson) | https://www.flowjo. |
| | | com/ |
| sgRNA Designer, CRISPick | Broad institute | https://portals. |
| | | broadinstitute.org/ |
| | | gppx/crispick/public |

Other

| | | |
|---|---|---|
| 150 mm surface-treated tissue | Fisher Scientific | Cat# FB012925 |
| culture dishes | | |
| Steriflip-HV Sterile Centrifuge | Millipore Sigma | Cat# SE1M003M00 |
| tube top filter unit | | |
| Fisherbrand Disposable PES filter | Fisher Scientific | Cat# FB12566504 |
| Units (500 mL) | | |
| Olympus 384-well PCR plate | Genesee Scientific | Cat# 24-305 |
| 96-Well Cell Culture Plates (flat | Genesee Scientific | Cat# 25-109 |
| bottom) | | |
| 96-Well Cell Culture Plates (U | Genesee Scientific | Cat# 25-221 |
| bottom) | | |
| 48-Well Cell Culture Plates | Genesee Scientific | Cat# 25-109 |
| 24-Well Cell Culture Plates | Genesee Scientific | Cat# 25-107 |
| TC Treated Flasks, 25 mL, Vent | Genesee Scientific | Cat# 25-205 |
| (T-25) | | |
| 0.2 mL 8-Strip PCR Tubes, Natural | Genesee Scientific | Cat# 27-125 |
| 50 mL conical centrifuge tubes | Genesee Scientific | Cat# 28-106 |
| 15 ml conical centrifuge tubes | Genesee Scientific | Cat# 28-103 |
| FACS tubes (Falcon round-bottom | Fisher Scientific | Cat# 14-959AA |
| polypropylene tubes) | | |
| 14 mL Polypropylene round- | Corning life Sciences | Cat# 352059 |
| bottom tubes with caps | | |
| 1.7 mL Microtubes | Genesee Scientific | Cat# 22-281 |
| 0.6 mL Microtubes | Genesee Scientific | Cat# 24-272 |
| 1000 μl pipette tips | Genesee Scientific | Cat# 24-830 |
| 200 μl pipette tips | Genesee Scientific | Cat# 24-812 |
| 20 μl pipette tips | Genesee Scientific | Cat# 24-804 |
| 10 μl pipette tips | Genesee Scientific | Cat# 24-801 |
| 200 μl round gel loading tips, | Genesee Scientific | Cat# 24-113 |
| 0.57 mm | | |
| FACSymphony™ A5 flow | Becton Dickinson (BD) | n/a |
| cytometer | | |
| ProFlex PCR System | Thermo Fisher Scientific | n/a |
| NanoDrop™ One | Thermo Fisher Scientific | n/a |
| spectrophotometer | | |

TABLE 3
Growth media for NK-92 cell line

| Reagent | Final concentration |
|---|---|
| RPMI-1640 | N/A |
| FBS | 10% (v/v) |
| Penicillin-Streptomycin (100X) | 1% (v/v) |
| Sodium pyruvate (100 mM) | 1% (v/v) |
| Minimum Essential Media (MEM) | 1% (v/v) |
| non-essential amino acid (100X) | |
| GlutaMAX (100X) | 1% (v/v) |
| 2-Mercaptoethanol (50 mM) | 0.01% (v/v) |

All components were mixed and the media was filtered using sterile disposable vacuum filter with polyethersulfone (PES) membrane. This was the complete RPMI media, to grow NK-92 cells, supplement complete media with recombinant human (rh) IL-2 (100 U/mL). The media was stored at 4° C. for up to 1 month.

TABLE 4
Growth media for Lenti-X 293T cell line

| Reagent | Final concentration |
|---|---|
| DMEM | N/A |
| FBS | 10% (v/v) |
| Penicillin-Streptomycin (100X) | 1% (v/v) |
| Sodium pyruvate (100 mM) | 1% (v/v) |
| MEM non-essential amino acid | 1% (v/v) |
| (100X) | |
| GlutaMAX (100X) | 1% (v/v) |

All these components were mixed, and the media was filtered using sterile disposable vacuum filter with PES membrane. This was the complete DMEM media. The media was stored at 4° C. for up to 1 month.

TABLE 5
Growth media for K-562 cell line

| Reagent | Final concentration |
|---|---|
| RPMI-1640 | N/A |
| FBS | 10% (v/v) |
| Penicillin-Streptomycin (100X) | 1% (v/v) |
| Sodium pyruvate (100 mM) | 1% (v/v) |
| Minimum Essential Media (MEM) | 1% (v/v) |
| non-essential amino acid (100X) | |
| GlutaMAX (100X) | 1% (v/v) |
| 2-Mercaptoethanol (50 mM) | 0.01% (v/v) |

K-562 cell line was grown in complete RPMI media as described above. All the above-mentioned components were mixed and the media was filtered using sterile disposable vacuum filter with PES membrane. The media was stored at 4° C. for up to 1 month.

TABLE 6
Terrific Broth (TB) media (1 L)

| Component | Amount | |
|---|---|---|
| Terrific Broth powder | 47.6 | g |
| Glycerol | 4 | mL |
| Double distilled (dd) H₂O | Up to 1000 mL | |
| Ampicillin antibiotic | 100 | mg |

47.6 g TB powder was dissolved in 500 mL double distilled (dd) H₂O and 4 mL glycerol was added. After mixing, and the final media volume was made up to 1 L with ddH₂O and further autoclaved for 15 min at 121° C. The media was allowed to cool down to RT and ampicillin antibiotic was added (working concentration is 100 μg/mL). The media was stored at 4° C. for up to 1 month.

Flow Cytometry

Flow cytometry analysis was performed on BD FACSymphony™ A5 flow cytometer and data were analyzed using FlowJo 10.7.1 software.

TABLE 7

| Reagent | Final concentration |
|---|---|
| Anti-human IL-2Rγ (APC) | 1:100 |
| 4,6-diamidino-2-phenylindole | 1:5000 |
| (DAPI) (Stock 5 mg/mL) | |
| Fluorescence activated cell sorting | N/A |
| (FACS) Buffer (Ca²⁺ and Mg²⁺ free | |
| DPBS + 2% FBS + 0.09% sodium | |
| azide) | |

TABLE 8
Fluorescence activated cell sorting (FACS) Buffer

| Component | Final concentration | Amount | |
|---|---|---|---|
| Ca²⁺ and Mg²⁺ free DPBS | N/A | 490 | mL |
| FBS | 2% (v/v) | 10 | mL |
| Sodium azide | 0.09% (w/v) | 450 | mg |

The buffer was stored at 4° C. for up to 6 months.

sgRNAs each with a 5′ overhang “CACCG” were forwarded and sgRNAs each with 5′ “AAAC” were reversed and 3′ “C” overhangs were ordered from integrated DNA technologies (IDT). Refer to the section titled “oligo design for sgRNA production” for sgRNA design details. sgRNA sequences that were used included the nucleic acid sequences of SEQ ID NO:3, 4, 5, 6, 7, 8, 9, 10, 15, 16, 17, and 18. Primers used in qPCR include SEQ ID NO:11, 12, 13, 14 and 15.

Lentivirus production was about 5 days

On day 1, a 150 mm culture dish was coated with ˜3-5 mL of poly-L lysine solution (0.01%) by gently swirling to ensure even coating of the plate surface. The solution was aspirated and the coated plate was allowed to dry for 15 min at RT. The cell culture media was aspirated from Lenti-X 293T HEK cells grown in a 150 mm culture dish, these cells were washed with PBS, 2 mL of 1× Trypsin solution was added to the plate. The plate was gently swirled to evenly cover the whole surface area and incubated it for 1 min at 37° C. The cells were detached by gently tapping the plate and the Trypsin enzyme is inactivated by adding 10 mL of complete DMEM media. The cells were spinned down at 350×g for 5 min. The cells were resuspended in 10 mL of complete DMEM media, the cells were counted, and 7-10×10⁶ Lenti-X 293T HEK cells were seeded on the poly-L lysine-coated 150 mm plate. The total cell culture media volume was made up to 20 mL by adding complete DMEM media, the plate was gently swirled so that cells were uniformly seeded and incubated them at 37° C.

On day 2, two 15 mL tubes were taken, 4 mL of plain DMEM media was added (media without FBS and PS and contained only 1× GlutaMAX (glutamine), non-essential amino acids and sodium pyruvate in each tube. In the first tube, 20 μg of the plasmid was added containing the gene of interest (here, Lenti-Cas9 EGFP or dCas9-VP64-GFP or dCas9-KRAB-GFP), 20 μg of packaging plasmid (psPAX2), and 5 μg of envelope plasmid (pMD.2G). In the second tube, 80 μL of lipofectamine 2000 was added. Both tubes at RT for 5 min was incubated.

It was important to use plain DMEM i.e. serum-free DMEM, as serum inhibits transfection.

Lipofectamine-containing media was added with media containing the lentiviral vectors by gentle swirling or inverting, and the mix for 20 min at RT was incubated. After incubation, the culture media was aspirated from Lenti-X 293T HEK cells and the lipofectamine and plasmid mix were added to these cells in a dropwise fashion over the entire plate. The total media volume was made up to 15 mL by including additional 7 mL of plain DMEM to Lenti-X293T HEK cells. The plate was gently swirled and incubated at 37° C. for 3 h. After incubation, 15 mL complete FBS-containing DMEM media was added to the plate, the total media volume was made to 30 mL. The media was incubated overnight (12-16 h) at 37° C.

On day 3, the 30 mL of media was aspirated from culture dish and 20 mL of fresh complete DMEM media was added containing 400 μL of 500 mM sodium butyrate. Then, the media was incubated for 5 h at 37° C. to enhance the lentiviral production. After sodium butyrate induction, the media was discarded, the cells were replenished with 16 mL fresh complete DMEM media and incubated at 37° C. for 24 h.

On day 4, the media containing lentivirus was filtered using a sterile disposable vacuum filter unit (0.45 μm). Alternatives: Clarification of media might also be done with centrifugation (400×g for 5 min). Lentivirus was concentrated by adding 4 mL of lenti-X concentrator to 16 mL of viral supernatant, mixed well by pipetting, and incubated overnight (12-16 h) at 4° C.

On day 5, the mix at 1500×g for 45 min, 4° C. was centrifuged. After centrifugation, an off-white pellet will have formed at the bottom of centrifugation tube, without disturbing the pellet, the supernatant was aspirated by suction using a glass Pasteur pipette. Finally, the off-white pellet was resuspended in 1 mL of complete RPMI media.

Designing and cloning single guide RNAs (sgRNAs) for CRISPR-mediated gene editing and transcriptional modulation (about ˜2 days)

Oligo design for sgRNA production. Go to https://portals.broadinstitute.org/gppx/crispick/public Broad institute web portal (Doench et al., 2016). The references genome (here, Human GRCh37) were selected and then the mechanism of CRISPR editing (CRISPRko or CRISPRa or CRISPRi) was also selected. Enzyme selected was SpyoCas9. Quick look up in the target section was selected and the target gene I.D. (e.g., IL2RG for IL2R) was entered. The values in CRISPICK Quota box was entered (this value represented how many sgRNA per gene will be designed) and submitted. The results were downloaded by selecting picking result file (tab-limited text file). The file in excel was opened, this file contained the summary statistics for on- and off-target scores by pick order. The guide RNA was chosen sequence-based on pick order rank (minimum 2-5 sgRNAs per gene was chosen). Also the orientation of the sgRNA (sense or antisense) was checked. The reverse complement of the sgRNA sequence was designed. The following overhang to the sgRNA sequence was added which would facilitate its ligation to linearized ipUSEPR plasmid: Forward oligo: 5′ CACCG . . . guide RNA 3′, Reverse oligo:5′ AAAC . . . guide RNA . . . C 3′.

The oligos from Integrated DNA Technologies (IDT) Inc. was ordered.

Annealing. The oligo vial was thawed and spinned at 300×g for 2 min at RT and the oligos in nuclease-free water was reconstituted to make a 100 μM stock (the amount of water added into the oligo vial to make the 100 μM stock=10× number of nanomoles of oligo).

10 μl of the forward oligo (100 μM)+10 μl of the reverse oligo was mixed in a PCR tube and spinned down at 300×g for 2 min. A thermocycler was used to anneal forward and reverse oligos: 95° C. 10 min×1 cycle, Temperature was ramped down from 95° C. to 25° C., decrease rate=0.5° C./1 min×140 cycles (total time=140 min), held at 4° C. 1 μl of annealed oligo was taken from step 18, added into 2 mL of ddH₂O to made up a final dilution of 1/2000. These diluted oligos would be used as the insert for ligation.

Linearizing ipUSEPR plasmid for ligation (about 1.5 days)

Digestion of ipUSEPR plasmid. The digestion mixture was prepared in 0.6 mL microtubes as follows:

TABLE 9

| Reagent | Volume/Conc |
|---|---|
| ipUSEPR plasmid | 40 μg |
| Cutsmart 10X (Cat# B7204S) | 40 μl |
| BsmbI (Cat# R0739S) | 16 μl |
| Rnase free water | Fill up to 400 μl |

The mixture overnight (12-16 h) was incubated at 55° C. in a heat block. The linearized plasmid was isolated by electrophoresis (0.8% agarose gel, 120 V for 2 h) followed by gel extraction. 80 μl of 6× loading dye was added into 400 μl BsmbI digestion and 50 μl was loaded in each well (Marker: NEB #N0552S, Gel loading dye: NEB #B7025S). Gel extraction (GeneJET Gel Extraction kit #K0691): Empty Eppendorf tubes were weighed and the expected band was clipped from gel into Eppendorf tubes. The linearized ipUSEPR plasmid was purified as per manufacturer's instructions and after elution, DNA concentration was measured using a NanoDrop™ One spectrophotometer and the linearized plasmid at −20° C. was stored.

Ligation (Timing: ˜14 h)

Ligation mixture was prepared in 0.2 mL PCR tubes as follows:

TABLE 10

| Reagents | Volume | |
|---|---|---|
| Linearized ipUSEPR plasmid (60-100 ng/μl) | 1.5 | μl |
| 10X T4 ligase buffer (NEB # B0202S) | 0.6 | μl |
| Standard T4 ligase (NEB # B0202S) | 0.3 | μl |
| Master Mix (all above three products) | 2.4 | μl |
| 1/2000 diluted oligo (remember adding H₂O control) | 3.6 | μl |
| Total | 6 | μl |

A separate control tube was prepared which contained 3.6 μl of H₂O instead of the diluted oligo.

Ligation was performed using a thermocycler by following the conditions: 25° C. 1 h×1 cycle, 16° C. 12 h×1 cycle, held at 4° C. Ligation product was stored at −20° C. until transformation.

Transformation (NEB-5alpha competent E. coli #C2987U) (Timing: ˜18 h)

NEB 5-alpha competent E. coli cells were thawed on ice (50 μl/tube), and Ampicillin plates were brought to RT. 10 μl of competent cells were taken and mixed with 6 μl of the ligated product in a 0.6 mL microtube and then, incubated on ice for 30 min. Heat shock was provided at 42° C. for 30 s in a heat block and was put back on ice for 5 min. 180 μl of SOC medium was added and the bacteria was grown at 37° C. in a shaker incubator for 1 h. All the medium was spreaded onto the Ampicillin plate evenly and the plates were incubated upside down at 37° C. After 12-16 h, the plates at 4° C. was stored until miniprep.

Miniprep (GeneJET Plasmid Miniprep Kit #K0502) (Timing: ˜18 h)

4 mL TB was prepared with Ampicillin in a 14 mL tube, a single colony was picked with the help of a pipette tip from the bacterial plate into each tube, and shaken overnight (12-16 h) at 37° C. 1.8 mL bacteria was taken into a 2 mL tube, and centrifuged at 11000×g for 30 s at RT to pellet down the bacteria. The miniprep was prepared of plasmid DNA as per manufacturer's instructions. In the final elution step, 50 μl of elution buffer was used to elute the plasmid DNA from the column and concentration of the plasmid was measured and stored at −20° C. 8 μl of 100-200 ng/μl DNA and 5 μl of 5 μM hU6 primer: “GAGGGCCTATTTCCCATGAT” (SEQ ID NO:19) per sample were sent to Eton Biosciences for sequencing to confirm the sgRNA sequence.

Maxiprep (NucleoBond Xtra Maxi Plus EF #740426.50) (Timing: ˜1.5 Days)

4 mL of TB medium was taken in a 14 mL tube and inoculated with a single colony picked from ampicillin plate. Incubated at 37° C. in a shaker incubator at 250 RPM for 5-6 h. ˜1 mL of the inoculated media was added to 250 mL of TB media and the culture was grown overnight (12-16 h) at 37° C. in a shaker incubator at 250 RPM. Bacterial cells were harvested by centrifuging at 6000×g for 15 min at 4° C. Then the supernatant was discarded. The plasmid DNA was isolated as per manufacturer's instructions. In the final elution step, 400 l of elution buffer was used to elute plasmid DNA from NucleoBond finalizer (provided in the kit). Plasmid DNA concentration was measured and stored at −20° C.

Significance of Genetically Manipulating Natural Killer Cells Using CRISPR

Natural killer (NK) cells were innate immune cells that protected against viral infections (Strauss-Albee and Blish, 2016) and development of cancers (Cerwenka and Lanier, 2001). NK cell-mediated immune surveillance was defective in infections and cancers for various reasons including NK cell exhaustion, defective homeostasis, and impaired NK cytotoxicity (Duault et al., 2021, Wilk et al., 2021). Overall goal was to develop therapeutic NK cells which were functional and evaded exhaustion through CRISPR-based genetic manipulation. To this end, NK-92 cells were manipulated, one of the platforms for NK therapies in clinical trials (Klingemann et al., 2016), using CRISPRko/Cas9 for knockout (Jinek et al., 2012), CRISPRi/dCas9-KRAB for transcriptional repression (Gilbert et al., 2013), and CRISPRa/dCas9-VP64 for transcriptional activation (Chavez et al., 2015) of genes that promoted NK cytotoxicity and reduced NK exhaustion.

Below presents the detailed protocols for generation of each of these multifunctional CRISPR-based NK cell platforms.

Generating Cas9⁺ NK-92 cells (CRISPRko NK-92 platform) (Timing: ˜7 weeks)

NK-92 cells were counted, ˜0.5-1.5×10⁶ cells were resuspended in 500 μl of complete RPMI media, and 500 μl of concentrated Cas9-EGFP lentivirus (concentrated from 16 mL of lentiviral supernatant to 1 mL using lenti-X concentrator) was added. Now the total volume would be 1 mL; to this, polybrene (10 μg/mL) and rhIL-2 (100 U/mL) were added. These cells were seeded in a 24-well plate (1.5×10⁶/mL/well) and incubated at 37° C. for 48 h.

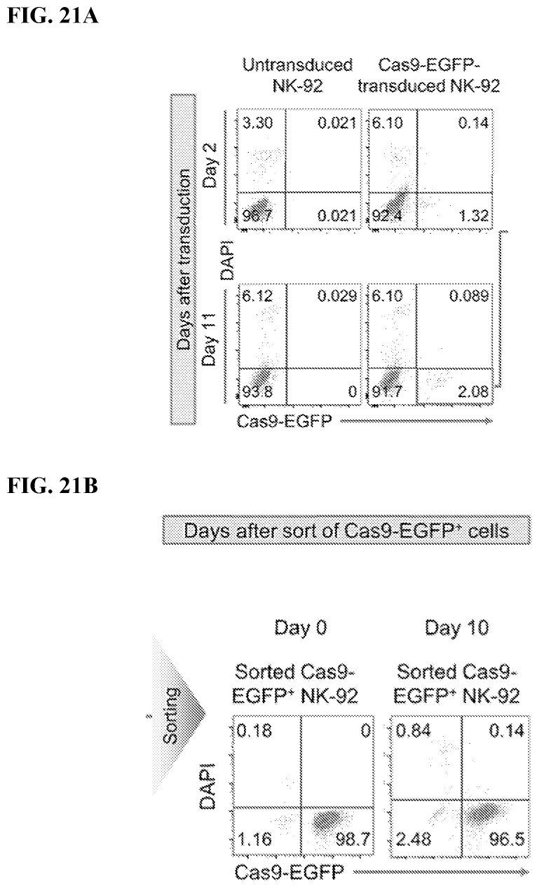

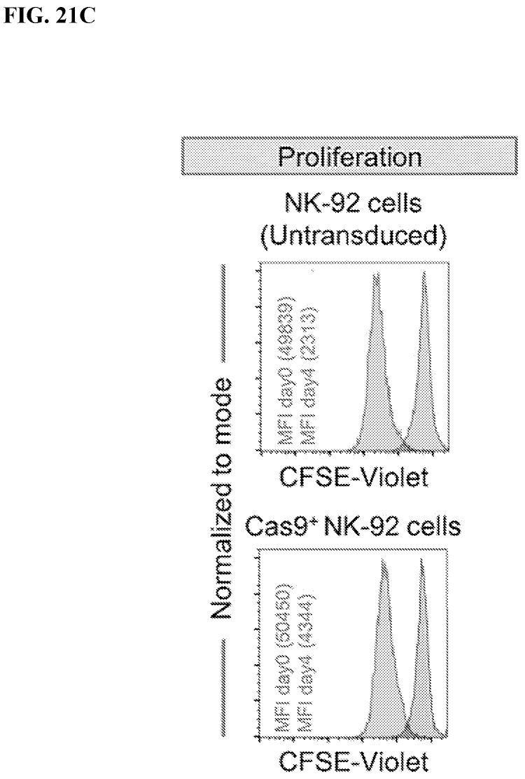

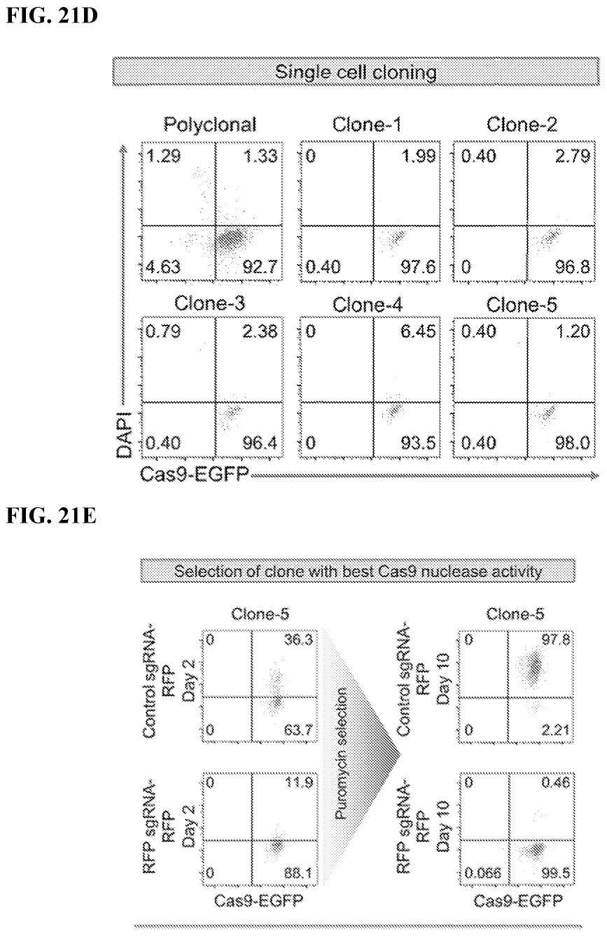

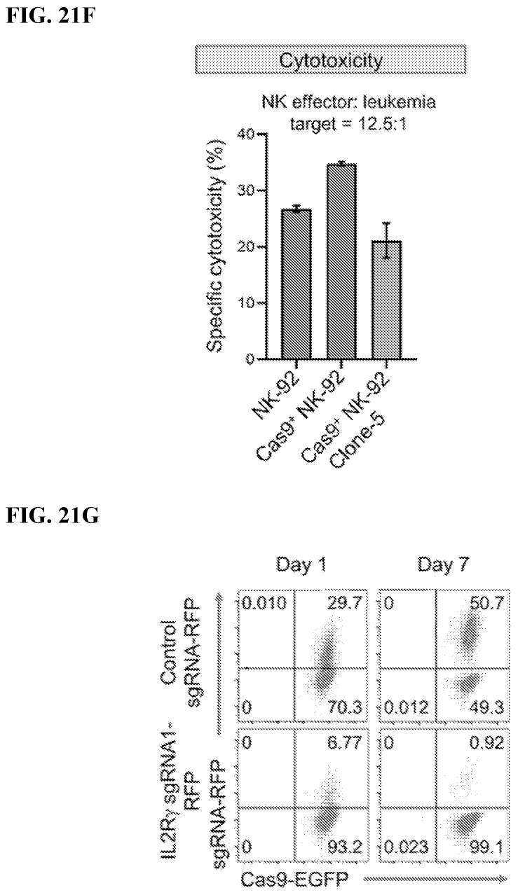

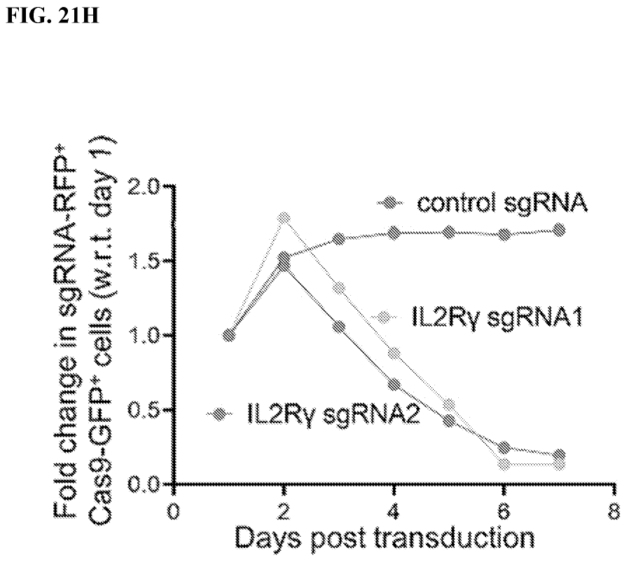

On day 2, the cells were washed with complete RPMI media, and the NK-92 cells were replenished with NK cell media (RPMI media+rhIL-2). A small aliquot of the transduced cells (˜100 μl) were pipetted into a 1.7 mL tube to measure percentages of EGFP⁺ Cas9-transduced NK-92 cells by flow cytometry (FIG. 21A upper panel). Because all NK cells including NK-92 were generally difficult to transduce, a transduction efficiency of only 1.32% was obtained after transducing NK-92 cells with Cas9 (FIG. 21A, upper panel).

The Cas9-transduced NK-92 cells were expanded for the next 8-10 days by changing the NK cell media and the cells were re-planted on every third day. Then, a small aliquot of cells (˜0.5-1×10⁶) were pipetted and stained with DAPI, and the percentages of DAPI⁻Cas9-EGFP⁺ viable cells (FIG. 21B, lower panel) were checked. This step was to ensure that transduction with Cas9 does not reduce the viability of NK-92 cells. It was observed that NK cell viability was similar in untransduced and Cas9-transduced NK-92 cells (FIG. 21A).

If the transduction efficiency was low, then it would be difficult to sort the transduced cells using FACS. In case of transduction efficiencies lower than 1%, cells for 1-2 weeks were expanded and transduced them again with an additional 500 μl of concentrated virus to increase the frequencies of transduced cells.

After expansion, stain cells were stained with DAPI and sort DAPI⁻EGFP⁺ cells. Check the post-sort purity by flow cytometry (FIG. 21B, Day 0). The post-sort purity by flow cytometry was checked (FIG. 21B, Day 0). The sorted cells were expanded as described below. The sorted cells were counted and ˜0.5-1×10⁵ cells/well were seeded in a 96-well flat bottom plate. These cells were expanded in NK media at 37° C. When cells became confluent (media color changes to light yellow), the cells (300×g at RT for 5 min) were spinned down and the media was aspirated. The cells were resuspended in fresh 500 μl NK cell media and cultured them in a 48 well plate at 37° C. It was held until the cells became confluent, media was spinned down and aspirated and transferred to T-25 flask containing 5 mL of fresh NK cell media. After expansion of NK-92 cells in a T-25 flask for 3 days, once again flow cytometry was conducted to ensure that only EGFP⁺ cells were expanding (FIG. 21B, Day 10). Please note that cells from a single well of 96-well plate would be transferred to single well of 48-well plate and then to a T-25 flask.

The proliferation potential of transduced NK-92 cells was compared with that of untransduced NK-92 cells to ensure that transduction with Cas9 does not reduce NK-92 cell proliferation: a vial of CFSE-violet dye (Thermo Scientific #C34557) was reconstituted in DMSO and 20 μl of DMSO was added in the vial to make a 5 mM stock. The untransduced and Cas9-transduced NK-92 cells (300×g for 5 min at RT) were spinned down. The culture media was aspirated and the cells in PBS was resuspended. The cell count was adjusted to 1×10⁶ NK-92 cells/mL in a 15 mL tube. 0.5 l/mL of CFSE-violet dye was added and incubated these cells for 20 min at 37° C. in the dark. After incubation, at least 5 times the original staining volume of complete RPMI media was added and incubated for 5 min at 37° C. The cells (300×g for 5 min) was centrifuged, the media was discarded, and the cells was washed again with complete RPMI media. The cell count was adjusted to 1×10⁶ NK-92 cells/mL in NK cell media and 200 μl was seeded in 96-well flat-bottom plate (0.2×10⁶ cells/well). The cells were incubated for 96 h at 37° C. and then analyzed using flow cytometry for CFSE dilution (FIG. 21C). The data was analyzed using FlowJo software and the median fluorescence intensity (MFI) values was noted down for CFSE and the proliferation score (PS) was calculated using formula PS=Log₂ (MFI_{CFSE day 0}/MFI_{CFSE day 4}) (Ahlen et al., 2009). A less than 2-fold difference was found in the PS of untransduced NK-92 cells (PS=4.4) and Cas9⁺ NK-92 cells (PS=3.5), suggesting that transduction with Cas9 did not abrogate or markedly impair NK-92 cell proliferation.

Single cell isolation using limiting dilution technique: Cas9-EGFP⁺ NK-92 cells were counted using a hemocytometer and the cell count to 5×10³/mL was adjusted. The final cell counts seeded a 96-well plate were found to be 5 cells/mL, and to achieve that count, 50 μl of 5×10³/mL solution with 49.95 mL of NK cell media. 100 μl of 5 cells/mL solution was transferred to each well of 96-well round-bottom plates and incubated at 37° C. These cells were carefully monitored over a period of two weeks. When single-cell clumps had grown in size, these cells from 96-well plates were removed using a 1 mL pipette, washed, and transferred to 48-well culture plate in NK cell media (500 μl/well). After three days of culture in 48-well plate, percentages of Cas9-EGFP⁺ NK-92 cells were analyzed in each clone using flow cytometry (FIG. 21D) and the cells in a T-25 flask was cultured in NK cell media.

The cell count was accurately adjusted to 5 cells/mL and 100 μl was seeded of this mix per well was most important step in the single NK cell expansion protocol. This minimized the likelihood of seeding more than 1 cell/well. This step also prevented the expansion of untransduced EGFP⁻ NK-92 cells in the culture.

Alternative method for single cell isolation: Flow cytometry-based sorting of single cell clones. However, sorting was not preferred because it was stressful for sensitive cells such as, human NK cells.

Cytotoxic Cas9⁺ NK-92 single cell clones was selected for CRISPRko editing (Timing: ˜2 weeks)

Cas9⁺ NK-92 cells single cell clones were first evaluated for CRISPR editing efficiency by using a puromycin-tagged sgRNA that targets and knocks out the RFP present in the ipUSEPR sgRNA cloning plasmid, as described below:

Single cell clones were transduced: each single cell clone was resuspended (here, clone-1 to -5, FIG. 21D) in 1000 μl complete RPMI media (1×10⁶ cells/clone) in 1.7 mL microtubes. Single cell suspension was divided from each clone into two parts and transferred into 1.7 mL microtubes. To one portion, 500 μl control sgRNA lentivirus was added and to other half 500 μl lentivirus harboring sgRNA was added targeting RFP of ipUSEPR plasmid.

Now the total volume in each tube would be 1 mL, to this, polybrene (10 μg/mL) was added and rhIL-2 (100 U/mL). These cells were seeded in 24-well plate (0.5×10⁶/well) and incubated at 37° C. for 48 h.

On day 2, frequencies of sgRNA-transduced RFP⁺ cells were analyzed by flow cytometry. The remaining cells was resuspended in 2 mL complete RPMI media containing rhIL-2 (100 U/mL) and puromycin (FIG. 21E, left panels).

After selecting transduced cells using puromycin for ˜10 days, it was found that clone-5 showed the best CRISPR editing efficiency with percentages of RFP⁺ cells reducing from 11.9% at day 2 to 0.46% at day 10 after puromycin selection (FIG. 21E). Note that procedures for design and cloning of sgRNAs and production of sgRNA lentiviruses have been described previously.

Before knocking out genes of interest in Cas9-transduced NK-92 single cell clones, one must confirm that constitutive expression of Cas9 does not impair the normal function (i.e. the cytotoxicity) of NK-92 cells.

Comparison among the cytotoxic function of untransduced, bulk (polyclonal), and single cell Cas9-transduced NK-92 cells using a flow cytometry-based NK cytotoxicity assay was performed. In this assay, the ability of untransduced and Cas9⁺ NK-92 cells were measured to kill the standard NK-sensitive target human erythroleukemia cell line, K-562. Among the Cas9⁺ NK-92 single cell clones, only clone-5 was utilized for the NK cytotoxicity/functionality assay because it was found to have the highest gene editing efficiency (FIG. 21E). Below presents the detailed procedure to conduct the NK cytotoxicity assay:

To distinguish NK-92 effector cells from target K-562 cells, label K-562 cells with CFSE using the procedure below: a vial of CFSE-violet dye (Thermo Scientific #C34557) was reconstituted by adding 20 μl DMSO in the vial to make a 5 mM stock. K-562 cells (300×g for 5 min at RT) was spinned down, the cell pellet was resuspended in PBS in a 15 mL tube and the cell count was adjusted to 1×10⁶ K-562 cells/mL. 0.5 l/mL of CFSE-violet dye was added and these cells were incubated for 20 min at 37° C. in dark. After incubation, at least 5 times the original staining volume was added of complete RPMI-1640 media (RPMI-1640+10% FBS) and incubated for 5 min at 37° C. The cells at 300×g for 5 min at RT was centrifuged, the media was discarded, and the cells were washed again with 5 mL of complete RPMI media before using them in cytotoxicity assays. NK-92 effector and K-562 target cells were counted. These cells were pipette mixed at different effector to target ratios in a U bottom 96-well plate in 200 μl complete RPMI media. The plate at 300×g for 5 min at RT was centrifuged and incubated for 5 h at 37° C. A control group was included without a miss that contained only CFSE labeled K-562 target cells to measure spontaneous cell death of the target in the absence of NK effector cells.

After incubation, 3 μl of 7-AAD (50 μg/mL) was added in each well and mixed by pipetting. The plate was incubated for 10 min at RT followed by transferring of cells into FACS tubes and the cells were analyzed by flow cytometry (FIG. 21F). The specific cytotoxicity was calculated using the formula: [(7-AAD⁺ target-cell frequency in coculture with effector cells (7-AAD⁺ target-cell frequency alone)/(100−7-AAD⁺ target-cell frequency alone)]×100.

Gene Knockout Validation in Cas9⁺ NK-92 Cells (Timing: ˜10 Days)

As NK-92 cells were dependent on the cytokines IL-2 and IL-15 for their survival, knockout of the IL-2R, to which these cytokines bound, should led to the death of NK-92 cells (Carson et al., 1997, Tornroos et al., 2019). Therefore, it knocked out a required signaling component of the IL2R, the IL2Rγ chain (Waickman et al., 2016) to validate the successful generation of Cas9⁺ NK-92 cells. The procedures for designing and cloning IL2Rγ sgRNA, and that for producing IL2Rγ sgRNA lentiviruses have been described earlier.

To validate IL2Rγ gene knockout in Cas9⁺ NK-92 cells: Cas9-EGFP⁺ NK-92 cells were counted, 1×10⁶ cells were resuspended in 1000 μl of complete RPMI media. 500 μl of concentrated lentivirus was added for control sgRNA or sgRNA targeting IL2Rγ chain. Now the total volume in each tube would be 1 mL. To these tubes, polybrene (10 μg/mL) was added and rhIL-2 (100 U/mL). These cells were seeded in a 24-well plate (0.5×10⁶ cells/well) and incubated at 37° C. for 48 h. Material and equipment section was referred for sequences of control or IL-2RG sgRNA used for CRISPRko.

On day 2, the cells were washed with complete RPMI media, the cells were resuspended in 2 mL of media containing rhIL-2 (100 U/mL) and seeded them in 24-well plate. Before seeding, a small aliquot of cells was taken to determine the frequencies of transduced cells by flow cytometry. On day 2 after sgRNA transduction, the levels of IL-2R was monitored on transduced NK-92 cells by flow cytometry (FIG. 21G-21H). Successful IL2R knockout was confirmed by the reduction in percentages of IL2Rγ sgRNA-RFP⁺ Cas9-EGFP⁺ cells over time as these cells failed to respond to IL-2 present in the culture medium. However, frequencies of control sgRNA-RFP⁺ Cas9-EGFP⁺ NK-92 cells did not reduce with time (FIG. 21G-21H), confirming that the transduction procedure itself doesn't kill NK cells.

Generating dCas9-KRAB⁺ NK-92 Cells (CRISPRi NK-92 Platform) (Timing: ˜7 Weeks)

The CRISPRi system employed a catalytically dead Cas9 (dCas9) without endonuclease activity fused to the KRAB transcriptional repressor (Qi et al., 2013). Specific sgRNAs allowed the recruitment and binding of the dCas9-KRAB transcriptional repressor to target genes (Gilbert et al., 2013). The dCas9-KRAB-mediated transcriptional repression of genes was an alternative approach to the Cas9-mediated CRISPRko system. The dCas9-KRAB system mitigated the risk of off-target effects and exogenous DNA integration caused by the active Cas9 nuclease in the CRISPRko system (Tycko et al., 2019, Hanlon et al., 2019, Saayman et al., 2016).

Below presents the detailed protocol for generating and validating NK-92 cells that constitutively expressed the dCas9-KRAB for CRISPRi-mediated transcriptional repression.

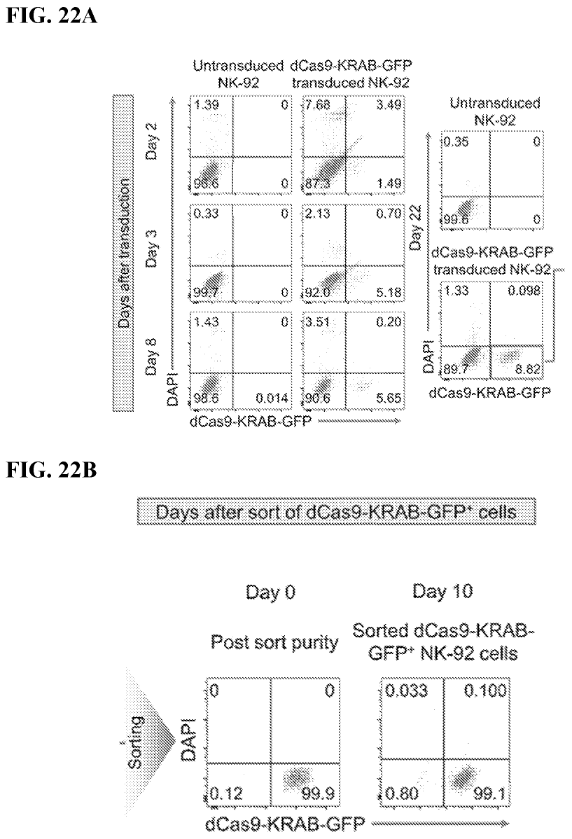

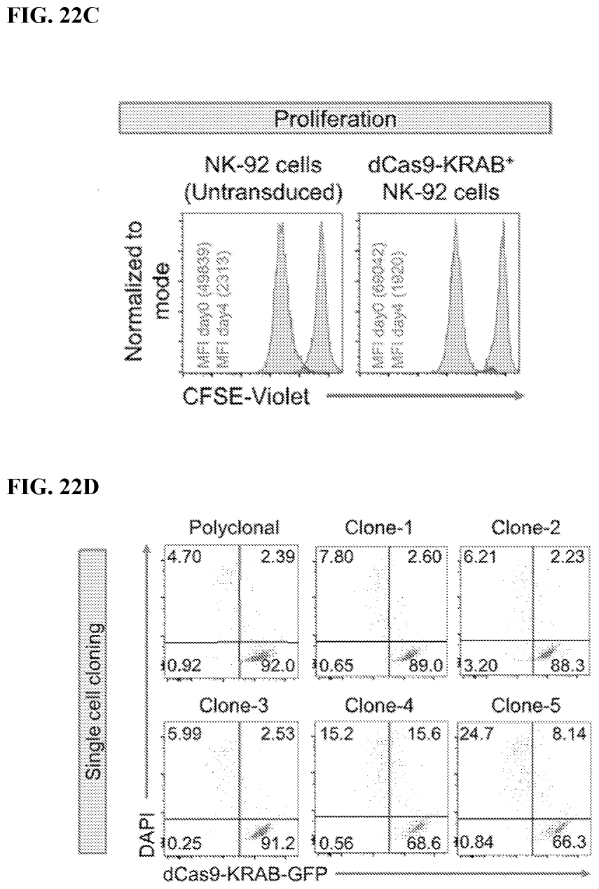

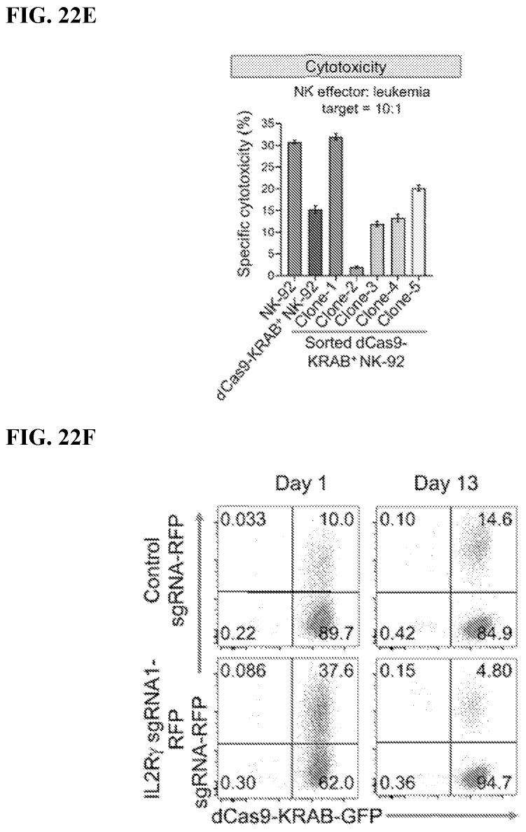

As Cas9⁺ NK-92 cells were generated, ˜0.5-1.5×10⁶ cells were resuspended in 500 μL complete RPMI media+500 μl concentrated dCas9-KRAB-GFP lentivirus+polybrene (10 μg/mL)+rhIL-2 (100 U/mL). These cells were seeded in 24-well plate and incubated 37° C. for 48 h. After incubation, washed and resuspended in complete RPMI media supplemented with rhIL-2. Percentages of dCas9-KRAB-GFP-transduced NK-92 cells was analyzed using flow cytometry. These cells were expanded to get enough cells for sorting with intermittent flow cytometry analysis of cells for GFP positivity (FIG. 22A).

The cells were stained with DAPI and sort DAPI⁻GFP⁺ cells, post sort purity was checked (FIG. 22B). Proliferation potential (FIG. 22C) was analyzed and the proliferation score (PS) was calculated as described previously (step 48) for Cas9⁺ NK-92 cells. It was found that PS of dCas9-KRAB⁺ NK-92 cells to be 5.4 which was less than 2-fold different from that of untransduced NK-92 cells (PS=4.4). The comparable PS of untransduced and dCas9-KRAB NK-92 cells suggested that constitutive expression of the dCas9-KRAB protein in NK-92 cells for CRISPRi does not abolish or markedly impair their natural proliferation. Single cell clones (FIG. 22D) were isolated using the limiting dilution technique as described previously.

Cytotoxic dCas9-KRAB NK-92 Single Cell Clones were Selected for CRISPRi Editing (Timing: ˜6 h)

Because the dCas9-KRAB system used a catalytically dead Cas9 without endonuclease activity, the sgRNA targeting RFP was not used and it was previously used to measure Cas9 editing efficiency. Instead, all dCas9-KRAB+ NK-92 single cell clones were screened that had expanded in culture for NK cytotoxicity by flow cytometry as described below. Using a flow cytometry-based NK cytotoxicity assay (as described in step numbers: 53-55), the ability of untransduced and dCas9-KRAB+ NK-92 cells were measured to kill the standard NK-sensitive target human erythroleukemia cell line, K-562, as described earlier. Among the Cas9-KRAB NK-92 single cell clones, only clone-1 showed the highest cytotoxic activity against K-562 cells (FIG. 22E). Therefore, only this clone was utilized for validation of the dCas9-KRAB system.

Transcriptional Repression Validation in dCas9-KRAB⁺ NK-92 Cells (Timing: 15 Days)

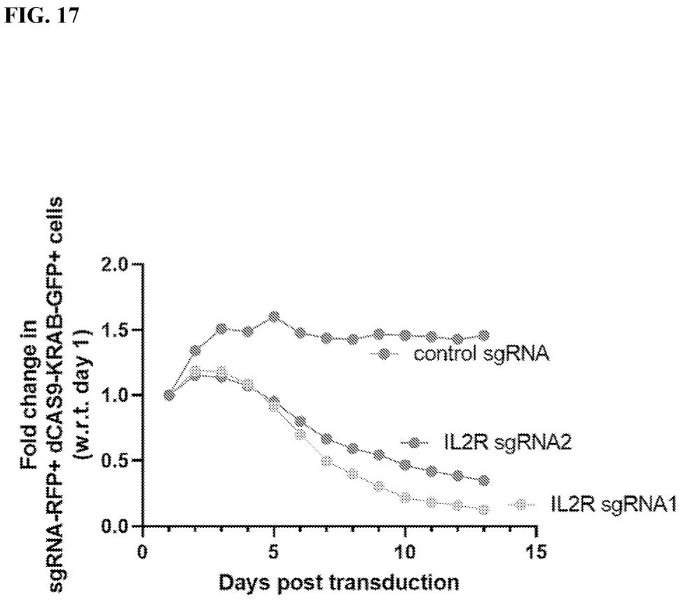

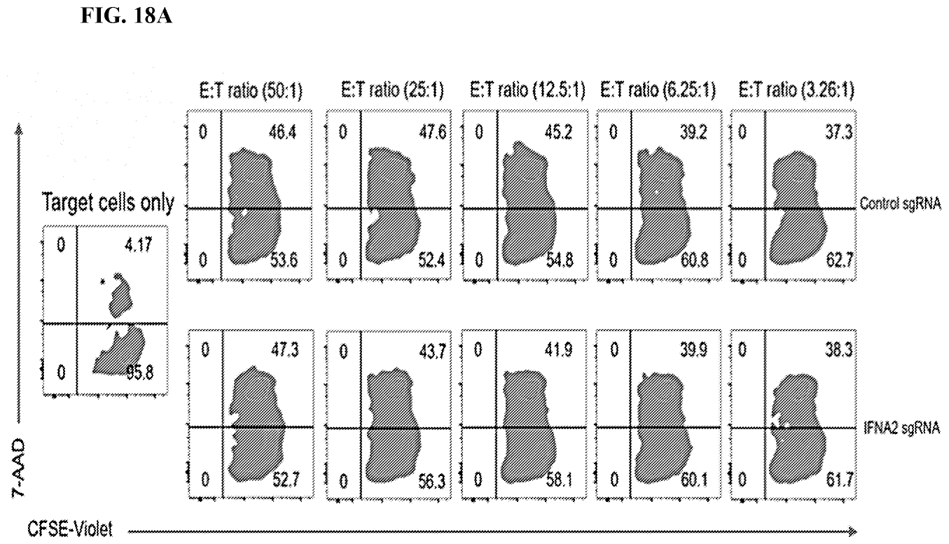

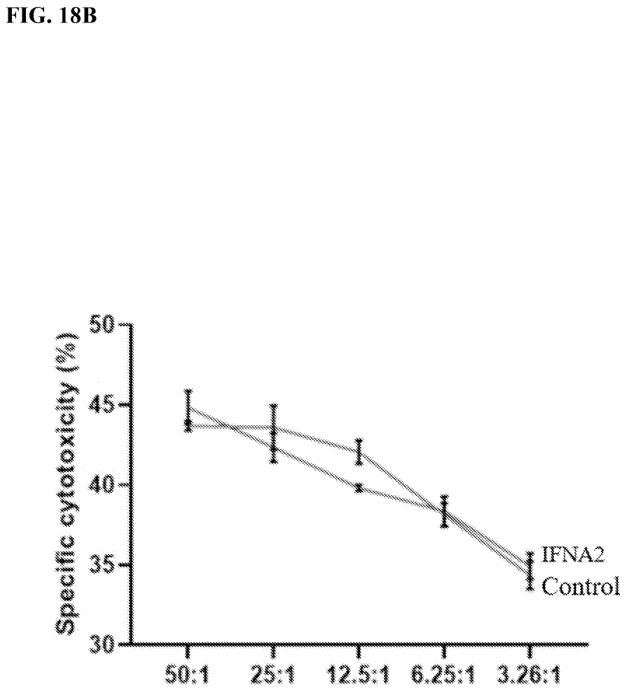

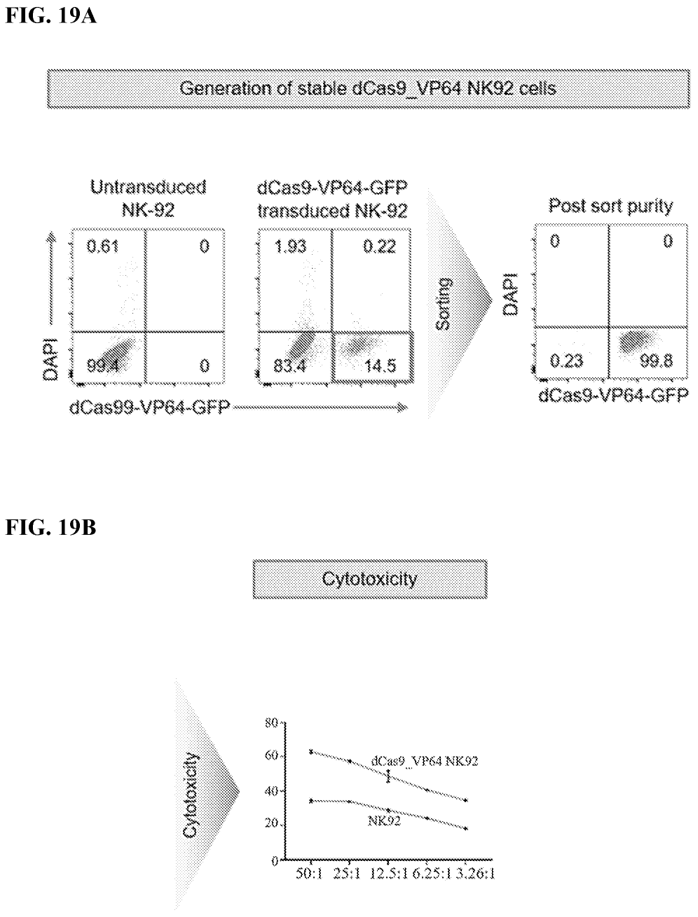

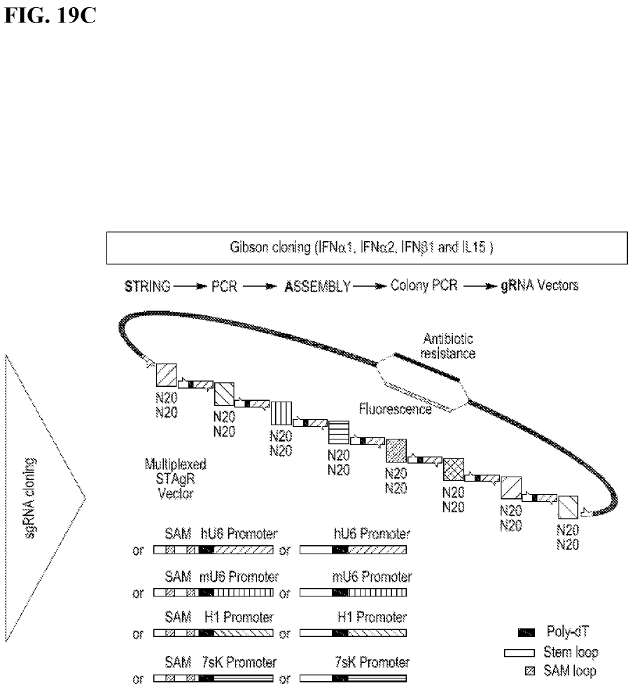

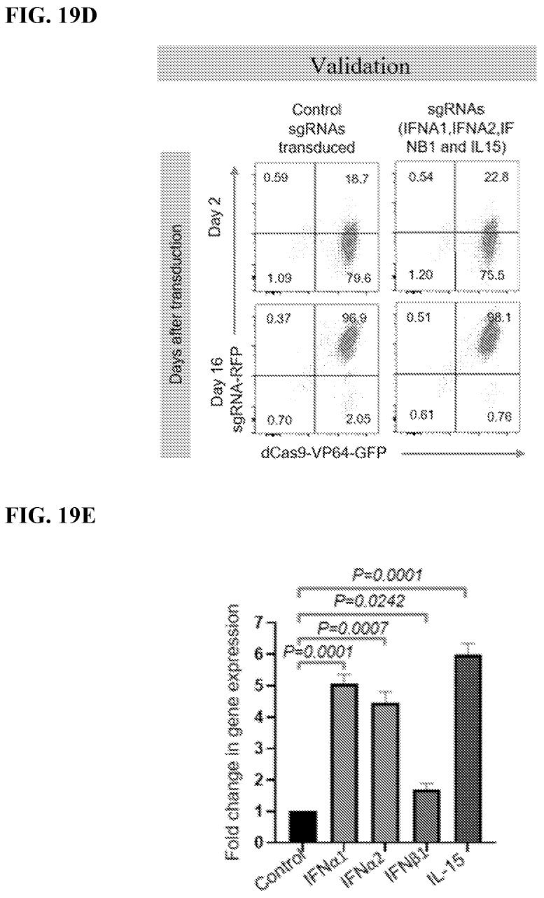

Transcriptional repression was validated in dCas9-KRAB in NK-92 cells using the same validation strategy as that described in Cas9⁺ NK-92 cells, i.e. blocking the expression of the IL2Rγ chain required for NK-92 cell proliferation and survival, and the effects were examined of this blockade on NK-92 cell turnover. The procedures for designing and cloning IL2Rγ sgRNA lentiviruses have been described earlier (steps 1-43). Also, the procedure for sgRNA lentiviral transduction of dCas9-KRAB+ NK-92 cells was identical to that outlined for Cas9⁺ NK-92 cells (step 44). Starting on day 2 after transduction, the levels of IL2Rγ chain was serially monitored on transduced NK-92 cells by flow cytometry (FIGS. 22F and 17). Successful IL2R knockout was confirmed by the reduction in percentages of IL2Rγ-sgRNA-RFP⁺ dCas9-KRAB-GFP⁺ cells over time as these cells failed to respond to IL-2 present in the culture medium.

dCas9-VP64⁺ NK-92 Cells Generated (CRISPRa NK-92 Platform) (Timing: ˜7 Weeks)

The CRISPRa system employed a catalytically dead Cas9 (dCas9) without endonuclease activity fused to the VP64 transcriptional activator (Qi et al., 2013). Specific sgRNAs allowed the recruitment and binding of the dCas9-VP64 transcriptional activator to target genes (Gilbert et al., 2013). Like the dCas9-KRAB system, the dCas9-VP64 system has reduced risk of off-target effects and exogenous DNA integration unlike the CRISPRko system (Tycko et al., 2019, Hanlon et al., 2019, Saayman et al., 2016). The CRISPRa approach to transcriptionally activate genes in human NK cells was superior to conventional lentiviral overexpression for two reasons: First, it was easier to introduce ˜20 nt single guide (sgRNAs) to simultaneously transcriptionally activate multiple genes by CRISPRa as opposed to sequentially transducing NK cells with large lentiviral overexpression vectors encoding the large open reading frame of each gene. Second, because transcript levels increased by ˜3-5-fold in CRISPRa as opposed to >20-fold in conventional overexpression systems, cellular toxicity caused by excessive production of proteins might be avoided in CRISPRa.

Below presents the detailed protocol for generating and validating NK-92 cells that constitutively expressed the dCas9-VP64 for CRISPRa-mediated transcriptional activation.

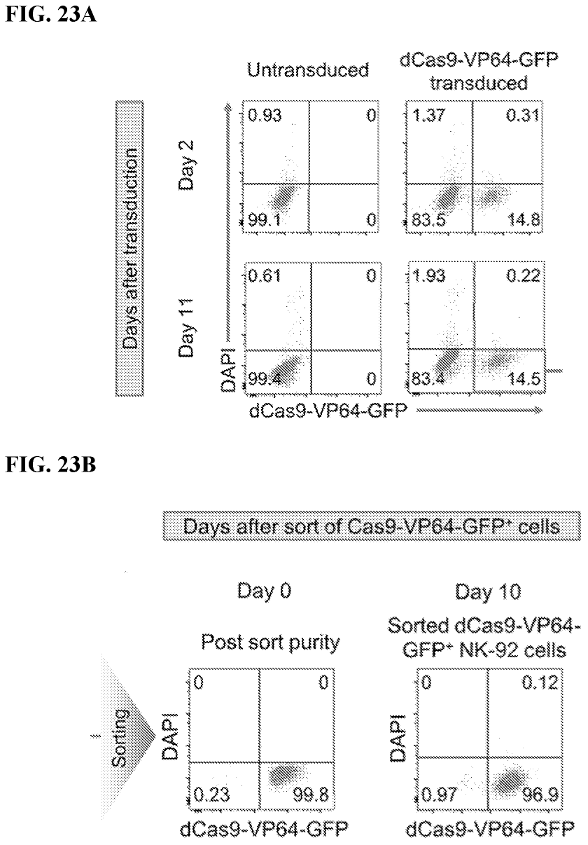

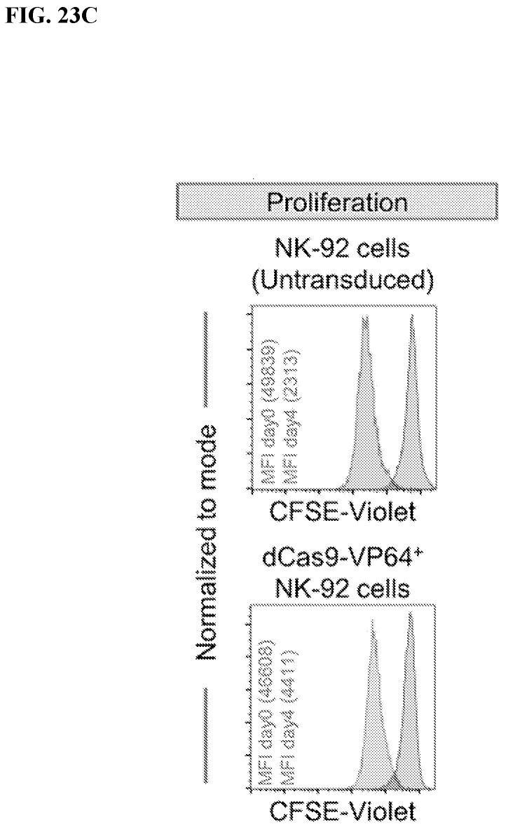

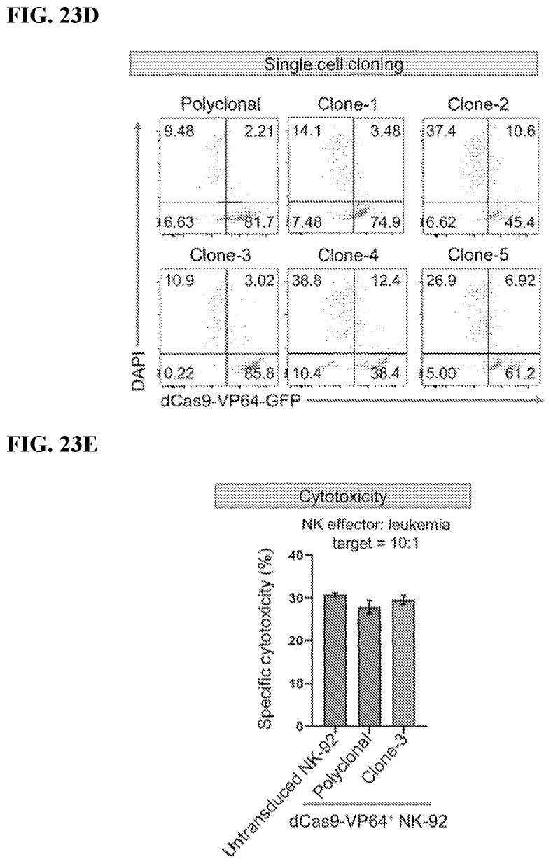

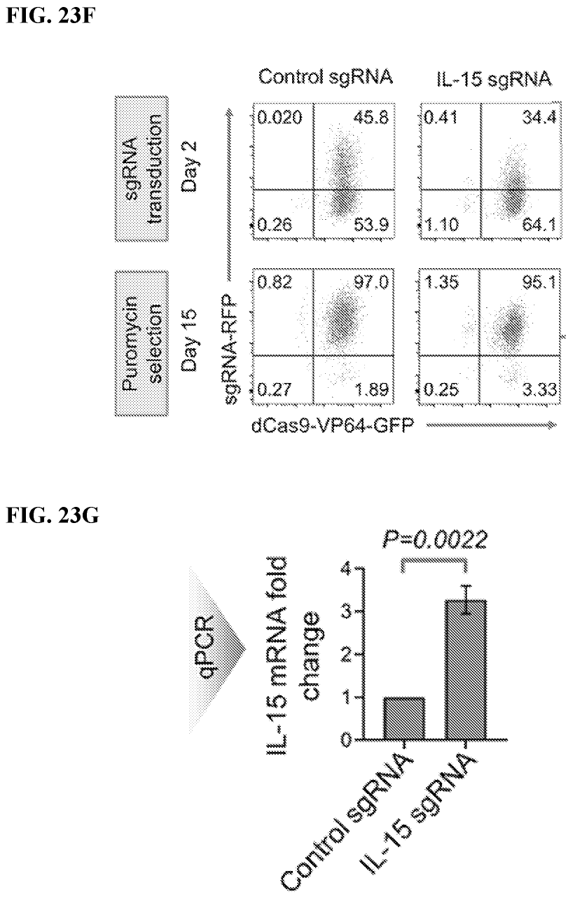

As Cas9⁺ and dCAS9-KRAB NK-92 cells was generated, NK-92 cells were counted, ˜0.5-1.5×10⁶ cells were resuspended in 500 μl of complete RPMI media in a 1.7 mL microtube, and 500 μl of concentrated dCas9-VP64-GFP lentivirus was added. Now the total volume would be 1 mL. To this, polybrene (10 μg/mL) and rhIL-2 (100 U/mL) were added. These cells were seeded in a 24-well plate and incubated at 37° C. for 48 h. On day 2, the cells were washed and replenished with complete RPMI media+rhIL2 (100 U/mL). A small aliquot of cells was taken to measure the percentages of GFP⁺ dCas9-VP64-transduced NK-92 cells using flow cytometry. It was found that the percentages of dCas9-VP64-transduced NK-92 cells were 14.8% at day 2 and 14.5% at day 11 after expansion (FIG. 23A).

The cells were stained with DAPI and DAPI GFP⁺ NK-92 cells were sorted by FACS. The post-sort purity was checked by flow cytometry. It was found that post-sort purities of dCas9-VP64-GFP⁺ NK-92 cells were 99.8% at day 0 and 96.9% at day 10 after expansion (FIG. 23B). The proliferation of dCas9-VP64⁺ NK-92 cells and that of untransduced NK-92 cells was compared (FIG. 23C) by measuring the proliferation score (PS) using the formula previously described (step 48) for Cas9⁺ and dCAS9-KRAB NK-92 cells. It was found that PS of dCas9-VP64⁺ NK-92 cells to be 3.4 which was less than 2-fold different from that of untransduced NK-92 cells (PS=4.4). The comparable PS of untransduced and dCas9-VP64⁺ NK-92 cells suggested that constitutive expression of the dCas9-VP64 protein in NK-92 cells for CRISPRa does not abolish or markedly impair their natural proliferation. The single cell clones were isolated of dCas9-VP64⁺ NK-92 cells (FIG. 23D) using the limiting dilution procedure previously described for Cas9⁺ and dCas9-KRAB NK-92 cells in FIG. 21D, 22D.

Cytotoxic dCas9-VP64⁺ NK-92 Single Cell Clones were Selected for CRISPRa Editing (Timing: ˜6 h)

Because the dCas9-VP64 system used catalytically dead Cas9 without endonuclease activity, the sgRNA targeting RFP was not used which was previously used to measure Cas9 editing efficiency. Instead, dCas9-VP64⁺ NK-92 single cell clones were directly screened that had expanded in culture for NK cytotoxicity by flow cytometry. Of note, all dCas9-VP64⁺NK-92 cells except clone-3 had contamination from GFP negative (dCas9-VP64⁻) NK-92 cells FIG. 23D). Therefore, the cytotoxicity assay was performed only using clone-3 of dCas9-VP64⁺ NK-92 cells (FIG. 23E). The procedure for performing cytotoxicity assays was similar to that described earlier (steps 53-55) for Cas9⁺ and dCas9-KRAB+ NK-92 cells.

Transcriptional Activation Validation in dCas9-VP64⁺ NK-92 Cells (Timing: ˜2-4 Weeks)

To validate dCas9-VP64⁺ NK-92 cells, the IL-15 gene was transcriptionally activated which was required for not just survival and proliferation but also the cytotoxic function of NK cells (Cooper et al., 2002, Zhang et al., 2018). Alternately, one could transcriptionally activated IL-2. The procedures for designing and cloning IL-15 sgRNA, and that for producing IL2Rγ sgRNA lentiviruses have been described earlier.

To validate transcriptional activation of IL-15 gene in dCas9-VP64⁺ NK-92 cells:

1×10⁶ dCas9-VP64-GFP⁺ NK-92 cells were resuspended in 1000 μl of complete RPMI media in 1.7 mL microtube. The cell suspension was divided into two parts and transfer to 1.7 mL microtubes. 500 μl of concentrated lentivirus was added for control sgRNA-RFP or IL-15 sgRNA-RFP targeting the IL-15 gene. Now the total volume in each tube would be 1 mL. To these tubes, polybrene (10 μg/mL) and rhIL-2 (100 U/mL) were added. These cells were seeded in a 24-well plate (0.5×10⁶ cells/well) and incubated at 37° C. for 48 h. Material and equipment section were referred for sequences of control sgRNA or IL-15 sgRNA used for CRISPRa. On day 2, before puromycin selection, a small aliquot of transduced cells was taken and the frequencies of dCas9-VP64-GFP⁺ sgRNA-RFP⁺ transduced cells were analyzed by flow cytometry (FIG. 23F, upper panel). The remaining cells in NK cell media was resuspended containing puromycin (1 μg/mL) and these cells were selected in puromycin for ˜2-4 weeks until frequencies of dCas9-VP64-GFP⁺ sgRNA-RFP⁺ cells in the control sgRNA and IL-15 sgRNA groups reaches >95% (FIG. 23F, lower panel).

The time period of puromycin selection might be prolonged and/or selection might be difficult if the gene modulated negatively impacts NK cell turnover. For such sgRNAs, flow-based sorting of dCas9-VP64-GFP⁺ sgRNA-RFP⁺ NK cells were recommended. RNA isolation (NucleoSpin RNA purification kit #740955.250) was proceeded, cDNA synthesis (SuperScript IV First Strand Synthesis System #18091050) and the mRNA expression was quantitated of IL-15 in cells transduced with control sgRNA or IL-15 sgRNA by quantitative real-time PCR (FIG. 23G).

Expected Outcomes

This protocol contained a sequentially detailed steps for genetically manipulating NK-92 cells using Cas9 mediated gene knockout as well as dCas9-mediated transcriptional repression and activation of genes. Optimized and working sgRNA sequences was also provided which might be used by others to functionally validate the successful establishment of the Cas9⁺ and dCas9⁺ NK-92 cell lines. The effects of targeting specific genes in human NK cells on homeostasis, function, and therapeutic potential of NK cells might be studied using the current Cas9⁺ and dCas9⁺ NK-92 platforms. The current protocols might be anticipated to be extended to modify human NK cells derived from cord blood or peripheral blood of healthy donors using CRISPR for developing off-the-shelf NK cell-based therapies (Suck et al., 2016).

The data suggested that the five major steps of the protocol, namely, transduction of NK cells with Cas9/dCas9, sorting of Cas9/dCas9⁺ NK cells, single cell cloning of sorted Cas9/dCas9⁺ NK cells, confirmation of cytotoxic function, and sgRNA-based validation of the CRISPR-engineered NK cells were feasible. However, at each of these steps, it was likely that the desired outcome might not be obtained due to technical difficulties. Therefore, in the troubleshooting section, potential pitfalls were outlined and solutions to ensure that each of the steps were provided leading to generation and validation of CRISPR-engineered NK cells was successful.

Quantification and Statistical Analysis

Before proceeding with CRISPR-mediated target gene modification in Cas9⁺ and dCas9⁺ NK-92 cells, it was ensured that transduction with Cas9 or dCas9 did not impair NK cell viability, proliferation, and cytotoxicity. Only single cell Cas9⁺ and dCas9⁺ NK-92 clones without disruptions in normal NK homeostasis were selected for functional validation and would be used by us in future experiments. For manipulating a given gene in Cas9⁺ and dCas9⁺ NK-92 cells during functional validation of the NK cell platforms, two top-scoring sgRNAs was selected with low off-target effects using the Broad Institute Genetic Perturbation Platform (GPP) web portal (Doench et al., 2016). The abilities were tested of each of these sgRNAs to knockout (Cas9), repress transcription (dCas9-KRAB), and activate transcription (dCas9-VP64) of genes in NK-92 cells. It was found that for the Cas9 and dCas9-KRAB systems, both top scoring sgRNAs had identical effects. However, in the dCas9-VP64 system, only one sgRNA was successful in transcriptionally activating the target gene and was shown. For experiments aimed at modulating specific genes of interest in these platforms, testing at least 2 sgRNAs per gene was recommended, as demonstrated. Cytotoxicity assays and assays for functional validation of the Cas9 and dCas9 systems were conducted in three technical replicates per sample. P-values have been calculated using the Student's t-test in GraphPad software. The level of statistical significance for all experiments has been set at P<0.05.

Considerations

As innate immune cells, NK cells were generally refractory to viral transductions (Schmidt et al., 2021). For this reason, other groups have previously electroporated the Cas9 ribonucleoprotein complex into primary human NK cells (Pomeroy et al., 2020, Rautela et al., 2020, Berrien-Elliott et al., 2020). While electroporation was suitable for knocking out genes where transient expression of Cas9 protein was sufficient, dCas9-KRAB-mediated transcriptional repression and dCas9-VP64-mediated transcriptional activation require constitutive expression of dCas9-KRAB and dCas9-VP64, respectively. Therefore, NK-92 lymphoblasts, which were easier to manipulate using lentiviruses than normal NK cells, were used. Furthermore, because NK-92 cell-based therapies were currently in clinical trials for both hematological malignancies and solid tumors ((Li et al., 2015, Tang et al., 2018, Chu et al., 2014, Rezvani and Rouce, 2015, Klingemann et al., 2016), https://immunitybio.com/pipeline/), it was anticipated that the current protocols for CRISPR-based editing of NK-92 cells would have clinical applications.

Although the current protocol contains the detailed steps for engineering NK-92 cells using CRISPR, it might be extended to other malignant NK cell lines including NKL (Robertson et al., 1996), and NK101 (Yang et al., 2019) which were also being used in research and clinical applications. In the future, it was intended to genetically manipulate primary human NK cells derived from cord blood or from peripheral blood of healthy donors using the above-described CRISPR-based methods. For modifying healthy human NK cells, it was acknowledged that the current protocol might have to be modified because normal NK cells were more difficult to transduce than NK cell lines (Schmidt et al., 2021). At present, it was difficult to predict the extent of modification necessary to constitutively edit healthy NK cells for CRISPR-based applications. However, it was expected that the sequence of steps to constitutively modify healthy NK cells for CRISPR-based applications to be identical to that described here for NK-92.

Because it was used as fresh lentiviral supernatant for transducing NK-92 without determining the lentiviral titer, frequencies of transduced cells vary in each experiment. Unfortunately, determining lentivirus titer using permissive cell lines including HEK cells does not directly translate to innate immune cells such as NK cells. Also, MOI for estimating efficiencies of Cas9- and dCas9-transductions was not recommended in NK-92 cells for two reasons:

First, increasing the MOI does not significantly improve transduction efficiencies of human NK cells (Su et al., 2011, Rautela et al., 2020), suggesting that MOI was not a reliable measure of estimating NK transduction efficiencies.

Second, a more recent study suggested that transduction efficiencies of cells in culture could not be accurately estimated using MOI because cell cultures were heterogenous with some cells being more resistant than others to transduction and because virions might not contact each cell with the same efficiency. Furthermore, when a virus particle interacts with and infect the cell, it was a discrete event. Even at MOI=1, the same cell might become infected many times. Therefore, MOI might only be applied to a homogeneous cell population with each cell having identical transduction efficiency (Shabram and Aguilar-Cordova, 2000), which was not the case with NK cells cultures. In the current experiments, this situation was further complicated by the general resistance of all human NK cells to viral transduction (Schmidt et al., 2021).

Instead of using MOI, users of the current protocol was recommended to apply 1% of transduced cells (on day 2 post infection), as the minimum cut off in Cas9 and dCas9 transduction experiments to facilitate expansion and sorting of these cells for next steps in CRISPR engineering.

The constitutive expression of the active Cas9 nuclease in NK-92 cells in the CRISPRko system might lead to off-target and deleterious genomic alterations in the NK cells. There were several methods to mitigate this potential problem. One method was to use electroporation of the Cas9 ribonucleoprotein (RNP) which would lower the time frame for which active Cas9 was present in the NK cell, thereby reducing the risk of off-target genetic alterations (Hildreth et al., 2020).

A better solution which might avoid the use of Cas9 but achieved the same effect was the dCas9-KRAB system (Gilbert et al., 2013). In this system, as Cas9 was catalytically dead and the specificity of the gene targeted for transcriptional repression was decided by the sgRNA sequence (Qi et al., 2013), the off-target toxicity of Cas9 was drastically reduced. Furthermore, unlike the Cas9 system which knocked out the entire protein, the sgRNAs in the dCas9-KRAB system might be tailored to modulate the level of transcriptional repression of a target gene. Such downmodulation was especially important while engineering NK cells for therapeutic applications where complete knockout of a given gene using Cas9 might be detrimental to the NK cell.

Troubleshooting

No transduction or low frequencies of Cas9- and dCas9-transduced NK-92 cells

Potential solutions: Because NK cells were innate immune cells, they were difficult to transduce with lentiviruses (Schmidt et al., 2021). It was found that low levels of transduction (<1%) were mostly attributed to the lentivirus quality/titer. Potential solutions for this problem include:

A fresh lot of virus was made, and on day 4 of the virus production, filtering of the lentiviral supernatant was avoided but rather centrifuged (300×g for 5 min) it to remove debris before concentrating the virus. At the final step of virus concentration on day 5, using 500 plf fresh viral supernatant to transduce the NK-92 cells as freeze thawing of viral supernatant might affect the lentivirus quality and titer. Tracking the cells with low transduction efficiencies for at least a week. In some cases, although the efficiency of transduction was initially low, after a week the frequencies of GFP⁺ transduced cells were found to be increased (FIG. 22A).

Transduction with Cas9 or dCas9 slows down or blocks the proliferation of NK cells

Potential solutions: the following solutions was suggested to overcome the potential pitfall 2.

One must ensure that the NK-92 cell growth media was being supplemented with rhIL-2 (100 U/mL). Because NK-92 cell proliferation was affected by the seeding density at the time of culture initiation, the post-sort Cas9⁺ or dCas9⁺ NK-92 cells were suggested to be grown in 96-well plates at high densities (˜0.5×10⁶/well) and cells to 48-well plates were transferred and finally into T-25 flasks when these cells began proliferating. If this method doesn't work, one would infer that the reduced proliferation of transduced NK-92 cells might be caused by lentiviral integrations that disrupt genes involved in NK-92 cell proliferation. The only recourse in this case would be to restart the experiment. Depending on the type of application, the users were recommended to either proceed further after checking NK-92 proliferation or troubleshoot. For example, proliferation might be less critical for clinical applications where NK cytotoxicity was the most important and NK cells were irradiated before use (Klingemann et al., 2016, Tang et al., 2018).

Contamination of sorted and expanding Cas9⁺ and dCas9⁺ single cell clones with untransduced GFP⁻ cells

Potential solutions: FACS-based purification protocols did not led to the isolation of the desired populations at 100% purity. For example, it was observed that there was an outgrowth of DAPI⁻GFP⁻ cells after expanding some of the sorted dCas9-VP64⁺ single cell clones (FIG. 23E). Two potential solutions were proposed for this problem:

Polyclonal dCas9-VP64⁺ was sorted to remove DAPI⁻GFP⁻ cells and single cell cloning was performed. Multiple 96 well plates was seeded with single cell clones to get at least ˜5-10 pure single cell colonies of dCas9-VP64⁺ NK-92 cells.

None of the selected clones show cytotoxicity at levels comparable to that of unmodified NK-92 cells

Potential solutions: Seeding of multiple (˜5-10) single cells clones were suggested to increase the chances of getting at least one Cas9/dCas9⁺ NK cell clone with cytotoxicity comparable to that of untransduced NK-92 cells. It was observed that cytotoxicity varies at a clonal level, i.e., a Cas9/dCas9-transduced polyclonal NK population with cytotoxicity comparable to that of untransduced cells did not guarantee that single cells clones would be equally cytotoxic and vice-versa. For example, in FIG. 22E it was observed that polyclonal dCas9-KRAB NK-92 cells had lower cytotoxicity whereas clone-1 of same population showed cytotoxicity at levels similar to untransduced NK-92 cells.

Transduction with the sgRNA did not yield the desired effect, i.e., knockout or transcriptional activation or transcriptional repression.

Potential solutions: ˜2-5 sgRNAs per gene was selected using the Broad Institute Genetic Perturbation Platform (Doench et al., 2016) which increased the likelihood of finding the best working sgRNA, which further yielded the desired gene regulation.

Resource Availability

All plasmids described in this study have been deposited in Addgene. The NK-92, K-562 and Lenti-X 293T cell lines were obtained from American Type Culture Collection and Takara Bio respectively. The research described in this manuscript is covered by a pending US patent application.

INFORMAL SEQUENCE LISTING
Control single guide (sg) RNA,
forward sequence (CRISPRko/i/a):
GTAGCGAACGTGTCCGGCGT (SEQ ID NO: 1)
Control single guide (sg) RNA,
reverse sequence (CRISPRko/i/a):
ACGCCGGACACGTTCGCTAC (SEQ ID NO: 2)
RFP sgRNA, forward sequence (CRISPRko):
GTCACCACATACGAAGACGG (SEQ ID NO: 3)
RFP sgRNA, reverse sequence (CRISPRko):
CCGTCTTCGTATGTGGTGAC (SEQ ID NO: 4)
IL-2Rγ sgRNA-1, forward sequence (CRISPRko):
GGTGCAGTACCGGACTGACT (SEQ ID NO: 5)
IL-2Rγ sgRNA-1, reverse sequence (CRISPRko):
AGTCAGTCCGGTACTGCACC (SEQ ID NO: 6)
IL-2Rγ sgRNA-2, forward sequence (CRISPRko):
AGCCGCTTTAACCCACTCTG (SEQ ID NO: 7)
IL-2Rγ sgRNA-2, reverse sequence (CRISPRko):
(SEQ ID NO: 8)
CAGAGTGGGTTAAAGCGGCT
CRISPRa IL-15 sgRNA,
forward sequence (CRISPRa):
ACACCTCCCGCGGAGACTGG (SEQ ID NO: 9)
CRISPRa IL-15 sgRNA,
reverse sequence (CRISPRa):
CCAGTCTCCGCGGGAGGTGT (SEQ ID NO: 10)
Primer sequences in qPCR,
IL-15, forward sequence:
AACAGAAGCCAACTGGGTGAATG
(SEQ ID NO: 11)
Primer sequences in qPCR,
IL-15, reverse sequence:
CTCCAAGAGAAAGCACTTCATTGC (SEQ ID NO: 12)
Primer sequences in qPCR,
GAPDH, forward sequence:
GTCTCCTCTGACTTCAACAGCG (SEQ ID NO: 13)
Primer sequences in qPCR,
GAPDH, reverse sequence:
ACCACCCTGTTGCTGTAGCCAA (SEQ ID NO: 14)
IL2RG sgRNA (1)-Forward Sequence (CRISPRi)
TGGTAATGATGGCTTCAACA (SEQ ID NO: 15)
IL2RG sgRNA (1)-Reverse Sequence (CRISPRi)
TGTTGAAGCCATCATTACCA (SEQ ID NO: 16)
L2RG sgRNA (2)-Forward Sequence (CRISPRi)
AGGGATGTGAATGGTAATGA (SEQ ID NO: 17)
L2RG sgRNA (2)-Reverse Sequence (CRISPRi)
TCATTACCATTCACATCCCT (SEQ ID NO: 18)
hU6 primer
GAGGGCCTATTTCCCATGAT (SEQ ID NO: 19)
dCas9 (SEQ ID NO: 20)
MDKKYSIGLAIGTNSVGWAVITDEYKVPSKKFKVLGNTDR
HSIKKNLIGALLFDSGETAEATRLKRTARRRYTRRKNRIC
YLQEIFSNEMAKVDDSFFHRLEESFLVEEDKKHERHPIFG
NIVDEVAYHEKYPTIYHLRKKLVDSTDKADLRLIYLALAH
MIKFRGHFLIEGDLNPDNSDVDKLFIQLVQTYNQLFEENP
INASGVDAKAILSARLSKSRRLENLIAQLPGEKKNGLFGN
LIALSLGLTPNFKSNFDLAEDAKLQLSKDTYDDDLDNLLA
QIGDQYADLFLAAKNLSDAILLSDILRVNTEITKAPLSAS
MIKRYDEHHQDLTLLKALVRQQLPEKYKEIFFDQSKNGYA
GYIDGGASQEEFYKFIKPILEKMDGTEELLVKLNREDLLR
KQRTFDNGSIPHQIHLGELHAILRRQEDFYPFLKDNREKI
EKILTFRIPYYVGPLARGNSRFAWMTRKSEETITPWNFEE
VVDKGASAQSFIERMTNFDKNLPNEKVLPKHSLLYEYFTV
YNELTKVKYVTEGMRKPAFLSGEQKKAIVDLLFKTNRKVT
VKQLKEDYFKKIECFDSVEISGVEDRFNASLGTYHDLLKI
IKDKDFLDNEENEDILEDIVLTLTLFEDREMIEERLKTYA
HLFDDKVMKQLKRRRYTGWGRLSRKLINGIRDKQSGKTIL
DFLKSDGFANRNFMQLIHDDSLTFKEDIQKAQVSGQGDSL
HEHIANLAGSPAIKKGILQTVKVVDELVKVMGRHKPENIV
IEMARENQTTQKGQKNSRERMKRIEEGIKELGSQILKEHP
VENTQLQNEKLYLYYLQNGRDMYVDQELDINRLSDYDVDA
IVPQSFLKDDSIDNKVLTRSDKNRGKSDNVPSEEVVKKMK
NYWRQLLNAKLITQRKFDNLTKAERGGLSELDKAGFIKRQ
LVETRQITKHVAQILDSRMNTKYDENDKLIREVKVITLKS
KLVSDFRKDFQFYKVREINNYHHAHDAYLNAVVGTALIKK
YPKLESEFVYGDYKVYDVRKMIAKSEQEIGKATAKYFFYS
NIMNFFKTEITLANGEIRKRPLIETNGETGEIVWDKGRDF
ATVRKVLSMPQVNIVKKTEVQTGGFSKESILPKRNSDKLI
ARKKDWDPKKYGGFDSPTVAYSVLVVAKVEKGKSKKLKSV
KELLGITIMERSSFEKNPIDFLEAKGYKEVKKDLIIKLPK
YSLFELENGRKRMLASAGELQKGNELALPSKYVNFLYLAS
HYEKLKGSPEDNEQKQLFVEQHKHYLDEIIEQISEFSKRV
ILADANLDKVLSAYNKHRDKPIREQAENIIHLFTLTNLGA
PAAFKYFDTTIDRKRYTSTKEVLDATLIHQSITGLYETRI
DLSQLGGD
KRAB (SEQ ID NO: 21)
DAKSLTAWSRTLVTFKDVFVDFTREEWKLLDTAQQIVYRN
VMLENYKNLVSLGYQLTKPDVILRLEKGEEP
Tegument protein VP16 (P06492)
(SEQ ID NO: 22)
MDLLVDELFADMNADGASPPPPRPAGGPKNTPAAPPLYAT
GRLSQAQLMPSPPMPVPPAALENRLLDDLGFSAGPALCTM
LDTWNEDLFSALPTNADLYRECKFLSTLPSDVVEWGDAYV
PERTQIDIRAHGDVAFPTLPATRDGLGLYYEALSRFFHAE
LRAREESYRTVLANFCSALYRYLRASVRQLHRQAHMRGRD
RDLGEMLRATIADRYYRETARLARVLFLHLYLFLTREILW
AAYAEQMMRPDLFDCLCCDLESWRQLAGLFQPFMFVNGAL
TVRGVPIEARRLRELNHIREHLNLPLVRSAATEEPGAPLT
TPPTLHGNQARASGYFMVLIRAKLDSYSSFTTSPSEAVMR
EHAYSRARTKNNYGSTIEGLLDLPDDDAPEEAGLAAPRLS
FLPAGHTRRLSTAPPTDVSLGDELHLDGEDVAMAHADALD
DFDLDMLGDGDSPGPGFTPHDSAPYGALDMADFEFEQMFT
DALGIDEYGG
Transcription factor p65 (Q04206)
(SEQ ID NO: 23):
MDELFPLIFPAEPAQASGPYVEIIEQPKORGMRFRYKCEG
RSAGSIPGERSTDTTKTHPTIKINGYTGPGTVRISLVTKD
PPHRPHPHELVGKDCRDGFYEAELCPDRCIHSFONLGIQC
VKKRDLEQAISQRIQTNNNPFQVPIEEQRGDYDLNAVRLC
FQVTVRDPSGRPLRLPPVLSHPIFDNRAPNTAELKICRVN
RNSGSCLGGDEIFLLCDKVQKEDIEVYFTGPGWEARGSFS
QADVHRQVAIVFRTPPYADPSLQAPVRVSMOLRRPSDREL
SEPMEFQYLPDTDDRHRIEEKRKRTYETFKSIMKKSPFSG
PTDPRPPPRRIAVPSRSSASVPKPAPQPYPFTSSLSTINY
DEFPTMVFPSGQISQASALAPAPPQVLPQAPAPAPAPAMV
SALAQAPAPVPVLAPGPPQAVAPPAPKPTQAGEGTLSEAL
LQLQFDDEDLGALLGNSTDPAVFTDLASVDNSEFQQLLNQ
GIPVAPHTTEPMLMEYPEAITRLVTGAQRPPDPAPAPLGA
PGLPNGLLSGDEDFSSIADMDFSALLSQISS
Rta (SEQ ID NO: 24):
MRPKKDGLEDFLRLTPEIKKOLGSLVSDYCNVLNKEFTAG
SVEITLRSYKICKAFINEAKAHGREWGGLMATLNICNFWA
ILRNNRVRRRAENAGNDACSIACPIVMRYVLDHLIVVTDR
FFIQAPSNRVMIPATIGTAMYKLLKHSRVRAYTYSKVLGV
DRAAIMASGKQVVEHLNRMEKEGLLSSKFKAFCKWVFTYP
VLEEMFQTMVSSKTGHLTDDVKDVRALIKTLPRASYSSHA
GQRSYVSGVLPACLLSTKSKAVETPILVSGADRMDEELMG
NDGGASHTEARYSESGQFHAFTDELESLPSPTMPLKPGAQ
SADCGDSSSSSSDSGNSDTEQSEREEARAEAPRLRAPKSR
RTSRPNRGQTPCPSNAAEPEQPWIAAVHQESDERPIFPHP
SKPTFLPPVKRKKGLRDSREGMFLPKPEAGSAISDVFEGR
EVCQPKRIRPFHPPGSPWANRPLPASLAPTPTGPVHEPVG
SLTPAPVPQPLDPAPAVTPEASHLLEDPDEETSQAVKALR
EMADTVIPQKEEAAICGQMDLSHPPPRGHLDELTTTLESM
TEDLNLDSPLTPELNEILDTELNDECLLHAMHISTGLSIF
DTSLF
VP64 (SEQ ID NO: 25):
DALDDFDLDMLGSDALDDFDLDMLGSDALDDFDLDMLGSD
ALDDFDLDML
Transactivation domain of p65 (SEQ ID NO: 26):
TQAGEGTLSEALLQLQFDDEDLGALLGNSTDPAVFTDLAS
VDNSEFQQLLNOGIPVAPHTTEPMLMEYPEAITRLVTGAQ
RPPDPAPAPLGAPGLPNGLLSGDEDESSIADMDESALLSQ
IS

1. A Natural Killer (NK) cell comprising an integrated nucleic acid sequence encoding a catalytically inactive Cas9 (dCas9) protein.

2. The NK cell of claim 1, wherein said NK cell is obtained by transduction of an exogenous nucleic acid encoding said catalytically inactive Cas9 (dCas9) protein.

3. The NK cell of claim 2, wherein the exogenous nucleic acid encoding said catalytically inactive Cas9 (dCas9) protein is present within a lentiviral vector.

4. (canceled)

5. The NK cell of claim 1, wherein said NK cell further comprises a nucleic acid encoding an sgRNA sequence.

6. (canceled)

7. The NK cell of claim 1, wherein the NK cell constitutively expresses said dCas9 protein.

8. A substantially pure population of Natural Killer (NK) cells, wherein each of said NK cells comprises an integrated nucleic acid sequence encoding a Cas9 protein.

9.-11. (canceled)

12. The substantially pure population of NK cells of claim 8, wherein said NK cells further comprise a nucleic acid encoding an sgRNA sequence.

13. (canceled)

14. The substantially pure population of NK cells of claim 8, wherein the NK cells constitutively express said Cas9 protein.

15. A substantially pure population of Natural Killer (NK) cells, wherein each of said NK cells comprises an integrated nucleic acid sequence encoding a catalytically inactive Cas9 (dCas9) protein.

16.-20. (canceled)

21. The substantially pure population of NK cells of claim 15, wherein the dCas9 protein is constitutively expressed.

22. A method of preparing a plurality of Natural Killer (NK) cells each comprising an integrated nucleic acid sequence encoding a Cas9 protein, the method comprising:
(a) contacting a plurality of NK cells with a vector comprising a nucleic acid encoding a Cas9 protein thereby producing a transduced NK Cell wherein said nucleic acid encoding said Cas9 protein is integrated into the genome of said NK Cell;
(b) separating said transduced NK cell from said plurality of NK cells; and
(c) expanding said transduced NK cell thereby producing a plurality of natural Killer (NK) cells each comprising an integrated nucleic acid sequence encoding said Cas9 protein.

23.-28. (canceled)

29. A plurality of Natural Killer (NK) cells each comprising an integrated nucleic acid sequence encoding a Cas9 protein obtained by the method of claim 22.

30. A method of preparing a plurality of Natural Killer (NK) cells each comprising an integrated nucleic acid sequence encoding a catalytically inactive Cas9 (dCas9) protein, the method comprising:
(a) contacting a plurality of NK cells with a vector comprising a nucleic acid encoding a dCas9 protein thereby producing a transduced NK Cell wherein said nucleic acid encoding said dCas9 protein is integrated into the genome of said NK Cell;
(b) separating said transduced NK cell from said plurality of NK cells; and
(c) expanding said transduced NK cell thereby producing a plurality of natural Killer (NK) cells each comprising an integrated nucleic acid sequence encoding said dCas9 protein.

31.-35. (canceled)

36. The method of claim 30, wherein the dCas9 protein is fused to a transcriptional activator or a transcriptional repressor.

37.-41. (canceled)

42. A plurality of Natural Killer (NK) cells each comprising an integrated nucleic acid sequence encoding a dCas9 protein obtained by the method of claim 30.

43. A method of increasing expression of a target gene within a subject, the method comprising administering to the subject a plurality of Natural Killer (NK) cells, wherein each of said plurality NK cell comprises an integrated nucleic acid sequence encoding a catalytically inactive Cas9 (dCas9).

44.-49. (canceled)

50. A method of decreasing the expression of a target gene within a subject, the method comprising administering to the subject a plurality of NK cells, wherein each of said plurality NK cells comprises an integrated nucleic acid sequence encoding a Cas9 protein.

51.-53. (canceled)

54. A method of treating or preventing a disease in a subject in need thereof, the method comprising administering to said subject a therapeutically effective amount of an NK cell capable of expressing an elevated level of a type I interferon or an elevated level of IL-15 relative to a standard control NK cell.

55.-72. (canceled)

73. A method of preventing recurrence of leukemia in a subject, the method comprising administering to said subject an effective amount of a natural killer (NK) cell capable of expressing an elevated level of a type I interferon or an elevated level of IL-15 relative to a standard control NK cell.

74. (canceled)

75. A method of treating or preventing an infectious disease in a subject having leukemia, the method comprising administering an effective amount of a natural killer (NK) cell capable of expressing an elevated level of a type I interferon or an elevated level of IL-15 relative to a standard control NK cell.

76.-78. (canceled)


SEQUENCE LISTING (26 sequences)

SEQ ID NO: 1 - DNA, 20 bp, Artificial Sequence
  Synthetic polynucleotide
gtagcgaacgtgtccggcgt

SEQ ID NO: 2 - DNA, 20 bp, Artificial Sequence
  Synthetic polynucleotide
acgccggacacgttcgctac

SEQ ID NO: 3 - DNA, 20 bp, Artificial Sequence
  Synthetic polynucleotide
gtcaccacatacgaagacgg

SEQ ID NO: 4 - DNA, 20 bp, Artificial Sequence
  Synthetic polynucleotide
ccgtcttcgtatgtggtgac

SEQ ID NO: 5 - DNA, 20 bp, Artificial Sequence
  Synthetic polynucleotide
ggtgcagtaccggactgact

SEQ ID NO: 6 - DNA, 20 bp, Artificial Sequence
  Synthetic polynucleotide
agtcagtccggtactgcacc

SEQ ID NO: 7 - DNA, 20 bp, Artificial Sequence
  Synthetic polynucleotide
agccgctttaacccactctg

SEQ ID NO: 8 - DNA, 20 bp, Artificial Sequence
  Synthetic polynucleotide
cagagtgggttaaagcggct

SEQ ID NO: 9 - DNA, 20 bp, Artificial Sequence
  Synthetic polynucleotide
acacctcccgcggagactgg

SEQ ID NO: 10 - DNA, 20 bp, Artificial Sequence
  Synthetic polynucleotide
ccagtctccgcgggaggtgt

SEQ ID NO: 11 - DNA, 23 bp, Artificial Sequence
  Synthetic polynucleotide
aacagaagccaactgggtgaatg

SEQ ID NO: 12 - DNA, 24 bp, Artificial Sequence
  Synthetic polynucleotide
ctccaagagaaagcacttcattgc

SEQ ID NO: 13 - DNA, 22 bp, Artificial Sequence
  Synthetic polynucleotide
gtctcctctgacttcaacagcg

SEQ ID NO: 14 - DNA, 22 bp, Artificial Sequence
  Synthetic polynucleotide
accaccctgttgctgtagccaa

SEQ ID NO: 15 - DNA, 20 bp, Artificial Sequence
  Synthetic polynucleotide
tggtaatgatggcttcaaca

SEQ ID NO: 16 - DNA, 20 bp, Artificial Sequence
  Synthetic polynucleotide
tgttgaagccatcattacca

SEQ ID NO: 17 - DNA, 20 bp, Artificial Sequence
  Synthetic polynucleotide
agggatgtgaatggtaatga

SEQ ID NO: 18 - DNA, 20 bp, Artificial Sequence
  Synthetic polynucleotide
tcattaccattcacatccct

SEQ ID NO: 19 - DNA, 20 bp, Artificial Sequence
  Synthetic polynucleotide
gagggcctatttcccatgat

SEQ ID NO: 20 - protein, 1368 aa, Artificial Sequence
  Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 71 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEP

SEQ ID NO: 22 - protein, 490 aa, Homo sapiens
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 23 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 24 - protein, 605 aa, Human gammaherpesvirus 4
MRPKKDGLEDFLRLTPEIKKQLGSLVSDYCNVLNKEFTAGSVEITLRSYKICKAFINEAKAHGREWGGLMATLNICNFWAILRNNRVRRRAENAGNDACSIACPIVMRYVLDHLIVVTDRFFIQAPSNRVMIPATIGTAMYKLLKHSRVRAYTYSKVLGVDRAAIMASGKQVVEHLNRMEKEGLLSSKFKAFCKWVFTYPVLEEMFQTMVSSKTGHLTDDVKDVRALIKTLPRASYSSHAGQRSYVSGVLPACLLSTKSKAVETPILVSGADRMDEELMGNDGGASHTEARYSESGQFHAFTDELESLPSPTMPLKPGAQSADCGDSSSSSSDSGNSDTEQSEREEARAEAPRLRAPKSRRTSRPNRGQTPCPSNAAEPEQPWIAAVHQESDERPIFPHPSKPTFLPPVKRKKGLRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 25 - protein, 50 aa, Artificial Sequence
  Synthetic polypeptide
DALDDFDLDMLGSDALDDFDLDMLGSDALDDFDLDMLGSDALDDFDLDML

SEQ ID NO: 26 - protein, 123 aa, Homo sapiens
TQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS